(19) United States
(12) Patent Application Publication
(10) Pub. No.: US 2014/0329797 A1
(43) Pub. Date: Nov. 6, 2014

(54) SUBSTITUTED OXADIAZOLYL PYRIDINONES AND OXADIAZOLYL PYRIDAZINONES AS HIF INHIBITORS

(71) Applicant: Bayer Intellectual Property GmbH, Monheim (DE)

(72) Inventors: Michael Härter, Leverkusen (DE); Hartmut Beck, Wuppertal (DE); Peter Ellinghaus, Melle (DE); Kerstin Unterschemmann, Essen (DE)

(73) Assignee: BAYER INTELLECTUAL PROPERTY GMBH, Monheim (DE)

(21) Appl. No.: 14/352,379

(22) PCT Filed: Oct. 16, 2012
(86) PCT No.: PCT/EP2012/070484

§ 371 Date: Jul. 21, 2014

(30) Foreign Application Priority Data
Oct. 17, 2011 (EP) 11185480.8

(51) Int. Cl. C07D 413/14; A61K 31/501; C07D 413/04; A61K 31/4545; A61K 31/4439; A61K 31/5377; A61K 31/496

(52) U.S. Cl. CPC C07D 413/14; A61K 31/5377; A61K 31/501; A61K 31/496; A61K 31/4545; A61K 31/4439; C07D 413/04
USPC 514/210.18; 514/210.2; 514/236.2; 514/252.05; 514/253.1; 514/318; 514/340; 514/343; 544/131; 544/238; 544/364; 546/194; 546/269.4

(57) ABSTRACT

The present application relates to novel substituted 5-(1,2,4-oxadiazol-5-yl)pyridin-2-ones and 6-(1,2,4-oxadiazol-5-yl)pyridazin-3-ones, to processes for their preparation, to their use for the treatment and/or prevention of diseases and to their use for producing medicaments for treatment and/or prevention of diseases, in particular for treatment and/or prevention of hyperproliferative and angiogenic diseases and those diseases which arise from metabolic adaptation to hypoxic states. Such treatments can be effected in the form of monotherapy or else in combination with other medicaments or further therapeutic measures.

The present application relates to novel substituted 5-(1,2,4-oxadiazol-5-yl)pyridin-2-ones and 6-(1,2,4-oxadiazol-5-yl)pyridazin-3-ones, to processes for their preparation, to their use for the treatment and/or prevention of diseases and to their use for producing medicaments for treatment and/or prevention of diseases, in particular for treatment and/or prevention of hyperproliferative and angiogenic diseases and those diseases which arise from metabolic adaptation to hypoxic states. Such treatments can be effected in the form of monotherapy or else in combination with other medicaments or further therapeutic measures.

Cancers are the consequence of uncontrolled cell growth of a wide variety of different tissues. In many cases the new cells penetrate into existing tissue (invasive growth), or they metastasize into remote organs. Cancers occur in a wide variety of different organs and often progress in a manner specific to the tissue. The term “cancer” as a generic term therefore describes a large group of defined diseases of different organs, tissue and cell types.

In 2002, 4.4 million people worldwide were diagnosed with tumours of the breast, intestine, ovaries, lung or prostate. In the same year, approx. 2.5 million deaths were assumed to be a consequence of these diseases (Globocan 2002 Report). In the USA alone, in 2005, more than 1.25 million new cases and more than 500000 deaths were predicted from cancers. The majority of these new cases relate to cancers of the intestine (˜100000), lung (˜170000), breast (˜210000) and prostate (˜230000). A further increase in cancers of approx. 15% over the next 10 years is expected (American Cancer Society, Cancer Facts and Figures. 2005).

Some tumours at early stages can be removed by surgical and radiotherapy measures. Metastasized tumours can generally only be treated palliatively by chemotherapeutics. The aim here is to achieve the optimum combination of an improvement in the quality of life and prolonging of life.

Chemotherapies are often composed of combinations of cytotoxic medicaments. The majority of these substances have bonding to tubulin as their mechanism of action, or they are compounds which interact with the formation and processing of nucleic acids. As of recently, these also include enzyme inhibitors which interfere with epigenetic DNA modification or cell cycle progression (e.g. histone deacetylase inhibitors, aurora kinase inhibitors). Since such therapies are toxic, there has recently been an increasing focus on targeted therapies in which specific processes in the cell are blocked without a high level of toxic stress. These especially include inhibitors of kinases which inhibit the phosphorylation of receptors and signal transmission molecules. One example thereof is imatinib, which is used very successfully for treatment of chronic myeloid leukaemia (CML) and gastrointestinal stromal tumours (GIST). Further examples are substances which block EGFR kinase and HER2, such as erlotinib, and VEGFR kinase inhibitors, such as sorafenib and sunitinib, which are used for kidney cell carcinomas, liver carcinomas and advanced stages of GIST.

With an antibody directed against VEGF, it has been possible to prolong the life expectancy of colorectal carcinoma patients. Bevacizumab inhibits the growth of blood vessels, which is an obstacle to rapid expansion of a tumour, since it requires connection to the blood vessel system for continuously functioning supply and disposal.

One stimulus for angiogenesis is hypoxia, which occurs time and again with solid tumours, since blood supply is inadequate because of the unregulated growth. If there is a lack of oxygen, cells switch their metabolism from oxidative phosphorylation to glycolysis, so as to stabilize the ATP level in the cell. This process is controlled by a transcription factor which is upregulated depending on the oxygen content in the cell. This transcription factor, called “hypoxia-induced factor” (HIF), is normally removed post-translationally by rapid degradation and prevented from being transported into the cell nucleus. This is accomplished by the hydroxylation of two proline units in the oxygen-degradable domain (ODD) and one asparagine unit in the vicinity of the C terminus by the enzymes prolyl dehydrogenase and FIH (“factor inhibiting HIF”). After the modification of the proline units, HIF can be degraded with mediation by the Hippel-Lindau protein (part of a ubiquitin-E3-ligase complex) via the proteasome apparatus (Maxwell, Wiesener et al., 1999). In the event of oxygen deficiency, the degradation does not take place and the protein is upregulated and leads to transcription or to blockage of the transcription of numerous (more than 100) other proteins (Semenza and Wang, 1992; Wang and Semenza, 1995).

The transcription factor HIF is formed by the regulated α-subunit and a constitutively present β-subunit (ARNT, aryl hydrocarbon receptor nuclear translocator). There are three different species of the α-subunit, 1α,2α and 3α, the latter being assumed to be a suppressor if anything (Makino, Cao et al., 2001). The HIF subunits are bHLH (basic helix loop helix) proteins which dimerize via their HLH and PAS (Per-Arnt-Sim) domains, which starts their transactivation activity (Jiang, Rue et al., 1996).

In the most important tumour entities, overexpression of the HIF1α protein is correlated with increasing blood vessel density and enhanced VEGF expression (Hirota and Semenza, 2006). At the same time, glucose metabolism is moved towards glycolysis, and the Krebs cycle is reduced in favour of the production of cell units. This also implies a change in lipid metabolism. Such changes appear to guarantee the survival of the tumours. If, on the other hand, the activity of HIF is now inhibited, the development of tumours could consequently be suppressed. This has already been observed in various experimental models (Chen, Zhao et al., 2003; Stoeltzing, McCarty et al., 2004; Li, Lin et al., 2005; Mizukami, Jo et al., 2005; Li, Shi et al., 2006). Specific inhibitors of the HIF-controlled metabolism should therefore be suitable as tumour therapeutics.

The object of the present invention was therefore to provide novel compounds which act as potent inhibitors of the transactivating action of the transcription factor HIF and can be employed as such for treatment and/or prevention of disorders, in particular of hyperproliferative and angiogenic disorders, such as cancer and tumour disorders.

WO 2005/030121-A2 and WO 2007/065010-A2 describe the suitability of certain pyrazole derivatives for inhibition of the expression of HIF and HIF-regulated genes in tumour cells, and WO 2008/141731-A2, WO 2010/054762-A1, WO 2010/054763-A1 and WO 2010/054764-A1 disclose heteroaryl-substituted pyrazole derivatives as inhibitors of the HIF regulatory path for the treatment of cancer disorders.

WO 03/068230-A1 and WO 2005/018557-A2 describe substituted pyridinones for the treatment of disorders which are mediated by p38 MAP kinase and/or tumour necrosis factor (TNF) activities.

Individual phenyl-substituted 5-(1,2,4-oxadiazol-5-yl)pyridin-2-one and 6-(1,2,4-oxadiazol-5-yl)pyridazin-3-one derivatives have been indexed by Chemical Abstracts as “Chemical Library” substances without literature reference [see 1-(4-chlorobenzyl)-5-[3-(4-methylphenyl)-1,2,4-oxadiazol-5-yl]pyridin-2(1H)-one, CAS Registry No. 1251606-94-6; 5-[3-(4-chlorophenyl)-1,2,4-oxadiazol-5-yl]-1-(4-methylbenzyl)pyridin-2(1H)-one, CAS Registry No. 1251607-33-6; 5-[3-(4-methoxyphenyl)-1,2,4-oxadiazol-5-yl]-1-(4-methylbenzyl)pyridin-2(1H)-one, CAS Registry No. 1251583-14-8; 2-(4-methoxybenzyl)-6-[3-(4-methylphenyl)-1,2,4-oxadiazol-5-yl]pyridazin-3(2H)-one, CAS Registry No. 1251635-10-5]. No therapeutic use for these compounds has been described to date.

The present invention provides compounds of the general formula (I)

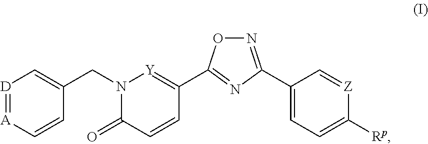

in which
either (i)
   A represents C—R^{A}
   and
   D represents CH or N
or (ii)
   A represents CH or N
and
   D represents C—R^{D},
where R^{A} represents chlorine, cyano, nitro, amino, (C₁-C₄)-alkyl, (C₁-C₄)-alkoxy or mono-(C₁-C₄)-alkylamino,
   where (C₁-C₄)-alkyl, (C₁-C₄)-alkoxy and mono-(C₁-C₄)-alkylamino may be substituted by hydroxy or up to three times by fluorine,
   and
   R^{D} represents (C₁-C₆)-alkyl, (C₃-C₆)-cycloalkyl, (C₁-C₄)-alkoxycarbonyl or a group of the formula —NR¹R² or —C(═O)—NR³R⁴,
      where (C₁-C₆)-alkyl is substituted by hydroxy or (C₁-C₄)-alkylcarbonyloxy and may additionally be substituted up to three times by fluorine
      and
      (C₃-C₆)-cycloalkyl is substituted by hydroxy, hydroxy-(C₁-C₄)-alkyl or (C₁-C₄)-alkylcarbonyloxy,
      and where
      R¹ and R² are attached to one another and together with the nitrogen atom to which they are attached form a 4- to 6-membered heterocycle which may contain a further heteroatom from the group consisting of N(R⁵), O, S and S(O)₂ and which may be substituted up to two times by identical or different radicals selected from the group consisting of fluorine, cyano, (C₁-C₄)-alkyl, hydroxy, methoxy and ethoxy,
         where (C₁-C₄)-alkyl for its part may be substituted by hydroxy or up to three times by fluorine
         and where
         R⁵ represents (C₁-C₄)-alkyl which may be substituted up to three times by fluorine or represents (C₃-C₆)-cycloalkyl, (C₁-C₄)-alkylcarbonyl or (C₁-C₄)-alkoxycarbonyl,
      and
      R³ and R⁴ independently of one another represent hydrogen or (C₁-C₄)-alkyl which may be substituted by hydroxy, methoxy, ethoxy or phenyl or up to three times by fluorine
      or
      R³ and R⁴ are attached to one another and have the meanings of R¹ and R²,
Y represents CH or N,
Z represents C—R^{m} or N, where
   R^{m} represents hydrogen, fluorine, chlorine, methyl or trifluoromethyl,
and
R^{p} represents halogen, cyano, pentafluorothio, tri-(C₁-C₄)-alkylsilyl, (C₁-C₆)-alkyl, (C₁-C₆)-alkoxy, (C₁-C₆)-alkylthio, (C₁-C₆)-alkylsulphonyl, (C₃-C₆)-cycloalkyl or 4- to 6-membered heterocyclyl,
   where (C₁-C₆)-alkyl, (C₁-C₆)-alkoxy, (C₁-C₆)-alkylthio and (C₁-C₆)-alkylsulphonyl for their part may be substituted by a radical selected from the group consisting of hydroxy, methoxy and ethoxy and up to six times by fluorine
   and
   (C₃-C₆)-cycloalkyl and 4- to 6-membered heterocyclyl for their part may be substituted up to two times by identical or different radicals selected from the group consisting of fluorine, (C₁-C₄)-alkyl, trifluoromethyl, hydroxy, methoxy and ethoxy,
and their salts, solvates and solvates of the salts,
except for the compounds
1-(4-chlorobenzyl)-5-[3-(4-methylphenyl)-1,2,4-oxadiazol-5-yl]pyridin-2(1H)-one,
5-[3-(4-chlorophenyl)-1,2,4-oxadiazol-5-yl]-1-(4-methylbenzyl)pyridin-2(1H)-one,
5-[3-(4-methoxyphenyl)-1,2,4-oxadiazol-5-yl]-1-(4-methylbenzyl)pyridin-2(1H)-one and
2-(4-methoxybenzyl)-6-[3-(4-methylphenyl)-1,2,4-oxadiazol-5-yl]pyridazin-3(2H)-one.

The invention furthermore provides the use of a compound from the group consisting of
1-(4-chlorobenzyl)-5-[3-(4-methylphenyl)-1,2,4-oxadiazol-5-yl]pyridin-2(1H)-one,
5-[3-(4-chlorophenyl)-1,2,4-oxadiazol-5-yl]-1-(4-methylbenzyl)pyridin-2(1H)-one,
5-[3-(4-methoxyphenyl)-1,2,4-oxadiazol-5-yl]-1-(4-methylbenzyl)pyridin-2(1H)-one
   and
2-(4-methoxybenzyl)-6-[3-(4-methylphenyl)-1,2,4-oxadiazol-5-yl]pyridazin-3(2H)-one
   for the treatment and/or prevention of diseases, in particular cancer and tumour disorders.

Compounds according to the invention or compounds which can be used according to the invention, hereinbelow together also referred to as compounds according to the invention, are the compounds of the formula (I) and their salts, solvates and solvates of the salts, the compounds, comprised by formula (I), of the formulae mentioned below and their salts, solvates and solvates of the salts and the compounds comprised by formula (I), mentioned below as working examples, and their salts, solvates and solvates of the salts, if the compounds, comprised by formula (I), mentioned below are not already salts, solvates and solvates of the salts.

The compounds according to the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers and the respective mixtures thereof. The stereoisomerically homogeneous constituents can be isolated from such mixtures of enantiomers and/or diastereomers in a known manner; chromatography processes are preferably used for this purpose, especially HPLC chromatography on an achiral or chiral phase.

Where the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²F, ³³F, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example to an extension of the half-life in the body or to a reduction in the active dose required; such modifications of the compounds according to the invention may therefore in some cases also constitute a preferred embodiment of the present invention. Isotopic variants of the compounds according to the invention can be prepared by generally customary processes known to those skilled in the art, for example by the methods described below and the procedures reported in the working examples, by using corresponding isotopic modifications of the particular reagents and/or starting compounds therein.

In the context of the present invention, preferred salts are physiologically acceptable salts of the compounds according to the invention. Also encompassed are salts which are not themselves suitable for pharmaceutical applications but can be used, for example, for isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, N,N-ethyldiisopropylamine, monoethanolamine, diethanolamine, triethanolamine, dimethylaminoethanol, diethylaminoethanol, procaine, dicyclohexylamine, dibenzylamine, N-methylpiperidine, N-methylmorpholine, arginine, lysine and 1,2-ethylenediamine.

In the context of the invention, solvates refer to those forms of the compounds according to the invention which, in the solid or liquid state, form a complex by coordination with solvent molecules. Hydrates are a specific form of solvates in which the coordination is with water. Preferred solvates in the context of the present invention are hydrates.

The N-oxides of pyridyl rings and tertiary cyclic amine groupings contained in compounds according to the invention are likewise embraced by the present invention.

Moreover, the present invention also encompasses prodrugs of the compounds according to the invention. Here, the term “prodrugs” refers to compounds which for their part can be biologically active or inactive, but are converted (for example metabolically or hydrolytically) into compounds according to the invention during their dwell time in the body.

In the context of the present invention, the substituents, unless specified otherwise, are each defined as follows:

In the context of the invention, (C₁-C₆)-alkyl, (C₂-C₆)-alkyl, (C₁-C₄)-alkyl and (C₂-C₄)-alkyl represent a straight-chain or branched alkyl radical having 1 to 6, 2 to 6, 1 to 4 and 2 to 4 carbon atoms, respectively. The following may be mentioned by way of example and by way of preference: methyl, ethyl, n-propyl, isopropyl, n-butyl, isobutyl, sec-butyl, tert-butyl, n-pentyl, 2-pentyl, 3-pentyl, neopentyl, n-hexyl, 2-hexyl and 3-hexyl.

In the context of the invention, hydroxy-C₁-C₄-alkyl represents a straight-chain or branched alkyl radical having 1 to 4 carbon atoms which carries a hydroxyl group as a substituent in the chain or in a terminal position. The following may be mentioned by way of example and by way of preference: hydroxymethyl, 1-hydroxyethyl, 2-hydroxyethyl, 1-hydroxy-1-methylethyl, 1,1-dimethyl-2-hydroxyethyl, 1-hydroxypropyl, 2-hydroxypropyl, 3-hydroxypropyl, 1-hydroxy-2-methylpropyl, 2-hydroxy-1-methylpropyl, 2-hydroxy-2-methylpropyl, 1-hydroxybutyl, 2-hydroxybutyl, 3-hydroxybutyl and 4-hydroxybutyl.

In the context of the invention, tri-(C₁-C₄)-alkylsilyl represents a silyl group having three identical or different straight-chain or branched alkyl substituents each having 1 to 4 carbon atoms. The following may be mentioned by way of example and by way of preference: trimethylsilyl, tert-butyldimethylsilyl and triisopropylsilyl.

In the context of the invention, mono-(C₁-C₄)-alkylamino represents an amino group having a straight-chain or branched alkyl substituent having 1 to 4 carbon atoms. The following may be mentioned by way of example and by way of preference: methylamino, ethylamino, n-propylamino, isopropylamino, n-butylamino, isobutylamino, sec-butylamino and tert-butylamino.

In the context of the invention, (C₁-C₆)-alkylthio, (C₂-C₆)-alkylthio and (C₂-C₄)-alkylthio represent a straight-chain or branched alkylthio radical (also referred to as alkylsulphanyl radical) having 1 to 6, 2 to 6 and 2 to 4 carbon atoms, respectively. The following may be mentioned by way of example and by way of preference: methylthio, ethylthio, n-propylthio, isopropylthio, n-butylthio, isobutylthio, sec-butylthio, tert-butylthio, n-pentylthio, 2-pentylthio, 3-pentylthio, neopentylthio, n-hexylthio, 2-hexylthio and 3-hexylthio.

In the context of the invention, (C₁-C₆)-alkylsulphonyl represents a straight-chain or branched alkyl radical having 1 to 6 carbon atoms which is attached via a sulphonyl group [—S(═O)₂—] to the remainder of the molecule. The following may be mentioned by way of example and by way of preference: methylsulphonyl, ethylsulphonyl, n-propylsulphonyl, isopropylsulphonyl, n-butylsulphonyl, isobutylsulphonyl, sec-butylsulphonyl, tert-butylsulphonyl, n-pentylsulphonyl and n-hexylsulphonyl.

In the context of the invention, (C₁-C₄)-alkylcarbonyl represents a straight-chain or branched alkyl radical having 1 to 4 carbon atoms which is attached to the remainder of the molecule via a carbonyl group [—C(═O)—]. The following may be mentioned by way of example and by way of preference: acetyl, propionyl, n-butyryl, isobutyryl, n-pentanoyl and pivaloyl.

In the context of the invention, (C₁-C₄)-alkylcarbonyloxy represents an oxy radical with a straight-chain or branched alkylcarbonyl substituent which contains 1 to 4 carbon atoms in the alkyl radical and is attached to the oxygen atom via the carbonyl group. The following may be mentioned by way of example and by way of preference: acetoxy, propionoxy, n-butyroxy, isobutyroxy, n-pentanoyloxy and pivaloyloxy.

In the context of the invention, (C₁-C₆)-alkoxy, (C₂-C₆)-alkoxy, (C₁-C₄)-alkoxy and (C₂-C₄)-alkoxy represent a straight-chain or branched alkoxy radical having 1 to 6, 2 to 6, 1 to 4 and 2 to 4 carbon atoms, respectively. The following may be mentioned by way of example and by way of preference: methoxy, ethoxy, n-propoxy, isopropoxy, n-butoxy, isobutoxy, sec-butoxy, tert-butoxy, n-pentoxy, 2-pentoxy, 3-pentoxy, neopentoxy, n-hexoxy, 2-hexoxy and 3-hexoxy.

In the context of the invention, (C₁-C₄)-alkoxycarbonyl represents a straight-chain or branched alkoxy radical having 1 to 4 carbon atoms which is attached to the remainder of the molecule via a carbonyl group [—C(═O)—] bonded to the oxygen atom. The following may be mentioned by way of example and by way of preference: methoxycarbonyl, ethoxycarbonyl, n-propoxycarbonyl, isopropoxycarbonyl, n-butoxycarbonyl and tert-butoxycarbonyl.

In the context of the invention, (C₃-C₆)-cycloalkyl represents a monocyclic saturated cycloalkyl group having 3 to 6 ring carbon atoms. The following may be mentioned by way of example and by way of preference: cyclopropyl, cyclobutyl, cyclopentyl and cyclohexyl.

In the context of the invention, 4- to 6-membered heterocyclyl in the definition of the radical R^{p} represents a monocyclic saturated heterocycle which has a total of 4 to 6 ring atoms, contains one or two ring heteroatoms from the group of N, O, S and/or S(O)₂ and is attached to the remainder of the molecule via a ring carbon atom or, where appropriate, a ring nitrogen atom. Preference is given to 4- or 5-membered heterocyclyl having a ring heteroatom from the group consisting of N and O and to 6-membered heterocyclyl having one or two ring heteroatoms from the group consisting of N and/or O. The following may be mentioned by way of example: azetidinyl, oxetanyl, thietanyl, pyrrolidinyl, pyrazolidinyl, tetrahydrofuranyl, thiolanyl, 1,1-dioxidothiolanyl, 1,2-oxazolidinyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, morpholinyl, thiomorpholinyl and 1,1-dioxidothiomorpholinyl. Preference is given to azetidinyl, oxetanyl, pyrrolidinyl, tetrahydrofuranyl, piperidinyl, piperazinyl, tetrahydropyranyl and morpholinyl.

A 4- to 6-membered heterocycle in the definition of the radicals R¹ and R² represents a monocyclic saturated heterocycle having a total of 4 to 6 ring atoms which contains a ring nitrogen atom through which it is attached to the remainder of the molecule and which may additionally contain a second ring heteroatom from the group consisting of N, O, S and S(O)₂. Preference is given to a 4- to 6-membered heterocycle which, in addition to the ring nitrogen atom through which it is attached, may contain a second ring heteroatom from the group consisting of N and O. The following may be mentioned by way of example: azetidin-1-yl, pyrrolidin-1-yl, pyrazolidin-1-yl, 1,2-oxazolidin-2-yl, 1,3-oxazolidin-3-yl, 1,3-thiazolidin-3-yl, piperidin-1-yl, piperazin-1-yl, morpholin-4-yl, thiomorpholin-4-yl and 1,1-dioxidothiomorpholin-4-yl. Preference is given to azetidin-1-yl, pyrrolidin-1-yl, 1,2-oxazolidin-2-yl, piperidin-1-yl, piperazin-1-yl and morpholin-4-yl.

In the context of the invention, halogen includes fluorine, chlorine, bromine and iodine. Chlorine, fluorine or bromine are preferred, and fluorine or chlorine are particularly preferred.

In the context of the present invention, all radicals which occur more than once are defined independently of one another. If radicals in the compounds according to the invention are substituted, the radicals may be mono- or polysubstituted, unless specified otherwise. Substitution by one or two identical or different substituents is preferred. Particular preference is given to substitution by one substituent.

In the context of the present invention, preference is given to compounds of the formula (I) in which
either (i)
   A represents C—R^{A}
   and
   D represents CH or N
or (ii)
   A represents CH or N
   and
   D represents C—R^{D},
where R^{A} represents chlorine, cyano, amino, (C₁-C₄)-alkyl, (C₁-C₄)-alkoxy or mono-(C₁-C₄)-alkylamino,
   where (C₁-C₄)-alkyl, (C₁-C₄)-alkoxy and mono-(C₁-C₄)-alkylamino may be substituted by hydroxy or up to three times by fluorine,
   and
   R^{D} represents (C₁-C₆)-alkyl, (C₃-C₆)-cycloalkyl or a group of the formula —NR¹R² or —C(═O)—NR³R⁴,
      where (C₁-C₆)-alkyl is substituted by hydroxy or (C₁-C₄)-alkylcarbonyloxy and may additionally be substituted up to three times by fluorine
      and
      (C₃-C₆)-cycloalkyl is substituted by hydroxy, hydroxy-(C₁-C₄)-alkyl or (C₁-C₄)-alkylcarbonyloxy,
      and where
      R¹ and R² are attached to one another and together with the nitrogen atom to which they are attached form a 4- to 6-membered heterocycle which may contain a further heteroatom from the group consisting of N(R⁵) and O and which may be substituted up to two times by identical or different radicals selected from the group consisting of fluorine, cyano, (C₁-C₄)-alkyl, hydroxy, methoxy and ethoxy,
         where (C₁-C₄)-alkyl for its part may be substituted by hydroxy or up to three times by fluorine
         and where
         R⁵ represents (C₁-C₄)-alkyl which may be substituted up to three times by fluorine or represents (C₃-C₆)-cycloalkyl, (C₁-C₄)-alkylcarbonyl or (C₁-C₄)-alkoxycarbonyl,
      and
      R³ and R⁴ independently of one another represent hydrogen or (C₁-C₄)-alkyl which may be substituted by hydroxy, methoxy or ethoxy or up to three times by fluorine
      or
      R³ and R⁴ are attached to one another and have the meanings of R¹ and R²,
Y represents CH or N,
Z represents C—R^{m} or N, where
   R^{m} represents hydrogen or fluorine,
and
R^{p} represents cyano, pentafluorothio, tri-(C₁-C₄)-alkylsilyl, trifluoromethyl, (C₂-C₆)-alkyl, trifluoromethoxy, (C₂-C₆)-alkoxy, trifluoromethylthio, (C₂-C₆)-alkylthio, (C₃-C₆)-cycloalkyl or 4- to 6-membered heterocyclyl,
   where (C₂-C₆)-alkyl, (C₂-C₆)-alkoxy and (C₂-C₆)-alkylthio for their part may be substituted by a radical selected from the group consisting of hydroxy, methoxy and ethoxy and up to six times by fluorine
   and
   (C₃-C₆)-cycloalkyl and 4- to 6-membered heterocyclyl for their part may be substituted up to two times by identical or different radicals selected from the group consisting of fluorine, (C₁-C₄)-alkyl, trifluoromethyl, hydroxy, methoxy and ethoxy,
and their salts, solvates and solvates of the salts.

A particular embodiment of the present invention comprises compounds of the formula (I) in which
A represents C—R^{A} where
   R^{A} represents chlorine, cyano or (C₁-C₄)-alkyl which may be substituted by hydroxy or up to three times by fluorine,
D represents CH,
and
R^{p} represents pentafluorothio, trimethylsilyl, trifluoromethyl, (C₂-C₆)-alkyl, trifluoromethoxy, (C₂-C₆)-alkoxy, trifluoromethylthio, (C₂-C₆)-alkylthio or (C₃-C₆)-cycloalkyl,
   where (C₂-C₆)-alkyl, (C₂-C₆)-alkoxy and (C₂-C₆)-alkylthio for their part may be substituted by a radical selected from the group consisting of hydroxy and methoxy and up to six times by fluorine
   and
   (C₃-C₆)-cycloalkyl may be substituted by a radical selected from the group consisting of fluorine, methyl, trifluoromethyl, hydroxy and methoxy,
and their salts, solvates and solvates of the salts.

A further particular embodiment of the present invention comprises compounds of the formula (I) in which
A represents C—R^{A} where
   R^{A} represents amino or mono-(C₁-C₄)-alkylamino which may be substituted by hydroxy or up to three times by fluorine,
and
D represents N,
and their salts, solvates and solvates of the salts.

A further particular embodiment of the present invention comprises compounds of the formula (I) in which
A represents CH or N,
and
D represents C—R^{D} where
   R^{D} represents (C₁-C₆)-alkyl, cyclopropyl, cyclobutyl or a group of the formula —NR¹R² or —C(═O)—NR³R⁴,
      where (C₁-C₆)-alkyl is substituted by hydroxy or acetoxy and may additionally be substituted up to three times by fluorine
      and
      cyclopropyl and cyclobutyl are substituted by hydroxy, hydroxy-(C₁-C₄)-alkyl or acetoxy,
      and where
      R¹ and R² are attached to one another and together with the nitrogen atom to which they are attached form a 4- to 6-membered heterocycle which may contain a further heteroatom from the group consisting of N(R⁵) and O and which may be substituted up to two times by identical or different radicals selected from the group consisting of fluorine, cyano, methyl, hydroxy and methoxy, where
         R⁵ represents (C₁-C₄)-alkyl which may be substituted up to three times by fluorine or represents cyclopropyl, cyclobutyl or acetyl,
      and
      R³ and R⁴ independently of one another represent hydrogen or (C₁-C₄)-alkyl which may be substituted by hydroxy, methoxy or up to three times by fluorine
      or
      R³ and R⁴ are attached to one another and have the meanings of R¹ and R²,
and their salts, solvates and solvates of the salts.

A further particular embodiment of the present invention comprises compounds of the formula (I) in which
Y represents CH
and
R^{p} represents pentafluorothio, trimethylsilyl, trifluoromethyl, (C₂-C₆)-alkyl, trifluoromethoxy, (C₂-C₆)-alkoxy, trifluoromethylthio, (C₂-C₆)-alkylthio or (C₃-C₆)-cycloalkyl,
   where (C₂-C₆)-alkyl, (C₂-C₆)-alkoxy and (C₂-C₆)-alkylthio for their part may be substituted by a radical selected from the group consisting of hydroxy and methoxy and up to six times by fluorine
   and
   (C₃-C₆)-cycloalkyl may be substituted by a radical selected from the group consisting of fluorine, methyl, trifluoromethyl, hydroxy and methoxy,
and their salts, solvates and solvates of the salts.

A further particular embodiment of the present invention comprises compounds of the formula (I) in which
Y represents N
and
R^{p} represents pentafluorothio, trimethylsilyl, trifluoromethyl, (C₂-C₆)-alkyl, trifluoromethoxy, (C₂-C₆)-alkoxy, trifluoromethylthio, (C₂-C₆)-alkylthio or (C₃-C₆)-cycloalkyl,
   where (C₂-C₆)-alkyl, (C₂-C₆)-alkoxy and (C₂-C₆)-alkylthio for their part may be substituted by a radical selected from the group consisting of hydroxy and methoxy and up to six times by fluorine
   and
   (C₃-C₆)-cycloalkyl may be substituted by a radical selected from the group consisting of fluorine, methyl, trifluoromethyl, hydroxy and methoxy,
and their salts, solvates and solvates of the salts.

A further particular embodiment of the present invention comprises compounds of the formula (I) in which
Z represents CH
and
R^{p} represents pentafluorothio, trimethylsilyl, trifluoromethyl, (C₂-C₆)-alkyl, trifluoromethoxy, (C₂-C₆)-alkoxy, trifluoromethylthio, (C₂-C₆)-alkylthio or (C₃-C₆)-cycloalkyl,
   where (C₂-C₆)-alkyl, (C₂-C₆)-alkoxy and (C₂-C₆)-alkylthio for their part may be substituted by a radical selected from the group consisting of hydroxy and methoxy and up to six times by fluorine
   and
   (C₃-C₆)-cycloalkyl may be substituted by a radical selected from the group consisting of fluorine, methyl, trifluoromethyl, hydroxy and methoxy,
and their salts, solvates and solvates of the salts.

In the context of the present invention, particular preference is given to compounds of the formula (I) in which
either (i)
   A represents C—R^{A}
   and
   D represents CH or N
or (ii)
   A represents CH or N
   and
   D represents C—R^{D},
where R^{A} represents (C₁-C₄)-alkyl, amino or mono-(C₁-C₄)-alkylamino,
   where (C₁-C₄)-alkyl and mono-(C₁-C₄)-alkylamino may be substituted by hydroxy or up to three times by fluorine,
   and
   R^{D} represents (C₁-C₄)-alkyl, cyclopropyl, cyclobutyl or a group of the formula —NR¹R² or —C(═O)—NR³R⁴,
      where (C₁-C₄)-alkyl is substituted by hydroxy or acetoxy and may additionally be substituted up to three times by fluorine
      and
      cyclopropyl and cyclobutyl are substituted by hydroxy, hydroxymethyl or acetoxy,
      and where
      R¹ and R² are attached to one another and together with the nitrogen atom to which they are attached form a 4- to 6-membered heterocycle which may contain a further heteroatom from the group consisting of N(R⁵) and O and which may be substituted by a radical selected from the group consisting of cyano, methyl, hydroxy and methoxy, where
         R⁵ represents (C₁-C₄)-alkyl which may be substituted up to three times by fluorine or represents cyclopropyl, cyclobutyl or acetyl,
      and
      R³ and R⁴ independently of one another represent hydrogen or (C₁-C₄)-alkyl which may be substituted by hydroxy
      or
      R³ and R⁴ are attached to one another and have the meanings of R¹ and R²,
Y represents CH or N,
Z represents C—R^{m} or N where
   R^{m} represents hydrogen or fluorine,
and
R^{p} represents pentafluorothio, trimethylsilyl, (C₂-C₄)-alkyl, trifluoromethoxy, (C₂-C₄)-alkoxy, trifluoromethylthio, (C₂-C₄)-alkylthio, cyclopropyl or cyclobutyl,
   where (C₂-C₄)-alkyl, (C₂-C₄)-alkoxy and (C₂-C₄)-alkylthio may be substituted by hydroxy or methoxy or up to three times by fluorine,
   and
   cyclopropyl and cyclobutyl may be substituted by a radical selected from the group consisting of fluorine, methyl, trifluoromethyl, hydroxy and methoxy,
and their salts, solvates and solvates of the salts.

In the context of the present invention, very particular preference is given to compounds of the formula (I) in which
either (i)
   A represents C—R^{A}
   and
   D represents CH or N
or (ii)
   A represents CH or N
   and
   D represents C—R^{D},
where R^{A} represents methyl, amino, methylamino or ethylamino,
   and
   R^{D} represents (C₁-C₄)-alkyl which is substituted by hydroxy or acetoxy or represents cyclopropyl which is substituted by hydroxy, hydroxymethyl or acetoxy or represents a group of the formula —NR¹R² or —C(═O)—NR³R⁴ where
      R¹ and R² are attached to one another and together with the nitrogen atom to which they are attached form a 4- to 6-membered heterocycle which may contain a further heteroatom from the group consisting of N(R⁵) and O and which may be substituted by a radical selected from the group consisting of cyano, methyl and hydroxy, where
         R⁵ represents methyl, ethyl, 2,2,2-trifluoroethyl, cyclopropyl or acetyl,
      and
      R³ and R⁴ independently of one another represent hydrogen or methyl
      or
      R³ and R⁴ are attached to one another and have the meanings of R¹ and R²,
Y represents CH or N,
Z represents CH
and
R^{p} represents trifluoromethoxy, trifluoromethylthio, 1,1,1-trifluoro-2-methylpropan-2-yl or 1-(trifluoromethyl)cyclopropan-1-yl,
and their salts, solvates and solvates of the salts.

The individual radical definitions specified in the particular combinations or preferred combinations of radicals are, independently of the particular combinations of the radicals specified, also replaced as desired by radical definitions of other combinations. Very particular preference is given to combinations of two or more of the abovementioned preferred ranges.

The present invention furthermore provides a process for preparing the compounds of the formula (I) according to the invention, characterized in that a pyridinone- or pyridazinonecarboxylic acid of the formula (II)

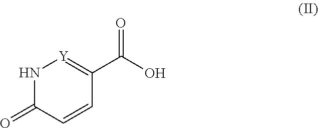

in which Y has the meaning given above
is either [a]
alkylated in the presence of a base with a compound of the formula (III)

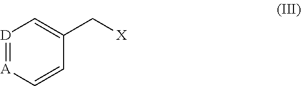

in which A and D have the meanings given above
and
X represents a customary leaving group such as, for example, chlorine, bromine, iodine, mesylate, triflate or tosylate
to give a compound of the formula (IV)

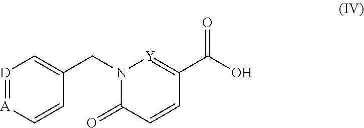

in which A, D and Y have the meanings given above
and the carboxylic acid of the formula (IV) is then condensed with an N′-hydroxyamidine of the formula (V)

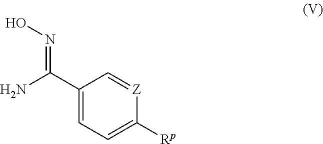

in which R^{p} and Z have the meanings given above
to give the 1,2,4-oxadiazole derivative of the formula (I) according to the invention

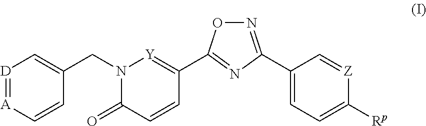

in which A, D, R^{p}, Y and Z have the meanings given above
   or is [b]
initially reacted with an N′-hydroxyamidine of the formula (V)

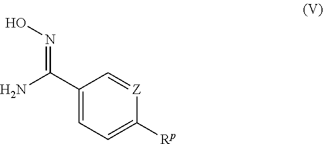

in which R^{p} and Z have the meanings given above
to give a 1,2,4-oxadiazole derivative of the formula (VI)

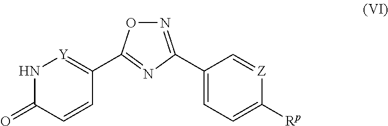

in which R^{p}, Y and Z have the meanings given above
and this is then alkylated in the presence of a base with a compound of the formula (III)

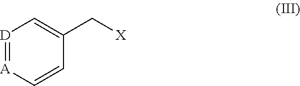

in which A and D have the meanings given above
and
X represents a customary leaving group such as, for example, chlorine, bromine, iodine, mesylate, triflate or tosylate
to give the compound of the formula (I) according to the invention

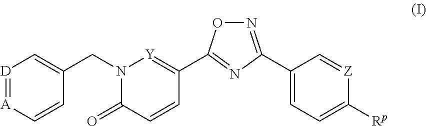

in which A, D, R^{p}, Y and Z have the meanings given above
   and the resulting compounds of the formula (I) are optionally separated into the enantiomers and/or diastereomers thereof and/or converted using the appropriate (i) solvents and/or (ii) bases or acids to the solvates, salts and/or solvates of the salts thereof.

Inert solvents for the process steps (II)+(III)→(IV) and (VI)+(III)→(I) are, for example, alcohols such as methanol, ethanol, n-propanol, isopropanol, n-butanol or tert-butanol, ethers such as diethyl ether, diisopropyl ether, methyl tert-butyl ether, tetrahydrofuran, 1,4-dioxane, 1,2-dimethoxyethane or bis-(2-methoxyethyl)ether, hydrocarbons such as benzene, toluene, xylene, ethylbenzene, pentane, hexane, cyclohexane or mineral oil fractions, or dipolar aprotic solvents such as N,N-dimethylformamide (DMF), N,N-dimethylacetamide (DMA), dimethyl sulphoxide (DMSO), N,N′-dimethylpropyleneurea (DMPU) or N-methylpyrrolidinone (NMP). It is also possible to use mixtures of these solvents. Preference is given to using methanol, tetrahydrofuran or N,N-dimethylformamide.

Suitable bases for the process steps (II)+(III)→(IV) and (VI)+(III)→(I) are customary inorganic or organic bases. These include in particular alkali metal hydroxides such as lithium hydroxide, sodium hydroxide or potassium hydroxide, alkali metal alkoxides such as sodium methoxide or potassium methoxide, sodium ethoxide or potassium ethoxide or sodium tert-butoxide or potassium tert-butoxide, alkali metal hydrides such as sodium hydride or potassium hydride, or alkali metal amides such as sodium amide, lithium diisopropylamide or lithium bis(trimethylsilyl)amide, sodium bis(trimethylsilyl)amide or potassium bis(trimethylsilyl)amide. Preference is given to using potassium hydroxide, potassium tert-butoxide or sodium hydride. If appropriate, the addition of an alkylation catalyst, for example lithium bromide, sodium iodide, potassium iodide, tetra-n-butylammonium bromide or benzyltriethylammonium chloride, may be advantageous. The reactions are generally carried out in a temperature range of from −20° C. to +120° C., preferably at from 0° C. to +70° C. The reactions can be carried out at atmospheric, elevated or reduced pressure (for example in the range from 0.5 to 5 bar); in general, the reactions are carried out at atmospheric pressure.

Suitable condensing agents for the process steps (IV)+(V)→(I) and (II)+(V)→(VI) are carbodiimides such as, for example, N,N′-diethyl-, N,N′-dipropyl-, N,N′-diisopropyl-, N,N′-dicyclohexylcarbodiimide (DCC) or N-(3-dimethylaminopropyl)-N′-ethylcarbodiimide hydrochloride (EDC), phosgene derivatives such as N,N′-carbonyldiimidazole (CDI) or isobutyl chloroformate, 1,2-oxazolium compounds such as 2-ethyl-5-phenyl-1,2-oxazolium 3-sulphate or 2-tert-butyl-5-methylisoxazolium perchlorate, acylamino compounds such as 2-ethoxy-1-ethoxycarbonyl-1,2-dihydroquinoline, α-chloroenamines such as 1-chloro-2-methyl-1-dimethylamino-1-propene, phosphorus compounds such as propanephosphonic anhydride, diethyl cyanophosphonate, bis(2-oxo-3-oxazolidinyl)phosphoryl chloride, benzotriazol-1-yloxytris(dimethylamino)phosphonium hexafluorophosphate (BOP) or benzotriazol-1-yloxytris(pyrrolidino)phosphonium hexafluorophosphate (PyBOP), or uronium compounds such as O-(benzotriazol-1-yl)-N,N,N′,N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or I-(1H-6-chlorobenzotriazol-1-yl)-1,1,3,3-tetramethyluronium tetrafluoroborate (TCTU), optionally in combination with an active ester component such as 1-hydroxy-1H-benzotriazole (HOBt), N-hydroxysuccinimide (HOSu), 4-nitrophenol or pentafluorophenol, and also as base an alkali metal carbonate, for example sodium carbonate or potassium carbonate, or tertiary amines such as triethylamine, N-methylmorpholine, N-methylpiperidine, N,N-diisopropylethylamine, pyridine or 4-N,N-dimethylaminopyridine. Preference is given to using N-(3-dimethylaminopropyl)-N′-ethylcarbodiimide hydrochloride (EDC) in combination with 1-hydroxy-1H-benzotriazole (HOBt).

The condensations (IV)+(V)→(I) and (II)+(V)→(VI) are preferably carried out in a high-boiling dipolar aprotic solvent such as, for example, N,N-dimethylformamide or dimethyl sulphoxide. The initial coupling step of these reactions is generally carried out in a temperature range of from 0° C. to +50° C.; the cyclization to the 1,2,4-oxadiazole is then accomplished by subsequent heating of the reaction mixture at temperatures of from +100° C. to +150° C. The reactions can be performed at atmospheric, elevated or reduced pressure (for example from 0.5 to 5 bar); in general, the reactions are carried out at atmospheric pressure.

Further compounds of the formula (I) according to the invention can, if expedient, also be prepared by transformation of functional groups of individual radicals or substituents, in particular those listed under R^{A}, R^{D} and R^{p}, using, as starting materials, other compounds of the formula (I) or precursors thereof obtained by the above processes. These transformations are carried out by customary methods familiar to the person skilled in the art and include, for example, reactions such as nucleophilic or electrophilic substitution reactions, transition-metal-mediated coupling reactions (e.g. Ullmann or Buchwald-Hartwig reaction), addition reactions of organometallic compounds (e.g. Grignard compounds or organolithium compounds) to carbonyl compounds, oxidations and reduction reactions, hydrogenation, alkylation, acylation, amination, the formation of nitriles, carboxylic esters and carboxamides, ester cleavage and ester hydrolysis and also the introduction and removal of temporary protective groups.

Likewise, if expedient, compounds of the formula (I) according to the invention can also be prepared by employing, in the starting materials of the process variants described above, instead of the substituents R^{A}, R^{D} and/or R^{p} initially other functional groups which do not fall into the scope of the meaning of R^{A}, R^{D} and R^{p}, which are then converted by subsequent transformations familiar to the person skilled in the art (as listed in an exemplary manner in the previous paragraph) into the respective substituents R^{A}, R^{D} and R^{p}. Examples of such functional groups which serve as “precursors” for R^{A}, R^{D} and/or R^{p} are radicals such as nitro, hydroxy, chlorine, bromine, methanesulphonate (mesylate), trifluoromethanesulphonate (triflate), formyl, alkylcarbonyl, hydroxycarbonyl and alkoxycarbonyl [cf. also the preparation of the working examples and their precursors described in detail in the experimental part below].

The compounds of the formulae (II), (III) and (V) are commercially available or described as such in the literature, or they can be prepared in a way obvious to the person skilled in the art, in analogy to methods published in the literature. Numerous detailed instructions and literature information for the preparation of the starting materials are also to be found in the experimental part in the section on the preparation of the starting compounds and intermediates.

The preparation of the compounds according to the invention can be illustrated in an exemplary manner by the reaction schemes below:

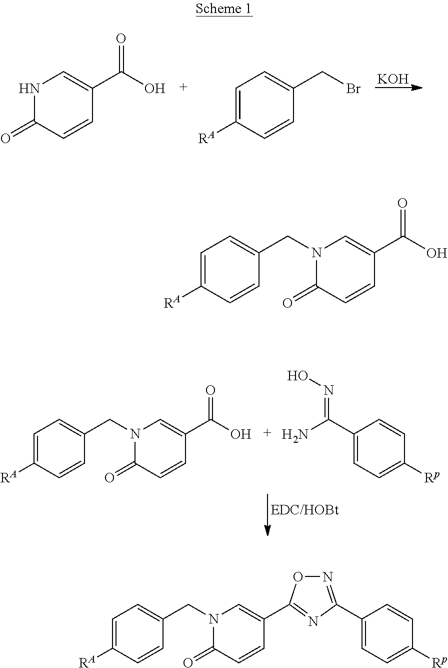

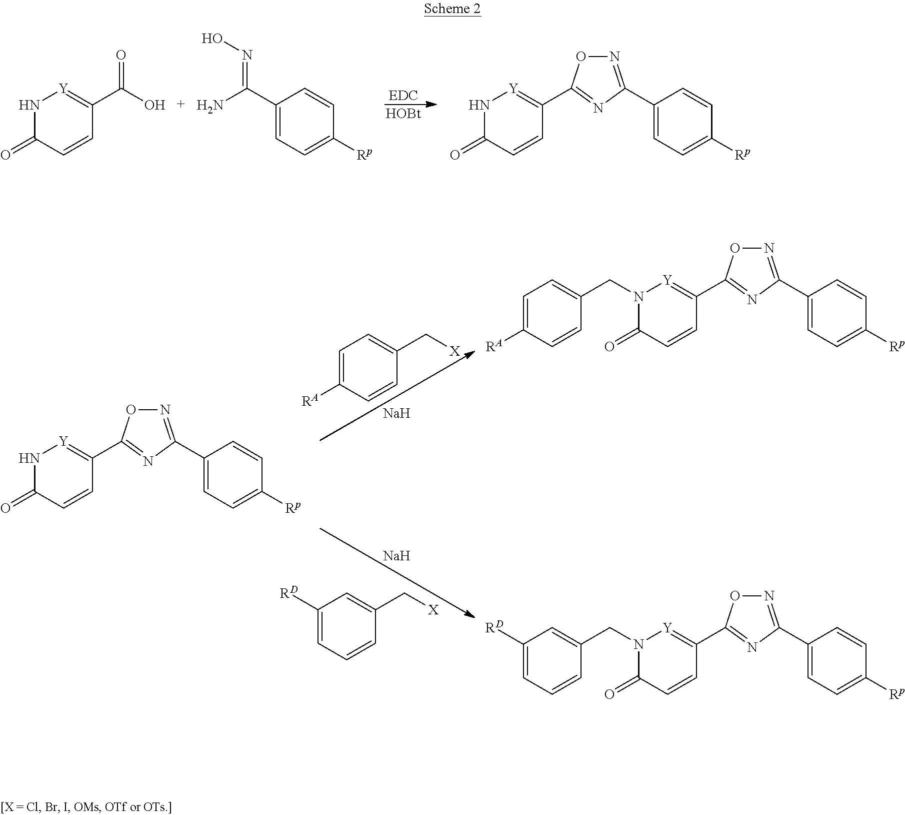

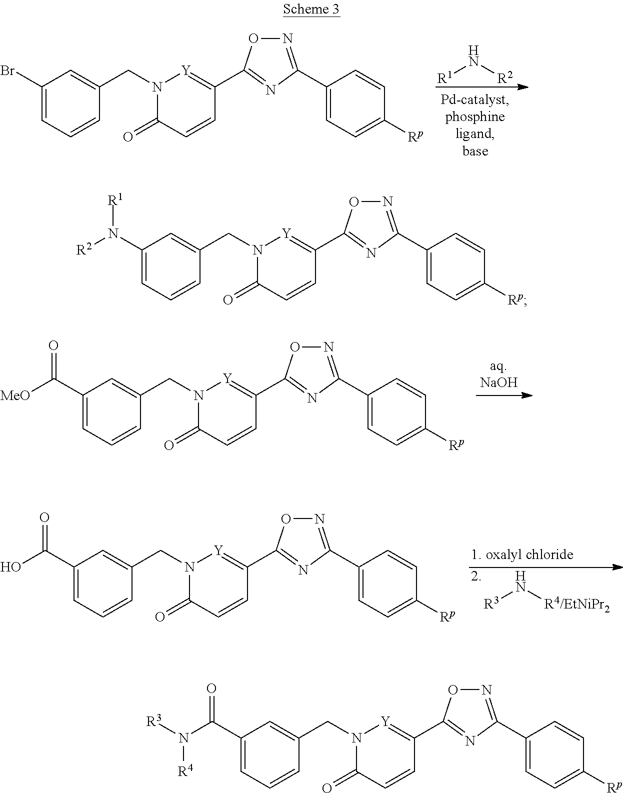

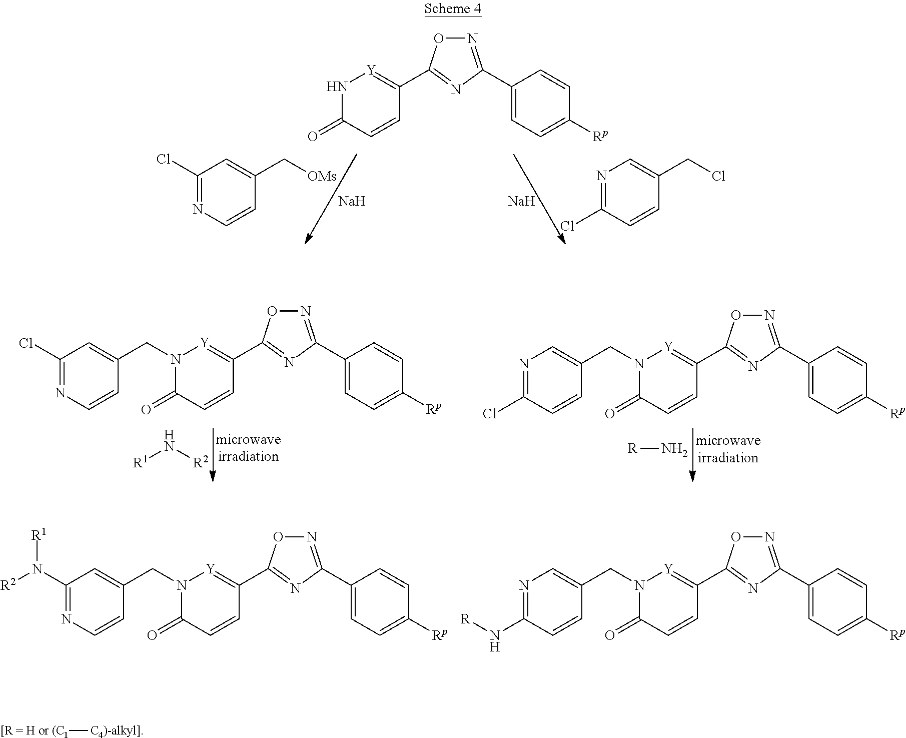

The compounds according to the invention have valuable pharmacological properties and can be used for prevention and treatment of diseases in humans and animals.

In the context of the present invention, the term “treatment” or “treating” includes controlling, inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of experiencing, suffering from, contracting or having a disease, a condition, a disorder, an injury or a health problem, or a development or progression of such states and/or the symptoms of such states.

In this context, the treatment or prevention of a disease, a condition, a disorder, an injury or a health problem may be partial or complete.

The compounds according to the invention are highly potent inhibitors of the regulatory HIF pathway. In addition, the compounds according to the invention have an advantageous pharmacokinetic profile which makes them suitable for oral administration.

On the basis of their profile of action, the compounds according to the invention are especially suitable for treatment of hyperproliferative diseases in humans and in mammals in general. The compounds can inhibit, block, reduce or lower cell proliferation and cell division, and secondly increase apoptosis.

The hyperproliferative diseases for the treatment of which the compounds according to the invention can be employed include, inter alia, psoriasis, keloids, formation of scars and other proliferative diseases of the skin, benign diseases, such as benign prostate hyperplasia (BPH), and in particular the group of cancer and tumour diseases. In the context of the present invention, these are understood to mean especially the following diseases, but without any limitation thereto: mammary carcinomas and mammary tumours (ductal and lobular forms, also in situ), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, stomach, gall bladder, small intestine, large intestine, rectum), liver tumours (inter alia hepatocellular carcinoma, cholangiocarcinoma and mixed hepatocellular cholangiocarcinoma), tumours of the head and neck region (larynx, hypopharynx, nasopharynx, oropharynx, lips and oral cavity), skin tumours (squamous cell carcinoma, Kaposi's sarcoma, malignant melanoma, Merkel cell skin cancer and non-melanomatous skin cancer), tumours of soft tissue (inter alia soft tissue sarcomas, osteosarcomas, malignant fibrous histiocytomas, lymphosarcomas and rhabdomyosarcomas), tumours of the eyes (inter alia intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testes in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-described diseases in humans can also occur with a comparable aetiology in other mammals and can be treated there with the compounds of the present invention.

The compounds according to the invention act as modulators of the HIF regulation pathway and are therefore also suitable for treatment of diseases associated with a harmful expression of the HIF transcription factor. This applies especially to the transcription factors HIF-1α and HIF-2α. The term “harmful expression of HIF” here means abnormal physiological presence of HIF protein. This can be caused by excessive synthesis of the protein (mRNA- or translation-related), by reduced degradation or by inadequate counter-regulation in the functioning of the transcription factor.

HIF-1α and HIF-2α regulate more than 100 genes. This applies to proteins which play a role in angiogenesis and are therefore directly relevant to tumours, and also those which influence glucose, amino acid and lipid metabolism, and cell migration, metastasis and DNA repair, or improve the survival of tumour cells by suppressing apoptosis. Others act more indirectly via inhibition of the immune reaction and upregulation of angiogenic factors in inflammation cells. HIF also plays an important role in stem cells, and here especially tumour stem cells, which are reported to have elevated HIF levels. The inhibition of the HIF regulation pathway by the compounds of the present invention thus also has a therapeutic influence on tumour stem cells, which do not have a high proliferation rate and therefore are affected only inadequately by cytotoxic substances (cf. Semenza, 2007; Weidemann and Johnson, 2008).

Changes in cell metabolism by HIF are not exclusive to tumours, but also occur in other hypoxic pathophysiological processes, whether chronic or transient. HIF inhibitors—such as the compounds of the present invention—are therapeutically helpful in those connections in which, for example, additional damage arises from adaptation of cells to hypoxic situations, since damaged cells can cause further damage if they do not function as intended. One example of this is the formation of epileptic foci in partly destroyed tissue following strokes. A similar situation is found in the case of cardiovascular diseases if ischaemic processes occur in the heart or in the brain as a consequence of thromboembolic events, inflammations, wounds, intoxications or other causes. These can lead to damage such as a locally retarded action potential, which in turn can bring about arrhythmias or chronic heart failure. In transient form, for example as a result of apnoea, there may under certain circumstances be essential hypertension, which can lead to known sequelae, for example stroke and cardiac infarction.

Inhibition of the HIF regulation pathway such as is achieved by the compounds according to the invention can therefore also be helpful for diseases such as heart failure, arrhythmia, cardiac infarction, apnoea-induced hypertension, pulmonary hypertension, transplant ischaemia, reperfusion damage, stroke and macular degeneration, as well as for recovery of nerve function after traumatic damage or severance.

Since HIF is one of the factors which control the transition from an epithelial to a mesenchymal cell type, which is important especially for the lung and kidney, the compounds according to the invention can also be used to prevent or control fibroses of the lung and kidney associated with HIF.

Further diseases for the treatment of which the compounds according to the invention can be used are inflammatory joint diseases, such as various forms of arthritis, and inflammatory intestinal diseases, such as, for example, Crohn's disease.

Chuvash polycythaemia is mediated by HIF-2α activity during erythropoiesis, in the spleen among other organs. The compounds according to the invention, as inhibitors of the HIF regulation pathway, are therefore also suitable here for suppressing excessive erythrocyte formation and hence for alleviating the effects of this disease.

The compounds of the present invention can also be used for treatment of diseases associated with excessive or abnormal angiogenesis. These include, inter alia, diabetic retinopathy, ischaemic retinal vein occlusion and retinopathy in premature babies (cf. Aiello et al., 1994; Peer et al., 1995), age-related macular degeneration (AMD; cf. Lopex et al., 1996), neovascular glaucoma, psoriasis, retrolental fibroplasia, angiofibroma, inflammation, rheumatic arthritis (RA), restenosis, in-stent restenosis and restenosis following vessel implantation.

Increased blood supply is additionally associated with cancerous, neoplastic tissue and leads here to accelerated tumour growth. Moreover, the growth of new blood and lymph vessels facilitates the formation of metastases and hence the spread of the tumour. New lymph and blood vessels are also harmful to allografts in immunoprivileged tissues, such as the eye, which, for example, increases susceptibility to rejection reactions. Compounds of the present invention can therefore also be used for therapy of one of the aforementioned diseases, for example by inhibition of the growth of by a reduction in the number of blood vessels. This can be achieved via inhibition of endothelial cell proliferation or other mechanisms for preventing or attenuating the formation of vessels and via a reduction of neoplastic cells by apoptosis.

In the case of obesity, HIF-1α becomes enriched in the adipose tissue, resulting in a HIF-mediated shift in the catabolism in the direction of glycolysis, such that an increased amount of glucose as an energy carrier is consumed. This leads at the same time to reduced lipid metabolism and hence to storage of lipids in the tissue. The substances according to the invention are therefore also suitable for treatment of HIF-1α-mediated enrichment of lipids in the tissue, especially in the case of obesity.

The literature describes that HIF regulates cellular processes in β-cells, liver and muscle cells and in fat cells of adipose tissue; HIF also has an effect on body weight control and plays a role in particular in Type II diabetes (cf. Girgis et al., 2012). Accordingly, the compounds according to the invention are also suitable for the treatment of diabetes, metabolic syndrome and pathological overweight (obesity).

The present invention thus further provides for the use of the compounds according to the invention for the treatment and/or prophylaxis of disorders, in particular the disorders mentioned above.

The present invention further provides for the use of the compounds according to the invention for producing a medicament for treatment and/or prophylaxis of disorders, in particular the disorders mentioned above.

The present invention furthermore provides the use of the compounds according to the invention in a method for treatment and/or prevention of disorders, in particular the abovementioned disorders.

The present invention further provides a method for treatment and/or prophylaxis of disorders, in particular the disorders mentioned above, using an effective amount of at least one of the compounds according to the invention.

The compounds according to the invention can be used alone or, if required, in combination with one or more other pharmacologically active substances, provided that this combination does not lead to undesirable and unacceptable side effects. The present invention furthermore therefore provides medicaments containing at least one of the compounds according to the invention and one or more further active compounds, in particular for treatment and/or prevention of the abovementioned disorders.

For example, the compounds of the present invention can be combined with known antihyperproliferative, cytostatic or cytotoxic substances for the treatment of cancer diseases. The combination of the compounds according to the invention with other substances customary for cancer therapy or also with radiotherapy is therefore indicated in particular, since hypoxic regions of a tumour respond only weakly to the conventional therapies mentioned, whereas the compounds of the present invention display their activity there in particular.

Examples of suitable combination active compounds include:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, busulphan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethynylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, streptozocin, strontium-89 chloride, synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulphan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, regorafenib, 13-cis-retic acid, satraplatin, seocalcitol, Sorafenib, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulphan, camptothecin, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenine, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins, which additively or synergistically intensify the effects of inhibition of the HIF signal pathway transmission.

Inhibitors of the HIF regulation pathway, such as the compounds according to the invention, can also achieve positive effects in combination with other therapies directed against angiogenesis, for example with avastin, axitinib, recentin, regorafenib, sorafenib or sunitinib. Combinations with inhibitors of the proteasome and of mTOR, and also with antihormones and steroidal metabolic enzyme inhibitors, are likewise particularly suitable because of their favourable profile of side effects.

Generally, the following aims can be pursued with the combination of compounds of the present invention with other cytostatically or cytotoxically active agents:
improved efficacy in slowing the growth of a tumour, in reducing its size or even in the complete elimination thereof, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of tumours;
the achievement of a higher rate of response to the therapy;
a longer survival time of the patient compared with present-day standard therapy.

In addition, the compounds according to the invention can also be used in conjunction with radiotherapy and/or surgical intervention.

The present invention further provides medicaments which comprise at least one compound according to the invention, typically together with one or more inert, nontoxic, pharmaceutically suitable excipients, and the use thereof for the aforementioned purposes.

The compounds according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds according to the invention can be administered in administration forms suitable for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example with enteric coatings or coatings that dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions.

Parenteral administration can bypass an absorption step (e.g. intravenously, intraarterially, intracardially, intraspinally or intralumbally) or include an absorption (e.g. intramuscularly, subcutaneously, intracutaneously, percutaneously or intraperitoneally). Suitable administration forms for parenteral administration include injection and infusion formulations in the form of solutions, suspensions, emulsions, lyophilisates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral and parenteral administration are preferred, especially oral and intravenous administration.

The compounds according to the invention can be converted to the administration forms mentioned. This can be done in a manner known per se, by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration, the dosage is about 0.01 to 100 mg/kg, preferably about 0.01 to 20 mg/kg and most preferably 0.1 to 10 mg/kg of body weight.

It may nevertheless be necessary where appropriate to deviate from the stated amounts, specifically as a function of the body weight, route of administration, individual response to the active compound, nature of the preparation and time or interval over which administration takes place. For instance, in some cases, less than the aforementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not limited to the examples.

The percentages in the tests and examples which follow are, unless indicated otherwise, percentages by weight; parts are parts by weight. Solvent ratios, dilution ratios and concentration figures for liquid/liquid solutions are each based on volume.

### A. EXAMPLES

### Abbreviations and Acronyms

abs. absolute
Ac acetyl
aq. aqueous, aqueous solution
br. broad (in NMR)
Ex. Example
Bu butyl
CI chemical ionization (in MS)
d doublet (in NMR)
d day
dba dibenzylideneacetone
TLC thin-layer chromatography
DCI direct chemical ionization (in MS)
dd doublet of doublets (in NMR)
DMAP 4-N,N-dimethylaminopyridine
DME 1,2-dimethoxyethane
DMF N,N-dimethylformamide
DMSO dimethyl sulphoxide
dt doublet of triplets (in NMR)
EDC N′-(3-dimethylaminopropyl)-N-ethylcarbodiimide hydrochloride
EI electron impact ionization (in MS)
eq. equivalent
ESI electrospray ionization (in MS)
Et ethyl
GC gas chromatography
GC/MS gas chromatography-coupled mass spectrometry
h hour(s)
HOBt 1-hydroxy-1H-benzotriazole hydrate
HPLC high-pressure, high-performance liquid chromatography
ⁱPr isopropyl
LC/MS liquid chromatography-coupled mass spectrometry
LDA lithium diisopropylamide
LiHMDS lithium hexamethyldisilazide
lit. literature (reference)
m multiplet (in NMR)
Me methyl
min minute(s)
MPLC medium-pressure liquid chromatography (on silica gel; also referred to as flash chromatography)
Ms methanesulphonyl (mesyl)
MS mass spectrometry
NMP N-methyl-2-pyrrolidinone
NMR nuclear magnetic resonance spectrometry
Pd/C palladium on activated carbon
PEG polyethylene glycol
Pr propyl
q or quart quartet (in NMR)
quint quintet (in NMR)
R_{f} retention index (in TLC)
RT room temperature
Rₜ retention time (in HPLC)
singlet (in NMR)
sept septet (in NMR)
t triplet (in NMR)
TBAF tetra-n-butylammonium fluoride
^{t}Bu tert-butyl
Tf trifluoromethylsulphonyl (triflyl)
TFA trifluoroacetic acid
THF tetrahydrofuran
THP tetrahydro-2H-pyran-2-yl
TIPS triisopropylsilyl
Ts para-tolylsulphonyl (tosyl)
UV ultraviolet spectrometry
v/v ratio by volume (of a solution)
X-Phos 2-dicyclohexylphosphino-2′,4′,6′-triisopropylbiphenyl
tog. together

### HPLC, LC/MS and GC/MS Methods:

### Method 1 (LC/MS):

Instrument: Micromass Quattro Premier with Waters UPLC Acquity; column: Thermo Hypersil GOLD 1.9 μm, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; temperature: 50° C.; UV detection: 210 nm.

### Method 2 (LC/MS):

Instrument: Micromass Quattro Premier with Waters UPLC Acquity; column: Thermo Hypersil GOLD 1.9 μm, 50 mm×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; flow rate: 0.3 ml/min; temperature: 50° C.; UV detection: 210 nm.

### Method 3 (LC/MS):

Instrument: Waters Acquity SQD UPLC System; column: Waters Acquity UPLC HSS T3 1.8 μm, 50 mm×1 mm; mobile phase A: 1 l of water+0.25 ml of 99% strength formic acid, mobile phase B: 1 l of acetonitrile+0.25 ml of 99% formic acid; gradient: 0.0 min 90% A→1.2 min 5% A→2.0 min 5% A; flow rate: 0.40 ml/min; temperature: 50° C.; UV detection: 210-400 nm.

### Method 4 (LC/MS):

MS instrument type: Micromass Quattro Micro; HPLC instrument type: Agilent series 1100; column: Thermo Hypersil GOLD 3 μm, 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A; temperature: 50° C.; flow rate: 2 ml/min; UV detection: 210 nm.

### Method 5 (LC/MS):

MS instrument type: Micromass ZQ; apparatus type HPLC: HP 1100 series; UV DAD; column: Phenomenex Gemini 3 μm, 30 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; temperature: 50° C.; UV detection: 210 nm.

### Method 6 (LC/MS):

MS instrument type: Micromass ZQ; apparatus type HPLC: Waters Alliance 2795; column: Phenomenex Synergi 2.5 μm, MAX-RP 100A Mercury 20 mm×4 mm; mobile phase A: 1 lof water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; temperature: 50° C.; UV detection: 210 nm.

### Method 7 (LC/MS):

Instrument: Waters Acquity SQD UPLC System; column: Waters Acquity UPLC HSS T3 1.8 μm, 30 mm×2 mm; mobile phase A: 1 l of water+0.25 ml of 99% strength formic acid, mobile phase B: 1 l of acetonitrile+0.25 ml of 99% formic acid; gradient: 0.0 min 90% A→1.2 min 5% A→2.0 min 5% A; flow rate: 0.60 ml/min; temperature: 50° C.; UV detection: 208-400 nm.

### Method 8 (LC/MS):

Instrument: Waters Acquity SQD UPLC System; column: Waters Acquity UPLC HSS T3 1.8 μm, 50 mm×1 mm; mobile phase A: 1 l of water+0.25 ml of 99% strength formic acid, mobile phase B: 1 l of acetonitrile+0.25 ml of 99% formic acid; gradient: 0.0 min 95% A→6.0 min 5% A→7.5 min 5% A; flow rate: 0.35 ml/min; temperature: 50° C.; UV detection: 210-400 nm.

### Method 9 (GC/MS):

Instrument: Micromass GCT, GC 6890; column: Restek RTX-35, 15 m×200 μm×0.33 μm; constant helium flow rate: 0.88 ml/min; oven: 70° C.; inlet: 250° C.; gradient: 70° C., 30° C./min→310° C. (maintained for 3 min).

### Method 10 (Preparative HPLC):

column: Sunfire C18, 5 μm, 75 mm×30 mm; sample dissolved in 20 ml of DMSO+10 ml of methanol; injection volume: 2.0 ml; mobile phase: methanol/water/1% aq. formic acid; gradient: 0.00-1.00 min 30:65:5→9.00-10.50 min 95:0:5; flow rate: 60 ml/min

### Method 11 (Preparative HPLC):

column: Sunfire C18, 5 μm, 250 mm×20 mm; sample dissolved in 3 ml of methanol+1 ml of water; injection volume: 1.0 ml; mobile phase: methanol/water 75:25, 0-8 min; flow rate: 25 ml/min

### Method 12 (Preparative HPLC):

column: Sunfire C18, 5 μm, 250 mm×20 mm; sample dissolved in 5 ml of methanol+3 ml of 0.2% aq. TFA; injection volume: 1.0 ml; mobile phase: methanol/0.2% aq. TFA 75:25, 0-10 min; flow rate: 25 ml/min; temperature: 25° C.; UV detection: 210 nm.

### Method 13 (Preparative HPLC):

column: YMC-ODS-AQ, C18, 10 μm, 250 mm×30 mm; mobile phase: methanol/0.1% aq. TFA; gradient: 40:60 (0.00-4.25 min)→60:40 (4.25-4.50 min)→80:20 (4.50-11.50 min)→100:0 (11.50-12.00 min)→100:0 (12.00-14.50 min)→40:60 (14.50-14.75 min)→40:60 (14.75-18.00 min).

### Method 14 (Preparative HPLC):

column: YMC-ODS-AQ, C18, 10 μm, 250 mm×30 mm; mobile phase: methanol/0.1% aq. TFA; gradient: 50:50 (0.00-4.25 min)→70:30 (4.25-4.50 min)→90:10 (4.50-11.50 min)→100:0 (11.50-12.00 min)→100:0 (12.00-14.50 min)→50:50 (14.50-14.75 min)→50:50 (14.75-18.00 min).

### Method 15 (Preparative HPLC):

column: YMC-ODS-AQ, C18, 10 μm, 250 mm×30 mm; mobile phase: methanol/0.1% aq. TFA; gradient: 60:40 (0.00-4.25 min)→80:20 (4.25-4.50 min)→100:0 (4.50-11.50 min)→100:0 (11.50-14.50 min)→60:40 (14.50-14.75 min)→60:40 (14.75-18.00 min).

### Method 16 (Preparative HPLC):

column: Reprosil-Pur C18, 10 μm, 250 mm×30 mm; mobile phase: acetonitrile/0.1% aq. formic acid; gradient: 10:90→90:10.

### Method 17 (Preparative HPLC):

column: GromSil ODS-4HE, 10 μm, 250 mm×30 mm; mobile phase: acetonitrile/0.1% aq. formic acid; gradient: 10:90→90:10.

### Method 18 (Preparative HPLC):

column: XBridge C18, 5 μm, 150 mm×19 mm; sample dissolved in 2 ml of DMSO; injection volume: 1 ml; mobile phase: acetonitrile/water/0.1% aq. formic acid; gradient: 0-1 min 20:75:5→1.25 min 45:50:5→9.5 min 65:30:5→10-12 min 95:0:5; flow rate: 25 ml/min

### Method 19 (Preparative HPLC):

column: XBridge C18, 5 μm, 150 mm×19 mm; sample dissolved in 2 ml of DMSO; injection volume: 1 ml; mobile phase: acetonitrile/water/0.1% aq. diethylamine; gradient: 0-1 min 20:75:5→1.25 min 40:55:5→9.5 min 60:35:5→10-12 min 95:0:5; flow rate: 25 ml/min

The descriptions of the coupling patterns of ¹H NMR signals below refer to the optical appearance of the signals in question and do not necessarily correspond to a strict, physically correct interpretation. In general, the stated chemical shift refers to the centre of the signal in question; in the case of broad multiplets, an interval is given.

Melting points and melting points ranges, if stated, are uncorrected.

For all the reactants or reagents for which the preparation is not described explicitly in the following, they were obtained commercially from generally accessible sources. For all the other reactants or reagents for which the preparation likewise is not described in the following and which were not commercially obtainable or were obtained from sources which are not generally accessible, reference is made to the published literature in which their preparation is described.

### Starting Materials and Intermediates

### Example 1A

### 1-(3-{[(Methylsulphonyl)oxy]methyl}phenyl)cyclopropyl acetate

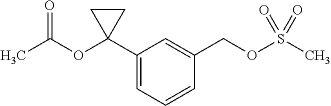 [SMILES: CC(=O)OC1(c2cccc(COS(C)(=O)=O)c2)CC1]

### Step 1: 1-[3-({[tert-Butyl(dimethyl)silyl]oxy}methyl)phenyl]cyclopropanol

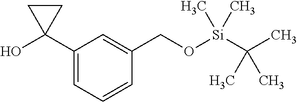 [SMILES: CC(C)(C)[Si](C)(C)OCc1cccc(C2(O)CC2)c1]

Preparation of Solution A:

60 ml of methanol and a drop of concentrated hydrochloric acid were added to 12.32 g (70.7 mmol) of [(1-ethoxycyclopropyl)oxy](trimethyl)silane, and the mixture was stirred at RT overnight. The solvent was then removed at RT and under a reduced pressure of not less than 30 mbar on a rotary evaporator. This gave 6.26 g (61.27 mmol) of 1-ethoxycyclopropanol, which were dissolved in 80 ml of THF. Under argon, this solution was then cooled to −70° C., and 30.6 ml (61.27 mmol) of a 2 M solution of ethylmagnesium chloride in THF were added. The cooling bath was then removed, and the solution was stirred without cooling until an internal temperature of 0° C. had been reached.

Preparation of Solution B:

Under argon and at −40° C., 47.1 ml (61.27 mmol) of a 1.3 M solution of isopropylmagnesium chloride/lithium chloride complex in THF were added to a solution of 19.40 g (55.70 mmol) of tert-butyl[(3-iodobenzyl)oxy]dimethylsilane in 280 ml of THF, and the mixture was stirred at −40° C. for 1 h.

Once the two solutions had been prepared, solution A was added to solution B at 0° C. The reaction mixture was then heated under reflux for 1 h. After cooling to RT, saturated aqueous ammonium chloride solution was added, and the mixture was extracted twice with tert-butyl methyl ether. The combined organic phases were washed once with saturated sodium chloride solution, dried over magnesium sulphate, filtered and concentrated. The residue was purified by means of flash chromatography (silica gel, mobile phase: cyclohexane→cyclohexane/ethyl acetate 85:15). Removal of the solvent gave 9.55 g (60% of theory) of the title compound.

¹H NMR (400 MHz, CDCl₃, δ/ppm): 7.32-7.24 (m, 2H), 7.22-7.16 (m, 2H), 4.74 (s, 2H), 2.36 (s, 1H), 1.26 (dd, 2H), 1.06 (dd, 2H), 0.94 (s, 9H), 0.11 (s, 6H).

MS (DCI, NH₃): m/z=296 [M+NH₄]⁺.

### Step 2: 1-[3-({[tert-Butyl(dimethyl)silyl]oxy}methyl)phenyl]cyclopropyl acetate

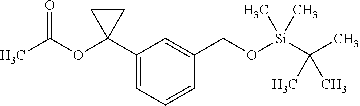 [SMILES: CC(=O)OC1(c2cccc(CO[Si](C)(C)C(C)(C)C)c2)CC1]

At RT, 21.4 ml (42.87 mmol) of a 2 M solution of ethylmagnesium chloride in THF, directly followed by 3.0 ml (42.87 mmol) of acetyl chloride, were added to a solution of 9.55 g (34.3 mmol) of the compound from Example 1A/step 1 in 100 ml of THF. After 5 min of stirring at RT, saturated aqueous ammonium chloride solution was added, and the mixture was extracted twice with ethyl acetate. The combined organic phases were dried over magnesium sulphate, filtered and concentrated. This gave 11.25 g (94% pure, 96% of theory) of the title compound.

¹H NMR (400 MHz, CDCl₃, δ/ppm): 7.30-7.24 (m, 2H), 7.20-7.13 (m, 2H), 4.72 (s, 2H), 2.04 (s, 3H), 1.31-1.25 (m, 2H), 1.24-1.18 (m, 2H), 0.94 (s, 9H), 0.09 (s, 6H).

MS (DCI, NH₃): m/z=338 [M+NH₄]⁺.

### Step 3: 1-[3-(Hydroxymethyl)phenyl]cyclopropyl acetate

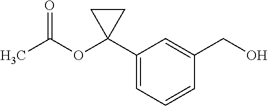

At RT, 65.6 ml (65.6 mmol) of a 1 M solution of tetra-n-butylammonium fluoride in THF were added to a solution of 11.25 g (32.82 mmol, purity 94%) of the compound from Example 1A/step 2. The mixture was stirred at RT for 30 min and then diluted with ethyl acetate and washed once with water. The aqueous phase was reextracted once with ethyl acetate. The combined organic phases were washed once with saturated sodium chloride solution, dried over magnesium sulphate and concentrated. This gave 8.0 g (80% pure, 95% of theory) of the title compound.

¹H NMR (400 MHz, CDCl₃, δ/ppm): 7.24-7.12 (m, 5H), 4.58 (s, 2H), 1.95 (s, 3H), 1.22-1.17 (m, 2H), 1.16-1.10 (m, 2H).

### Step 4: 1-(3-{[(Methylsulphonyl)oxy]methyl}phenyl)cyclopropyl acetate

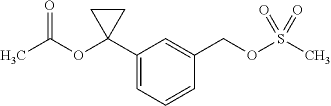 [SMILES: CC(=O)OC1(c2cccc(COS(C)(=O)=O)c2)CC1]

At 0° C., 2.8 ml (37.24 mmol) of methanesulphonyl chloride were added dropwise to a solution of 8.0 g (31.03 mmol, purity 80%) of the compound from Example 1A/step 3 and 5.6 ml (40.34 mmol) of triethylamine in 90 ml THF. The mixture was then slowly warmed to RT, stirred at RT for another 10 min and then diluted with ethyl acetate. The mixture was washed once with water, and the aqueous phase was reextracted once with ethyl acetate. The combined organic phases were washed once with saturated sodium chloride solution, dried over magnesium sulphate and concentrated. The residue obtained was purified by means of flash chromatography (silica gel, mobile phase: cyclohexane/ethyl acetate 95:5→70:30). Removal of the solvent and drying of the residue under reduced pressure gave 8.45 g (95% pure, 91% of theory) of the title compound.

¹H NMR (400 MHz, CDCl₃, δ/ppm): 7.39-7.27 (m, 4H), 5.22 (s, 2H), 2.90 (s, 3H), 2.06 (s, 3H), 1.35-1.28 (m, 2H), 1.27-1.20 (m, 2H).

LC/MS (Method 1, ESIpos): Rₜ=1.02 min; m/z=285 [M+H]⁺.

### Example 2A

### 3-(2-Hydroxy-2-methylpropyl)benzyl methanesulphonate

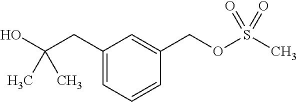 [SMILES: CC(C)(O)Cc1cccc(COS(C)(=O)=O)c1]

### Step 1: 1-(3-Bromophenyl)-2-methylpropan-2-ol

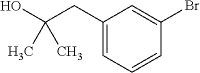

At 0° C., 55 ml (164 mmol) of a 3 M solution of methylmagnesium chloride in THF were added dropwise to a solution of 15.0 g (65.5 mmol) of methyl (3-bromophenyl)acetate in 600 ml of anhydrous THF. After the addition had ended, stirring was continued at 0° C. for 1 h. The ice/water bath was then removed, and stirring was continued at RT overnight. About 1.2 litres of saturated aqueous ammonium chloride solution were then added, and the mixture was extracted three times with about 200 ml of ethyl acetate each time. The combined organic extracts were washed with saturated sodium chloride solution, dried over anhydrous magnesium sulphate, filtered and finally freed from the solvent under reduced pressure. The residue obtained was purified by filtration with suction through silica gel using the mobile phase cyclohexane/ethyl acetate 10:1→1:1. This gave 8.04 g (53% of theory) of the title compound.

¹H NMR (400 MHz, CDCl₃, δ/ppm): 7.41-7.37 (m, 2H), 7.20-7.13 (m, 2H), 2.73 (s, 2H), 1.32 (s, 1H), 1.23 (s, 6H).

GC/MS (Method 9, EIpos): Rₜ=4.56 min, m/z=210/212 [M−H₂O]⁺.

### Step 2: 3-(2-Hydroxy-2-methylpropyl)benzaldehyde

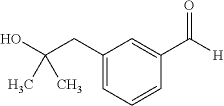

At −78° C., 13.7 ml (21.8 mmol) of an n-butyllithium solution (1.6 M in hexane) were added dropwise to a solution of 2.50 g (10.9 mmol) of the compound from Example 2A/step 1 in 100 ml of anhydrous THF. After the addition had ended, the mixture was stirred at −78° C. for another 30 min, and then, also at −78° C., 2.6 ml (32.8 mmol) of anhydrous N,N-dimethylformamide were added. The cooling bath was then removed, and stiffing was continued at RT overnight. About 100 ml of saturated aqueous ammonium chloride solution were then added, and the mixture was extracted three times with about 100 ml of ethyl acetate each time. The combined organic extracts were washed successively with water and saturated sodium chloride solution, dried over anhydrous magnesium sulphate, filtered and finally freed from the solvent under reduced pressure. The crude product obtained in this way was purified by means of MPLC (silica gel, mobile phase: cyclohexane/ethyl acetate 2:1). This gave 1.15 g (59% of theory) of the title compound.

¹H NMR (400 MHz, CDCl₃, δ/ppm): 10.01 (s, 1H), 7.79-7.74 (m, 2H), 7.53-7.47 (m, 2H), 2.86 (s, 2H), 1.25 (s, 6H).

GC/MS (Method 9, EIpos): Rₜ=4.76 min, m/z=210/160 [M−H₂O]⁺.

### Step 3: 1-[3-(Hydroxymethyl)phenyl]-2-methylpropan-2-ol

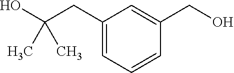

At 0° C., 6.0 ml (6.00 mmol) of a lithium aluminium hydride solution (1.0 M in THF) were added dropwise to a solution of 1.07 g (6.00 mmol) of the compound from Example 2A/step 2 in 30 ml of anhydrous THF. After the addition had ended, stirring was continued at RT for 1 h. Carefully, 1-2 ml of saturated aqueous ammonium chloride solution and then about 30 ml of ethyl acetate were then added. Anhydrous magnesium sulphate was added in the amount required to take up the aqueous phase completely. After filtration, the filtrate was freed from the solvent on a rotary evaporator and the residue was dried under high vacuum. This gave 1.09 g (100% of theory) of the title compound.

¹H NMR (400 MHz, CDCl₃, δ/ppm): 7.31 (t, 1H), 7.25 (dd, 1H), 7.22 (dd, 1H), 7.14 (dd, 1H), 4.69 (s, broad, 2H), 2.78 (s, 2H), 1.79 (broad, 1H), 1.41 (s, broad, 1H), 1.23 (s, 6H).

GC/MS (Method 9, EIpos): Rₜ=5.00 min, m/z=210/162 [M−H₂O]⁺.

### Step 4: 3-(2-Hydroxy-2-methylpropyl)benzyl methanesulphonate

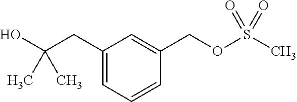 [SMILES: CC(C)(O)Cc1cccc(COS(C)(=O)=O)c1]

At 0° C., 1.12 g (6.41 mmol) of methanesulphonic anhydride were added to a solution of 1.05 g (5.83 mmol) of the compound from Example 2A/step 3 and 1.2 ml (8.74 mmol) of triethylamine in 60 ml of anhydrous dichloromethane. The mixture was then stirred at RT for another 1 h. The reaction mixture was then transferred into a separating funnel and quickly washed successively with semi-saturated aqueous ammonium chloride solution and water. After drying over anhydrous magnesium sulphate, the mixture was filtered and the filtrate was freed from the solvent on a rotary evaporator. This gave 1.5 g (99% of theory) of the title compound.

MS (DCI, NH₃): m/z=276 [M+NH₄]⁺.

### Example 3A

### 3-(1-{[(Triisopropylsilyl)oxy]methyl}cyclopropyl)benzyl methanesulphonate

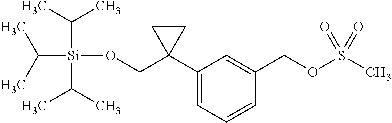 [SMILES: CC(C)[Si](OCC1(c2cccc(COS(C)(=O)=O)c2)CC1)(C(C)C)C(C)C]

### Step 1: Methyl 1-(3-bromophenyl)cyclopropanecarboxylate

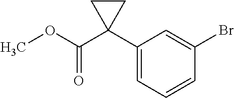

At 0° C., 48 ml (48.0 mmol) of a 1 M solution of lithium hexamethyldisilazide (LiHMDS) in THF were added to a solution of 10.0 g (43.6 mmol) of methyl (3-bromophenyl)acetate in 250 ml of anhydrous THF. After 15 min at 0° C., 4.9 ml (56.7 mmol) of 1,2-dibromoethane were added. The ice/water bath was removed, and the mixture was stirred at RT for another 1 h. The mixture was then cooled once more to 0° C., and a further 48 ml (48.0 mmol) of the LiHMDS solution were added. After the addition had ended, stirring was continued at RT for 63 h. About 250 ml of saturated aqueous ammonium chloride solution were then added, and the reaction mixture was extracted three times with about 200 ml of ethyl acetate each time. The combined organic extracts were washed successively with water and saturated sodium chloride solution, dried over anhydrous magnesium sulphate, filtered and finally freed from the solvent under reduced pressure. The residue obtained was purified by means of filtration with suction through silica gel using 20:1 cyclohexane/ethyl acetate as mobile phase. This gave 6.24 g (56% of theory) of the title compound.

¹H NMR (400 MHz, CDCl₃, δ/ppm): 7.50 (m, 1H), 7.39 (m, 1H), 7.27 (m, 1H), 7.19 (m, 1H), 3.63 (s, 3H), 1.62-1.60 (m, 2H), 1.20-1.17 (m, 2H).

GC/MS (Method 9, EIpos): Rₜ=5.27 min, m/z=254/256 [M]⁺.

### Step 2: [1-(3-Bromophenyl)cyclopropyl]methanol

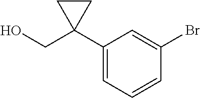 [SMILES: OCC1(c2cccc(Br)c2)CC1]

At −78° C., 13.7 ml (13.7 mmol) of a 1 M solution of lithium aluminium hydride in THF were added to a solution of 3.50 g (13.7 mmol) of the compound from Example 3A/step 1 in 70 ml of anhydrous THF. After 1 h of stirring, about 3 ml of saturated aqueous ammonium chloride solution were added and the reaction mixture was allowed to warm to RT. The mixture was then diluted with about 80 ml of ethyl acetate, and anhydrous magnesium sulphate was then added in such an amount that the aqueous phase was taken up completely. After filtration, the filtrate was concentrated and the residue was purified by means of MPLC (silica gel, mobile phase: cyclohexane→cyclohexane/ethyl acetate 5:1). This gave 1.37 g (44% of theory) of the title compound.

¹H NMR (400 MHz, CDCl₃, δ/ppm): 7.52 (s, 1H), 7.36 (d, 1H), 7.29 (d, 1H), 7.18 (t, 1H), 3.66 (d, 2H), 1.44 (t, 1H), 0.91-0.84 (m, 4H).

GC/MS (Method 9, EIpos): Rₜ=5.26 min, m/z=226/228 [M]⁺.

### Step 3: {[1-(3-Bromophenyl)cyclopropyl]methoxy}(triisopropyl)silane

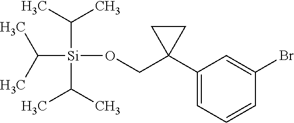 [SMILES: CC(C)[Si](OCC1(c2cccc(Br)c2)CC1)(C(C)C)C(C)C]

At about −50° C., 1.55 ml (6.19 mmol) of triisopropylsilyl triflate were added to a solution of 1.34 g (5.90 mmol) of the compound from Example 3A/step 2 and 948 mg (8.85 mmol) of 2,6-lutidine in 25 ml of anhydrous dichloromethane. After 30 min, the cooling bath was removed, and stirring was continued at RT for 1 h. Approx. 50 ml of water were then added and the mixture was extracted three times with about 50 ml of ethyl acetate each time. The combined organic extracts were washed with saturated sodium chloride solution, dried over anhydrous magnesium sulphate, filtered and finally freed from the solvent under reduced pressure. The residue obtained was purified by means of MPLC (silica gel, mobile phase: cyclohexane/ethyl acetate 5:1). This gave 1.93 g (85% of theory) of the title compound.

¹H NMR (400 MHz, CDCl₃, δ/ppm): 7.52 (s, 1H), 7.31 (d, 1H), 7.27 (d, 1H), 7.13 (t, 1H), 3.74 (s, 2H), 1.02 (m, 3H), 0.99 (d, 18H), 0.91-0.89 (m, 2H), 0.78-0.75 (m, 2H).

GC/MS (Method 9, EIpos): Rₜ=6.87 min, m/z=339/341 [M-ⁱPr]⁺.

### Step 4: 3-(1-{[(Triisopropylsilyl)oxy]methyl}cyclopropyl)benzaldehyde

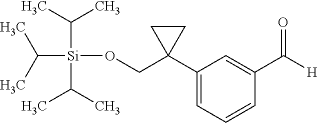 [SMILES: CC(C)[Si](OCC1(c2cccc(C=O)c2)CC1)(C(C)C)C(C)C]

Analogously to the process described under Example 2A/step 2, 1.92 g (5.01 mmol) of the compound from Example 3A/step 3 and corresponding amounts of n-butyllithium and N,N-dimethylformamide gave 1.48 g (88% of theory) of the title compound after MPLC purification (silica gel, mobile phase: cyclohexane/ethyl acetate 10:1).

¹H NMR (400 MHz, CDCl₃, δ/ppm): 10.00 (s, 1H), 7.89 (s, 1H), 7.72 (d, 1H), 7.65 (d, 1H), 7.43 (t, 1H), 3.79 (s, 2H), 1.01 (sept, 3H), 0.98 (d, 18H), 0.96-0.94 (m, 2H), 0.83-0.81 (m, 2H).

GC/MS (Method 9, EIpos): Rₜ=7.00 min, m/z=289 [M-ⁱPr]⁺.

### Step 5: [3-(1-{[(Triisopropylsilyl)oxy]methyl}cyclopropyl)phenyl]methanol

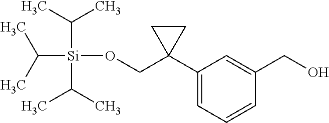 [SMILES: CC(C)[Si](OCC1(c2cccc(CO)c2)CC1)(C(C)C)C(C)C]

Analogously to the process described under Example 3A/step 2, 1.40 g (4.21 mmol) of the compound from Example 3A/step 4 and the corresponding amount of lithium aluminium hydride gave 1.10 g (78% of theory) of the title compound. Here, chromatographic purification of the product was dispensed with.

¹H NMR (400 MHz, CDCl₃, δ/ppm): 7.38 (s, 1H), 7.31-7.25 (m, 2H), 7.20 (d, 1H), 4.67 (d, 2H), 3.79 (s, 2H), 1.60 (t, 1H), 1.02 (sept, 3H), 1.00 (d, 18H), 0.93-0.90 (m, 2H), 0.77-0.75 (m, 2H).

GC/MS (Method 9, EIpos): Rₜ=7.18 min, m/z=291 [M-ⁱPr]⁺.

### Step 6: 3-(1-{[(Triisopropylsilyl)oxy]methyl}cyclopropyl)benzyl methanesulphonate

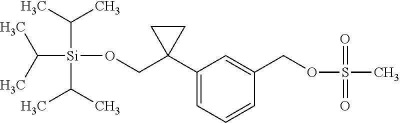 [SMILES: CC(C)[Si](OCC1(c2cccc(COS(C)(=O)=O)c2)CC1)(C(C)C)C(C)C]

Analogously to the process described under Example 2A/step 4, 820 mg (2.45 mmol) of the compound from Example 3A/step 5 and corresponding amounts of triethylamine and methanesulphonic anhydride gave 1.01 g (100% of theory) of the title compound.

¹H NMR (400 MHz, CDCl₃, δ/ppm): 7.42 (s, 1H), 7.40 (d, 1H), 7.32 (t, 1H), 7.25 (d, 1H), 5.21 (s, 2H), 3.77 (s, 2H), 2.91 (s, 3H), 1.02 (sept, 3H), 0.98 (d, 18H), 0.93-0.91 (m, 2H), 0.79-0.76 (m, 2H).

LC/MS (Method 3, ESIpos): Rₜ=1.59 min; m/z=413 [M+H]⁺.

MS (DCI, NH₃): m/z=430 [M+NH₄]⁺.

### Example 4A

### (2-Chloropyridin-4-yl)methyl methanesulphonate

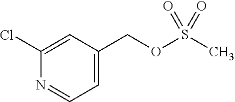

At 0° C., 12.9 ml (167 mmol) of methanesulphonyl chloride were added dropwise to a solution of 20 g (139 mmol) of (2-chloropyridin-4-yl)methanol and 25.2 ml (181 mmol) of triethylamine in 400 ml of THF, with the reaction temperature increasing to 20° C. during the addition. After removal of the cooling bath, the mixture was stirred at RT for another 30 min. A little water, saturated sodium bicarbonate solution and ethyl acetate were then added to the reaction mixture, and the phases were separated. The aqueous phase was reextracted once with ethyl acetate, and the combined organic phases were washed once with saturated sodium chloride solution, dried over magnesium sulphate and concentrated. The residue was triturated with pentane. The solid formed was filtered off, washed with pentane and dried under high vacuum. This gave 30.60 g (98% of theory) of the title compound.

¹H NMR (400 MHz, CDCl₃, δ/ppm): 8.44 (d, 1H), 7.37 (s, 1H), 7.25 (d, 1H), 5.22 (s, 2H), 3.10 (s, 3H).

LC/MS (Method 3, ESIpos): Rₜ=0.61 min; m/z=222 [M+H]⁺.

### Example 5A

### 1-[4-(Chloromethyl)pyridin-2-yl]-4-cyclopropylpiperazine

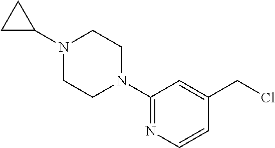 [SMILES: ClCc1ccnc(N2CCN(C3CC3)CC2)c1]

### Step 1: [2-(piperazin-1-yl)pyridin-4-yl]methanol

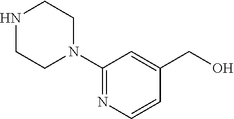 [SMILES: OCc1ccnc(N2CCNCC2)c1]

Under argon, 120 g (1.39 mol) of piperazine were added to 10.0 g (69.6 mmol) of (2-chloropyridin-4-yl)methanol. The mixture was heated at 150° C. overnight, while stirring. After cooling to RT, part of the excess piperazine which had deposited in the upper part of the reaction vessel was removed. The resinous contents of the flask were taken up in 700 ml of dichloromethane and the mixture was stirred at RT for 30 min. The solid that remained was filtered off, the filter cake was washed with dichloromethane and the solid was then discarded. The collected filtrate was concentrated and the residue was dried under reduced pressure. This gave 13.3 g (about 99% of theory) of the crude title compound which, according to ¹H NMR, still contained piperazine.

¹H NMR (400 MHz, CDCl₃, δ/ppm): 8.14 (d, 1H), 6.67 (s, 1H), 6.58 (d, 1H), 4.64 (s, 2H), 3.55-3.45 (m, 4H), 3.01-2.94 (m, 4H).

LC/MS (Method 4, ESIpos): Rₜ=0.19 min; m/z=194 [M+H]⁺.

### Step 2: [2-(4-Cyclopropylpiperazin-1-yl)pyridin-4-yl]methanol

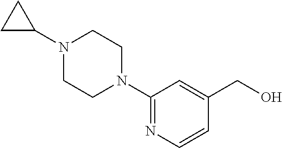 [SMILES: OCc1ccnc(N2CCN(C3CC3)CC2)c1]

13.1 g (67.9 mmol) of the compound from Example 5A/step 1 were dissolved in a mixture of 535 ml of methanol and 39 ml (679 mmol) of acetic acid. 9.2 g of molecular sieve (3 Å) and 82 ml (407 mmol) of [(1-ethoxycyclopropyl)oxy](trimethyl)silane were added. After stirring at RT for 10 min, 12.8 g (203 mmol) of sodium cyanoborohydride were added and the mixture was heated under reflux for 2 h, while stirring. After cooling to RT, the solid present was filtered off and rinsed twice with 20 ml of methanol each time. The filtrate was concentrated and the residue was taken up in 550 ml of dichloromethane. The mixture was washed twice with 500 ml of saturated aqueous sodium bicarbonate solution each time and once with 500 ml of saturated aqueous sodium chloride solution, dried over magnesium sulphate, filtered and concentrated. The residue was purified by means of column chromatography (silica gel, mobile phase: dichloromethane/methanol 95:5). Drying under reduced pressure gave 9.59 g (61% of theory) of the title compound.

¹H NMR (400 MHz, CDCl₃, δ/ppm): 8.13 (d, 1H), 6.67 (s, 1H), 6.57 (d, 1H), 4.63 (s, 2H), 3.58-3.46 (m, 4H), 2.77-2.66 (m, 4H), 1.70-1.60 (m, 1H), 0.55-0.41 (m, 4H).

LC/MS (Method 3, ESIpos): Rₜ=0.17 min; m/z=234 [M+H]⁺.

### Step 3: 1-[4-(Chloromethyl)pyridin-2-yl]-4-cyclopropylpiperazine

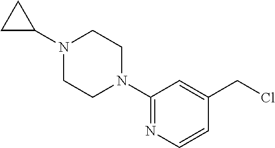 [SMILES: ClCc1ccnc(N2CCN(C3CC3)CC2)c1]

9.59 g (41.1 mmol) of the compound from Example 5A/step 2 were initially introduced into 60 ml of dichloromethane. 15 ml (205 mmol) of thionyl chloride were slowly added at RT and the mixture was stirred first at RT for 10 min, then under reflux for 4.5 h. After cooling to RT, 40 ml of water were added to the mixture and the mixture was rendered basic with 460 ml of saturated aqueous sodium bicarbonate solution and extracted three times with 500 ml of dichloromethane each time. The combined dichloromethane phases were dried over magnesium sulphate, filtered and concentrated. The residue was purified by means of column chromatography (silica gel, mobile phase: cyclohexane/ethyl acetate 7:3). Drying under reduced pressure gave 5.47 g (53% of theory) of the title compound.

¹H NMR (400 MHz, CDCl₃, δ/ppm): 8.16 (d, 1H), 6.68-6.56 (m, 2H), 4.45 (s, 2H), 3.61-3.45 (m, 4H), 2.79-2.67 (m, 4H), 1.69-1.62 (m, 1H), 0.58-0.35 (m, 4H).

LC/MS (Method 3, ESIpos): Rₜ=0.43 min, m/z=252/254 [M+H]⁺.

### Example 6A

### 1-(4-Methylbenzyl)-6-oxo-1,6-dihydropyridine-3-carboxylic acid

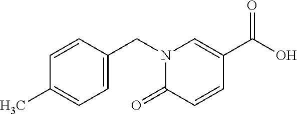 [SMILES: Cc1ccc(Cn2cc(C(=O)O)ccc2=O)cc1]

A solution of 2.0 g (14.4 mmol) of 6-oxo-1,6-dihydropyridine-3-carboxylic acid and 2.82 g (50.3 mmol) of potassium hydroxide in a mixture of 20 ml of methanol and 4 ml of water was heated at the boil for 5 min, and 5.32 g (28.7 mmol) of 1-(bromomethyl)-4-methylbenzene were then added at boiling point. The reaction mixture was then stirred at reflux for a further 1.5 h. After cooling to RT, the solvent was removed on a rotary evaporator and the residue was taken up in 250 ml of water. The aqueous phase was extracted twice with in each case 150 ml of tert-butyl methyl ether and then acidified with 20 ml of 1 M hydrochloric acid. The precipitate formed was filtered off, washed with water and dried under high vacuum. This gave 2.66 g of a crude product (purity 86%), 300 mg of which were used directly for preparing the compound in Example 1 (see there). The remaining 2.30 g of the crude product were purified by preparative HPLC (Method 10). This purification gave 1.54 g (22% of theory) of the title compound.

¹H-NMR (400 MHz, DMSO-d₆, δ/ppm): 12.87 (br. s, 1H), 8.53 (d, 1H), 7.78 (dd, 1H), 7.22 (d, 2H), 7.15 (d, 2H), 6.45 (d, 1H), 5.14 (s, 2H), 2.27 (s, 3H).

LC/MS (Method 3, ESIpos): Rₜ=0.74 min; m/z=244 [M+H]⁺.

### Example 7A

N′-Hydroxy-4-[(trifluoromethyl)sulphanyl]benzenecarboximidamide

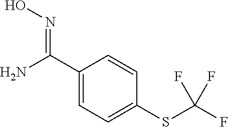 [SMILES: N/C(=N\O)c1ccc(SC(F)(F)F)cc1]

73 g (1.05 mol) of hydroxylammonium chloride were added to a solution of 113 g (500 mmol) of 4-[(trifluoromethyl)sulphanyl]benzenecarbonitrile and 147 ml (1.05 mol) of triethylamine in 1.4 litres of ethanol, and then the mixture was heated under reflux for 30 min. After cooling to RT, 1 litre of water was added and the mixture was extracted three times with a total of 1.4 litres of ethyl acetate. The combined organic extracts were washed successively with water and saturated sodium chloride solution. After drying over anhydrous magnesium sulphate, the mixture was filtered and the solvent was removed on a rotary evaporator. The resulting residue was taken up in 1 litre of cyclohexane, 40 ml of diisopropyl ether were added and the mixture was stirred at RT for 20 min. The resulting solid was filtered off with suction, and, after drying under high vacuum, a first portion of 78.6 g of the title compound was obtained. The filtrate was concentrated and the residue was subsequently stirred at boiling in a mixture of 100 ml of cyclohexane and 5 ml of ethyl acetate for 30 min. After cooling, the solid was filtered off with suction and dried under high vacuum. This gave a second portion of 4.2 g of the title compound. A total of 82.8 g (70% of theory) of the title compound was thus obtained.

¹H-NMR (400 MHz, DMSO-d₆, δ/ppm): 9.90 (s, 1H), 7.80 (d, 2H), 7.72 (d, 2H), 5.94 (s, 2H).

LC/MS (Method 4, ESIpos): Rₜ=1.42 min; m/z=237 [M+H]⁺.

### Example 8A

N′-Hydroxy-4-(1,1,1-trifluoro-2-methylpropan-2-yl)benzenecarboximidamide

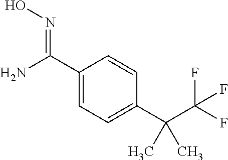 [SMILES: CC(C)(c1ccc(/C(N)=N/O)cc1)C(F)(F)F]

### Step 1: 2-(4-Bromophenyl)-1,1,1-trifluoropropan-2-ol

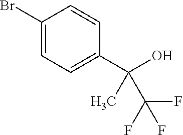

A suspension of dichloro(dimethyl)titanium in a heptane/dichloromethane mixture was first prepared as follows: 100 ml (100 mmol) of a 1 M solution of titanium tetrachloride in dichloromethane were cooled to −30° C., 100 ml (100 mmol) of a 1 M solution of dimethylzinc in heptane were added dropwise and the mixture was subsequently stirred at −30° C. for 30 min. This suspension was then cooled to −40° C. and a solution of 10 g (39.5 mmol) of 1-(4-bromophenyl)-2,2,2-trifluoroethanone in 50 ml of dichloromethane was added. The mixture was subsequently stirred at −40° C. for 5 min, the temperature was then allowed to come to RT and the mixture was stirred at RT for a further 2 h. 50 ml of water were slowly added dropwise, while cooling with ice, and the mixture was then diluted with a further 300 ml of water. It was extracted twice with dichloromethane, the combined dichloromethane phases were washed once with water, dried over anhydrous magnesium sulphate and filtered and the solvent was removed on a rotary evaporator. The residue was purified by column chromatography over silica gel (mobile phase: cyclohexane/ethyl acetate 85:15). 10.5 g (100% of theory) of the title compound were obtained, which, according to ¹H NMR, still contained residues of solvent.

¹H NMR (400 MHz, CDCl₃, δ/ppm): 7.52 (d, 2H), 7.47 (d, 2H), 1.76 (s, 3H).

LC/MS (Method 5, ESIpos): Rₜ=2.27 min; m/z=268 [M+H]⁺.

### Step 2: 2-(4-Bromophenyl)-1,1,1-trifluoropropan-2-yl methanesulphonate

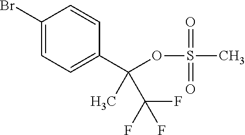 [SMILES: CC(OS(C)(=O)=O)(c1ccc(Br)cc1)C(F)(F)F]

3.12 g (78.05 mmol, 60% strength in mineral oil) of sodium hydride were initially introduced into 45 ml of THF under argon and a solution of 10.5 g (39.03 mmol) of the compound obtained in Example 8A/step 1 in 20 ml of THF was added dropwise at RT. After the mixture had been stirred at RT for 1 h and at 40° C. for 30 min, a solution of 8.94 g (78.05 mmol) of methanesulphonyl chloride in 45 ml of THF was added dropwise and the reaction mixture was stirred at 40° C. for a further 60 min. 50 ml of water were then slowly added dropwise to the mixture and the mixture was diluted with saturated aqueous sodium bicarbonate solution and extracted twice with ethyl acetate. The combined ethyl acetate phases were dried over anhydrous magnesium sulphate and filtered and the solvent was removed on a rotary evaporator. The residue was stirred in hexane and the solid obtained was filtered off and dried under reduced pressure. This gave 12.4 g (92% of theory) of the title compound.

¹H NMR (400 MHz, CDCl₃, δ/ppm): 7.58 (d, 2H), 7.43 (d, 2H), 3.16 (s, 3H), 2.28 (s, 3H).

LC/MS (Method 4, ESIpos): Rₜ=2.32 min, m/z=364 [M+NH₄]⁺.

### Step 3: 1-Bromo-4-(1,1,1-trifluoro-2-methylpropan-2-yl)benzene

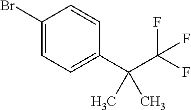

12.4 g (35.72 mmol) of the compound obtained in Example 8A/step 2 were initially introduced into 250 ml of dichloromethane and the mixture was cooled to 0° C. 35.7 ml (71.44 mmol) of a 2 M solution of trimethylaluminium were then slowly added dropwise at 0° C., while stirring, and the mixture was then allowed to come to RT and was subsequently stirred at RT for a further 1.5 h. 120 ml of saturated aqueous sodium hydrogencarbonate solution were slowly added dropwise to the mixture, followed by 40 ml of saturated aqueous sodium chloride solution. The mixture was filtered through kieselguhr and the kieselguhr was washed again twice with dichloromethane. The combined dichloromethane phases were washed once with saturated aqueous sodium chloride solution and dried over anhydrous magnesium sulphate, and the solvent was removed on a rotary evaporator. This gave 8.69 g (87% of theory) of the title compound in a purity of 95%.

¹H NMR (400 MHz, CDCl₃, δ/ppm): 7.49 (d, 2H), 7.33 (d, 2H), 1.55 (s, 6H).

GC/MS (Method 9, EIpos): Rₜ=3.48 min, m/z=266 [M]⁺.

### Step 4: 4-(1,1,1-Trifluoro-2-methylpropan-2-yl)benzenecarbonitrile

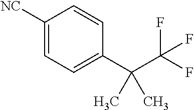

3.34 g (12.50 mmol) of the compound obtained in Example 8A/step 3 were initially introduced into 2.5 ml of degassed DMF under argon, 881 mg (7.50 mmol) of zinc cyanide and 867 mg (0.75 mmol) of tetrakis(triphenylphosphine)palladium(0) were added and the mixture was stirred at 80° C. overnight. After cooling to RT, the reaction mixture was diluted with ethyl acetate and solid constituents were filtered off. The filtrate was washed twice with 2 N aqueous ammonia solution and once with saturated aqueous sodium chloride solution, dried over anhydrous magnesium sulphate and freed from the solvent on a rotary evaporator. The residue was purified by column chromatography over silica gel (mobile phase: cyclohexane/ethyl acetate 85:15). This gave 2.08 g (78% of theory) of the title compound.

¹H NMR (400 MHz, CDCl₃, δ/ppm): 7.68 (d, 2H), 7.62 (d, 2H), 1.60 (s, 6H).

GC/MS (Method 9, EIpos): Rₜ=3.83 min, m/z=213 [M]⁺.

### Step 5: N′-Hydroxy-4-(1,1,1-trifluoro-2-methylpropan-2-yl)benzenecarboximidamide

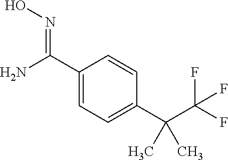 [SMILES: CC(C)(c1ccc(/C(N)=N/O)cc1)C(F)(F)F]

A mixture of 2.40 g (11.26 mmol) of the compound from Example 8A/step 4, 1.72 g (24.77 mmol) of hydroxylamine hydrochloride and 3.45 ml (24.77 mmol) of triethylamine in 60 ml of ethanol was stirred under reflux for 1 h. After cooling to RT, the solvent was removed on a rotary evaporator. Ethyl acetate was added to the residue and the solid present was filtered off. The ethyl acetate solution was washed successively with water and saturated aqueous sodium chloride solution, dried over anhydrous magnesium sulphate and filtered. After removal of the solvent, the oil obtained was triturated with petroleum ether. After the resulting solid had been filtered off with suction and dried under a high vacuum, 2.65 g (96% of theory) of the title compound were obtained.

¹H NMR (400 MHz, CDCl₃, δ/ppm): 8.0 (s, broad, 1H), 7.62 (d, 2H), 7.52 (d, 2H), 4.88 (s, broad, 2H), 1.60 (s, 6H).

LC/MS (Method 4, ESIpos): Rₜ=1.34 min; m/z=247 [M+H]⁺.

### Example 9A

### N-Hydroxy-4-[1-(trifluoromethyl)cyclopropyl]benzenecarboximidamide

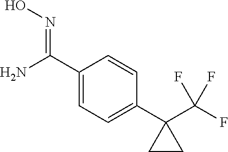 [SMILES: N/C(=N\O)c1ccc(C2(C(F)(F)F)CC2)cc1]

### Step 1: 1-Bromo-4-[1-(trifluoromethyl)cyclopropyl]benzene

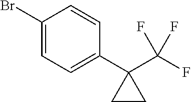 [SMILES: FC(F)(F)C1(c2ccc(Br)cc2)CC1]

Activated zinc bromide on montmorillonite was first prepared as follows: 7.0 g (31.1 mmol) of zinc bromide were initially charged in a 1 litre flask in 225 ml of methanol, and 28.2 g of K10 montmorillonite were added. Subsequently, the suspension was stirred at RT for 1 h. Then the mixture was concentrated to dryness on a rotary evaporator. The remaining fine powder was heated to bath temperature 200° C. in a sand bath under gentle vacuum (approx. 500 mbar) for 1 h and then allowed to cool under argon.

The title compound was then prepared as follows: 49.63 g (267 mmol) of 1-phenyl-1-(trifluoromethyl)cyclopropane were initially charged in 1.25 litres of pentane, and the activated zinc bromide on montmorillonite obtained above was added. Then the reaction vessel was wrapped with aluminium foil on the outside, in order to reduce the incidence of light. 137 ml (2.67 mol) of bromine were slowly added dropwise while stirring. Subsequently, the reaction mixture was stirred in the dark at RT for 16 h. Then, while cooling with ice, 1 litre of saturated aqueous sodium sulphite solution was added dropwise. The solids were filtered off with suction and washed twice with pentane. After phase separation, the filtrate was extracted twice more with 1 litre each time of pentane. The combined organic extracts were dried over anhydrous sodium sulphate, filtered and freed from the solvent on a rotary evaporator under only gentle vacuum. This gave 77.18 g (92% pure, 100% of theory) of the title compound.

¹H NMR (400 MHz, CDCl₃, δ/ppm): 7.47 (d, 2H), 7.33 (s, 2H), 1.37-1.34 (m, 2H), 1.03-0.98 (m, 2H).

GC/MS (Method 9, ESIpos): Rₜ=3.43 min, m/z=264/266 [M]⁺.

### Step 2: 4-[1-(Trifluoromethyl)cyclopropyl]benzonitrile

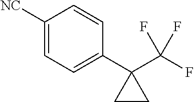 [SMILES: N#Cc1ccc(C2(C(F)(F)F)CC2)cc1]

A solution of 75.0 g (283 mmol) of the compound from Example 9A/Step 1 in a mixture of 990 ml of DMF and 10 ml of water was freed of oxygen by repeated application of a gentle vacuum and venting with argon. Then 37.87 g (322 mmol) of zinc cyanide and 32.69 g (28.3 mmol) of tetrakis(triphenylphosphine)palladium(0) were added. The reaction mixture was subsequently heated to 120° C. for 5 h. After cooling to RT, insolubles were filtered off and the residue was washed with a little DMF. The filtrate was subsequently freed from the solvent on a rotary evaporator. The resulting crude product was dissolved in 1.5 litres of ethyl acetate and the mixture was washed twice with 500 ml each time of saturated ammonium chloride solution and once with 500 ml of saturated sodium chloride solution. After drying the organic phase over anhydrous magnesium sulphate, the mixture was filtered and the filtrate was concentrated on a rotary evaporator. The oil obtained was purified by means of suction filtration through 175 g of silica gel with 40:1 cyclohexane/ethyl acetate as the eluent. This gave, after concentration of the product fractions and drying under high vacuum, 49.7 g (83% of theory) of the title compound.

¹H NMR (400 MHz, CDCl₃, δ/ppm): 7.65 (d, 2H), 7.57 (d, 2H), 1.46-1.42 (m, 2H), 1.09-1.03 (m, 2H).

GC/MS (Method 9, ESIpos): Rₜ=3.79 min, m/z=211 [M]⁺.

### Step 3: N′-Hydroxy-4-[1-(trifluoromethyl)cyclopropyl]benzenecarboximidamide

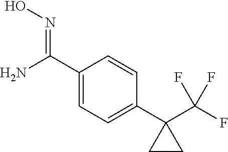 [SMILES: N/C(=N\O)c1ccc(C2(C(F)(F)F)CC2)cc1]

14.48 g (208 mmol) of hydroxylammonium chloride and 29 ml (208 mmol) of triethylamine were added to a solution of 20.0 g (94.7 mmol) of the compound from Example 9A/Step 2 in 500 ml of ethanol. The reaction mixture was heated under reflux for 2 h. Subsequently, about half of the solvent was removed on a rotary evaporator. 1.5 litres of water were added to the remaining mixture, and the resulting suspension was stirred at RT for 20 min. Then the solid was filtered off with suction, washed with a little cold water and dried under high vacuum. For further purification, it was stirred with a mixture of 120 ml of pentane and 30 ml of dichloromethane. This gave, after the solid had again been filtered off with suction and dried, 15.79 g (68% of theory) of the title compound.

¹H-NMR (400 MHz, DMSO-d₆, δ/ppm): 9.68 (s, 1H), 7.67 (d, 2H), 7.46 (d, 2H), 5.83 (s, broad, 2H), 1.36-1.32 (m, 2H), 1.15-1.11 (m, 2H).

LC/MS (Method 1, ESIpos): Rₜ=0.80 min; m/z=245 [M+H]⁺.

### Example 10A

### 5-{3-[4-(Trifluoromethoxy)phenyl]-1,2,4-oxadiazol-5-yl}pyridin-2(1H)-one

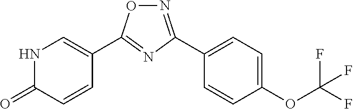 [SMILES: O=c1ccc(-c2nc(-c3ccc(OC(F)(F)F)cc3)no2)c[nH]1]

459 mg (3.00 mmol) of 1-hydroxy-1H-benzotriazole hydrate (HOBt) and 575 mg (3.00 mmol) of 1-(3-dimethylaminopropyl)-3-ethylcarbodiimide hydrochloride (EDC) were added to a solution of 417 mg (3.00 mmol) of 6-oxo-1,6-dihydropyridin-3-carboxylic acid (6-hydroxynicotinic acid) in 15 ml of DMF. The mixture was stirred at RT for 30 min. 660 mg (3.00 mmol) of N′-hydroxy-4-(trifluoromethoxy)benzenecarboximidamide were then added, and the mixture was stirred initially at RT for a further 30 min and then at 150° C. for 1 h. After cooling to RT, the reaction mixture was slowly introduced into 100 ml of water. The precipitate formed was filtered off and dried under vacuum. This gave 680 mg (69% of theory, purity 98%) of the title compound.

¹H-NMR (400 MHz, DMSO-d₆, δ/ppm): 12.48 (br. s, 1H), 8.38 (d, 1H), 8.21-8.16 (m, 2H), 8.04 (dd, 1H), 7.60 (d, 2H), 6.55 (d, 1H).

LC/MS (Method 3, ESIpos): Rₜ=0.95 min; m/z=324 [M+H]⁺.

### Example 11A

### 5-(3-{4-[(Trifluoromethyl)sulphanyl]phenyl}-1,2,4-oxadiazol-5-yl)pyridin-2(1H)-one

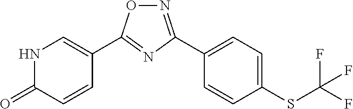 [SMILES: O=c1ccc(-c2nc(-c3ccc(SC(F)(F)F)cc3)no2)c[nH]1]

Analogously to the process described under Example 10A, 1.46 g (10.5 mmol) of 6-oxo-1,6-dihydropyridine-3-carboxylic acid and 2.48 g (10.5 mmol) of the compound from Example 7A gave 2.12 g (59% of theory) of the title compound.

¹H-NMR (400 MHz, DMSO-d₆, δ/ppm): 12.49 (br. s, 1H), 8.39 (d, 1H), 8.20 (d, 2H), 8.05 (dd, 1H), 7.94 (d, 2H), 6.55 (d, 1H).

LC/MS (Method 7, ESIpos): Rₜ=1.06 min; m/z=340 [M+H]⁺.

### Example 12A

### 5-{3-[4-(1,1,1-Trifluoro-2-methylpropan-2-yl)phenyl]-1,2,4-oxadiazol-5-yl}pyridin-2(1H)-one

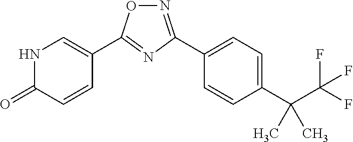 [SMILES: CC(C)(c1ccc(-c2noc(-c3ccc(=O)[nH]c3)n2)cc1)C(F)(F)F]

Analogously to the process described under Example 10A, 3.41 g (24.5 mmol) of 6-oxo-1,6-dihydropyridine-3-carboxylic acid and 6.04 g (24.5 mmol) of the compound from Example 8A gave 5.23 g (58% of theory, purity 94%) of the title compound.

¹H NMR (400 MHz, DMSO-d₆, δ/ppm): 12.47 (br. s, 1H), 8.37 (d, 1H), 8.08 (d, 2H), 8.04 (dd, 1H), 7.77 (d, 1H), 6.55 (d, 2H), 1.61 (s, 6H).

LC/MS (Method 1, ESIpos): Rₜ=1.26 min; m/z=350 [M+H]⁺.

### Example 13A

### 5-(3-{4-[1-(Trifluoromethyl)cyclopropyl]phenyl}-1,2,4-oxadiazol-5-yl)pyridin-2(1H)-one

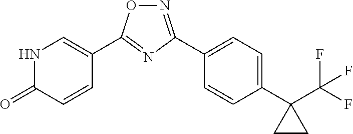 [SMILES: O=c1ccc(-c2nc(-c3ccc(C4(C(F)(F)F)CC4)cc3)no2)c[nH]1]

Analogously to the process described under Example 10A, 2.0 g (14.4 mmol) of 6-oxo-1,6-dihydropyridine-3-carboxylic acid and 3.51 g (14.4 mmol) of the compound from Example 9A gave 3.46 g (69% of theory) of the title compound.

¹H NMR (400 MHz, DMSO-d₆, δ/ppm): 12.47 (br. s, 1H), 8.37 (d, 1H), 8.07 (d, 2H), 8.04 (dd, 1H), 7.68 (d, 2H), 6.55 (d, 1H), 1.43-1.39 (m, 2H), 1.23-1.19 (m, 2H).

LC/MS (Method 3, ESIpos): Rₜ=1.04 min; m/z=348 [M+H]⁺.

### Example 14A

### 6-{3-[4-(Trifluoromethoxy)phenyl]-1,2,4-oxadiazol-5-yl}pyridazin-3(2H)-one

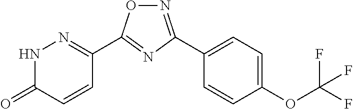 [SMILES: O=c1ccc(-c2nc(-c3ccc(OC(F)(F)F)cc3)no2)n[nH]1]

3 ml (21.4 mmol) of triethylamine, 1.64 g (10.7 mmol) of 1-hydroxy-1H-benzotriazole hydrate (HOBt) and 2.05 g (10.7 mmol) of 1-(3-dimethylaminopropyl)-3-ethylcarbodiimide hydrochloride (EDC) were added to a solution of 1.50 g (10.7 mmol) of 6-oxo-1,6-dihydropyridazine-3-carboxylic acid in 60 ml of DMF. The mixture was stirred at RT for 30 min. 2.75 g (10.7 mmol) of N′-hydroxy-4-(trifluoromethoxy)benzenecarboximidamide were then added, and the mixture was stirred initially at RT for 60 min and then at 150° C. for 1 h. After cooling to RT, the reaction mixture was slowly introduced into 500 ml of water. The precipitate formed was filtered off and dried under vacuum. This gave 1.88 g (53% of theory) of the title compound.

¹H NMR (400 MHz, DMSO-d₆, δ/ppm): 13.90 (br. s, 1H), 8.22 (d, 2H), 8.10 (d, 1H), 7.62 (d, 2H), 7.13 (d, 1H).

LC/MS (Method 7, ESIpos): Rₜ=1.00 min; m/z=325 [M+H]⁺.

### Example 15A

### 6-{3-[4-(1,1,1-Trifluoro-2-methylpropan-2-yl)phenyl]-1,2,4-oxadiazol-5-yl}pyridazin-3(2H)-one

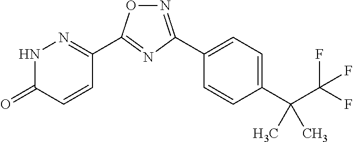 [SMILES: CC(C)(c1ccc(-c2noc(-c3ccc(=O)[nH]n3)n2)cc1)C(F)(F)F]

1.64 g (10.7 mmol) of 1-hydroxy-1H-benzotriazole hydrate (HOBt) and 2.05 g (10.7 mmol) of 1-(3-dimethylaminopropyl)-3-ethylcarbodiimide hydrochloride (EDC) were added to a solution of 1.50 g (10.7 mmol) of 6-oxo-1,6-dihydropyridazine-3-carboxylic acid in 60 ml of DMF. The mixture was stirred at RT for 30 min. 2.64 g (10.7 mmol) of the compound from Example 8A were then added, and the mixture was stirred initially at RT for a further 30 min and then at 150° C. for 1 h. After cooling to RT, the reaction mixture was slowly introduced into 500 ml of water. The precipitate formed was filtered off and dried under high vacuum. This gave 2.69 g (71% of theory) of the title compound.

¹H NMR (400 MHz, DMSO-d₆, δ/ppm): 13.89 (br. s, 1H), 8.11 (m, 3H), 7.79 (d, 2H), 7.13 (d, 1H), 1.62 (s, 6H).

LC/MS (Method 3, ESIpos): Rₜ=1.01 min; m/z=351 [M+H]⁺.

### Example 16A

### 3-{[tert-Butyl(diphenyl)silyl]oxy}azetidine

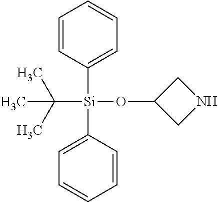 [SMILES: CC(C)(C)[Si](OC1CNC1)(c1ccccc1)c1ccccc1]

### Step 1: tert-Butyl 3-{[tert-butyl(diphenyl) silyl]oxy}azetidine-1-carboxylate

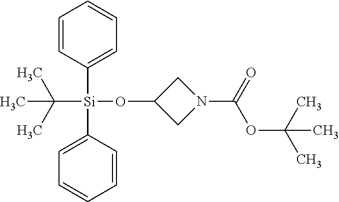 [SMILES: CC(C)(C)OC(=O)N1CC(O[Si](c2ccccc2)(c2ccccc2)C(C)(C)C)C1]

20.0 g (115 mmol) of tert-butyl 3-hydroxyazetidine-1-carboxylate and 9.43 g (139 mmol) of imidazole were initially charged in 200 ml of anhydrous DMF, and 34.91 g (127 mmol) of tent-butyl(diphenyl)silyl chloride were added at RT. After the reaction mixture had been stirred at RT for 18 h, it was poured into 3.2 litres of water and then extracted three times with approx. 1 litre each time of diethyl ether. The combined organic extracts were washed successively with saturated sodium hydrogencarbonate solution, water and saturated sodium chloride solution. After drying over anhydrous magnesium sulphate, the mixture was filtered and the solvent was removed on a rotary evaporator. The remaining residue was stirred with 100 ml of pentane for a few minutes.

Subsequently, the solid was filtered off with suction and dried under high vacuum. This gave 29.18 g (61% of theory) of the title compound.

¹H NMR (400 MHz, CDCl₃, δ/ppm): 7.60 (d, 4H), 7.46-7.37 (m, 6H), 4.53-4.49 (m, 1H), 3.93 (dd, 2H), 3.87 (dd, 2H), 1.41 (s, 9H), 1.04 (s, 9H).

LC/MS (Method 3, ESIpos): Rₜ=1.65 min; m/z=412 [M+H]⁺.

### Step 2: 3-{[tert-Butyl(diphenyl)silyl]oxy}azetidine

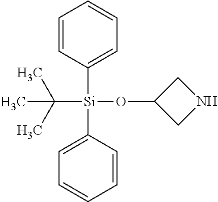 [SMILES: CC(C)(C)[Si](OC1CNC1)(c1ccccc1)c1ccccc1]

70 ml of trifluoroacetic acid (TFA) were added dropwise at RT to a solution of 20.0 g (48.6 mmol) of the compound from Example 16A/Step 1 in 70 ml of dichloromethane. After the reaction mixture had been stirred at RT for 30 min, all volatile components were removed on a rotary evaporator. 1 litre of 1 M sodium hydroxide solution was added to the remaining residue, which was extracted three times with approx. 200 ml each time of dichloromethane. The combined organic extracts were dried over anhydrous magnesium sulphate, filtered and finally concentrated to dryness on a rotary evaporator. After the residue had been dried under high vacuum, 14.85 g (98% of theory) of the title compound were obtained.

¹H NMR (400 MHz, CDCl₃, δ/ppm): 7.61 (d, 4H), 7.45-7.36 (m, 6H), 4.64-4.58 (m, 1H), 3.68 (dd, 2H), 3.53 (dd, 2H), 2.19 (broad, 1H), 1.03 (s, 9H).

LC/MS (Method 3, ESIpos): Rₜ=0.90 min; m/z=312 [M+H]⁺.

### Example 17A

### 1-(3-Bromobenzyl)-5-{3-[4-(trifluoromethoxy)phenyl]-1,2,4-oxadiazol-5-yl}pyridin-2(1H)-one

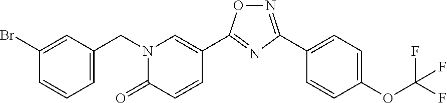 [SMILES: O=c1ccc(-c2nc(-c3ccc(OC(F)(F)F)cc3)no2)cn1Cc1cccc(Br)c1]

Under argon and with ice cooling, 80 mg (2.01 mmol, 60% in mineral oil) of sodium hydride were added to a mixture of 500 mg (1.55 mmol) of the compound from Example 10A in 10 ml of DMF. After 15 min of stirring at RT, 503 mg (2.01 mmol) of 3-bromobenzyl bromide were added, and the reaction mixture was stirred at RT for a further 30 min. Subsequently, 70 ml each of water and ethyl acetate were added slowly, and the phases were separated. The aqueous phase was extracted with 70 ml of ethyl acetate, and the combined organic phases were dried over sodium sulphate, filtered and concentrated. The residue was triturated with 15 ml of cyclohexane/ethyl acetate 4:1. The remaining solid was filtered off and washed with 4 ml of cyclohexane/ethyl acetate 4:1. Drying under high vacuum gave, as a first batch, 520 mg (68% of theory) of the title compound. The mother liquor obtained when filtering off was concentrated and the residue was purified by column chromatography (Biotage, silica gel, mobile phase cyclohexane/ethyl acetate 4:1). Drying under high vacuum thus gave a further 144 mg (19% of theory) of the title compound. In total, 664 mg (87% of theory) of the title compound were obtained in this manner.

¹H NMR (400 MHz, CDCl₃, δ/ppm): 8.35 (d, 1H), 8.16 (d, 2H), 8.03 (dd, 1H), 7.52 (s, 1H), 7.49 (d, 1H), 7.36-7.27 (m, 4H), 6.76 (d, 1H), 5.21 (s, 2H).

LC/MS (Method 3, ESIpos): Rₜ=1.34 min; m/z=492 [M+H]⁺.

### Example 18A

### 1-(3-Bromobenzyl)-5-(3-{4-[(trifluoromethyl)sulphanyl]phenyl}-1,2,4-oxadiazol-5-yl)pyridin-2(1H)-one

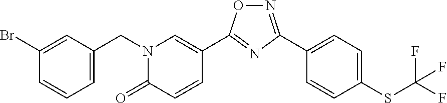 [SMILES: O=c1ccc(-c2nc(-c3ccc(SC(F)(F)F)cc3)no2)cn1Cc1cccc(Br)c1]

Under argon and at a temperature of 0° C., 153 mg (3.83 mmol) of sodium hydride (60% suspension in mineral oil) were added to a suspension of 1.0 g (2.95 mmol) of the compound from Example 11A in 20 ml of anhydrous DMF. After 15 min, 958 mg (3.83 mmol) of 3-bromobenzyl bromide were added, and cooling was removed. After the reaction mixture had been stirred at RT for about 16 h, 200 ml of water were added carefully and the mixture was extracted four times with about 50 ml of ethyl acetate each time. The combined organic extracts were washed with saturated sodium chloride solution, dried over anhydrous magnesium sulphate, filtered and freed from the solvent on a rotary evaporator. The residue that remained was chromatographed on silica gel using the mobile phase cyclohexane/ethyl acetate 2:1. This gave 1.20 g (80% of theory) of the title compound.

¹H NMR (400 MHz, DMSO-d₆, δ/ppm): 9.09 (d, 1H), 8.20 (d, 2H), 8.09 (dd, 1H), 7.95 (d, 2H), 7.63 (s, 1H), 7.52 (d, 1H), 7.40 (d, 1H), 7.33 (t, 1H), 6.66 (d, 1H), 5.27 (s, 2H).

LC/MS (Method 3, ESIpos): Rₜ=1.41 min, m/z=508/510 [M+H]⁺.

### Example 19A

### 1-(3-Bromobenzyl)-5-{3-[4-(1,1,1-trifluoro-2-methylpropan-2-yl)phenyl]-1,2,4-oxadiazol-5-yl}pyridin-2(1H)-one

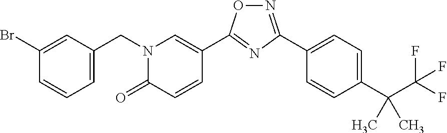 [SMILES: CC(C)(c1ccc(-c2noc(-c3ccc(=O)n(Cc4cccc(Br)c4)c3)n2)cc1)C(F)(F)F]

Analogously to the process described in Example 17A, 540 mg (1.55 mmol) of the compound from Example 12A and 503 mg (2.01 mmol) of 3-bromobenzyl bromide gave a total of 649 mg (81% of theory) of the title compound. Here, the chromatographic purification of the concentrated mother liquor was carried out using the mobile phase cyclohexane/ethyl acetate 7:3.

¹H NMR (400 MHz, DMSO-d₆, δ/ppm): 9.07 (d, 1H), 8.10-8.06 (m, 3H), 7.78 (d, 2H), 7.63 (s, 1H), 7.52 (d, 1H), 7.39 (d, 1H), 7.33 (t, 1H), 6.66 (d, 1H), 5.27 (s, 2H), 1.61 (s, 6H).

LC/MS (Method 3, ESIpos): Rₜ=1.39 min; m/z=518 [M+H]⁺.

### Example 20A

### 1-(3-Bromobenzyl)-5-(3-{4-[1-(trifluoromethyl)cyclopropyl]phenyl}-1,2,4-oxadiazol-5-yl)pyridin-2(1H)-one

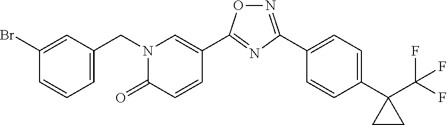 [SMILES: O=c1ccc(-c2nc(-c3ccc(C4(C(F)(F)F)CC4)cc3)no2)cn1Cc1cccc(Br)c1]

Under argon and at a temperature of 0° C., 150 mg (3.74 mmol) of sodium hydride (60% suspension in mineral oil) were added to a suspension of 1.0 g (2.88 mmol) of the compound from Example 13A in 20 ml of anhydrous DMF. After 15 min, 936 mg (3.74 mmol) of 3-bromobenzyl bromide were added, and cooling was removed. After the reaction mixture had been stirred at RT for about 16 h, 200 ml of water were added carefully and the mixture was extracted four times with about 50 ml of ethyl acetate each time. The combined organic extracts were washed with saturated sodium chloride solution, dried over anhydrous magnesium sulphate, filtered and freed from the solvent on a rotary evaporator. The residue that remained was stirred in a mixture of cyclohexane and ethyl acetate (2:1) at RT. The solid was filtered off with suction, washed with a small amount of the same solvent mixture and dried under high vacuum. Thus, a first fraction of 695 mg of the title compound was obtained. The concentrated mother liquor was chromatographed on silica gel using the mobile phase cyclohexane/ethyl acetate 2:1. A second batch of 544 mg of the title compound was obtained in this manner. Thus, the total yield was 1.24 g (83% of theory).

¹H NMR (400 MHz, DMSO-d₆, δ/ppm): 9.07 (d, 1H), 8.08 (d+dd, zus. 4H), 7.69 (d, 2H), 7.63 (s, 1H), 7.52 (d, 1H), 7.39 (d, 1H), 7.33 (t, 1H), 6.65 (d, 1H), 5.27 (s, 2H), 1.43-1.40 (m, 2H), 1.23-1.19 (m, 2H).

LC/MS (Method 2, ESIpos): Rₜ=2.86 min, m/z=516/518 [M+H]⁺.

### Example 21A

### 3-{[2-Oxo-5-{3-[4-(trifluoromethoxy)phenyl]-1,2,4-oxadiazol-5-yl}pyridin-1(2H)-yl]methyl}benzoic acid

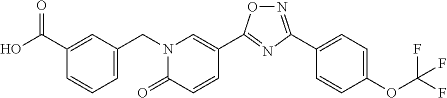 [SMILES: O=C(O)c1cccc(Cn2cc(-c3nc(-c4ccc(OC(F)(F)F)cc4)no3)ccc2=O)c1]

15.4 ml (15.4 mmol) of 1 M aqueous sodium hydroxide solution were added to a mixture of 1.25 g (2.65 mmol) of the compound from Example 18 in 60 ml of methanol, and the mixture was stirred under reflux for 30 min. After cooling to RT, the solvent was removed on a rotary evaporator and 23 ml of 1 M hydrochloric acid were added to the residue. After 15 min of stirring at RT, the solid formed was filtered off and washed twice with in each case 10 ml of water. Drying under high vacuum gave 1.14 g (94% of theory) of the title compound.

¹H NMR (400 MHz, DMSO-d₆, δ/ppm): 13.07 (br. s, 1H), 9.12 (d, 1H), 8.19 (d, 2H), 8.08 (dd, 1H), 7.97 (s, 1H), 7.88 (d, 1H), 7.66 (d, 1H), 7.60 (d, 2H), 7.50 (t, 1H), 6.66 (d, 1H), 5.35 (s, 2H).

LC/MS (Method 3, ESIpos): Rₜ=1.08 min; m/z=458 [M+H]⁺.

### Example 22A

### 3-{[2-Oxo-5-{3-[4-(trifluoromethoxy)phenyl]-1,2,4-oxadiazol-5-yl}pyridin-1(2H)-yl]methyl}benzoyl chloride

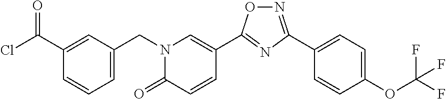 [SMILES: O=C(Cl)c1cccc(Cn2cc(-c3nc(-c4ccc(OC(F)(F)F)cc4)no3)ccc2=O)c1]

At RT, 0.55 ml (6.25 mmol) of oxalyl chloride and a few drops of DMF were added to a solution of 572 mg (1.25 mmol) of the compound from Example 21A in 20 ml of dichloromethane. After 2 h of stirring at RT, the solvent was removed on a rotary evaporator. The crude product obtained in this manner was used directly for further reactions.

### Example 23A

### 3-{[2-oxo-5-{3-[4-(1,1,1-trifluoro-2-methylpropan-2-yl)phenyl]-1,2,4-oxadiazol-5-yl}pyridin-1(2H)-yl]methyl}benzoic acid

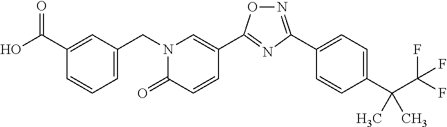 [SMILES: CC(C)(c1ccc(-c2noc(-c3ccc(=O)n(Cc4cccc(C(=O)O)c4)c3)n2)cc1)C(F)(F)F]

Analogously to the process described in Example 21A, 869 mg (1.75 mmol) of the compound from Example 19 gave 843 mg (100% of theory) of the title compound.

¹H-NMR (400 MHz, DMSO-d₆, δ/ppm): about 13.0 (very broad, 1H), 9.10 (d, 1H), 8.08 (d, 3H), 7.96 (s, 1H), 7.87 (d, 1H), 7.77 (d, 2H), 7.61 (d, 1H), 7.47 (t, 1H), 6.66 (d, 1H), 5.34 (s, 2H), 1.61 (s, 6H).

LC/MS (Method 3, ESIpos): Rₜ=1.17 min; m/z=484 [M+H]⁺.

### Example 24A

### 3-{[2-Oxo-5-{3-[4-(1,1,1-trifluoro-2-methylpropan-2-yl)phenyl]-1,2,4-oxadiazol-5-yl}pyridin-1(2H)-yl]methyl}benzoyl chloride

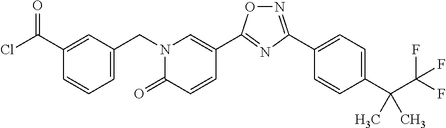 [SMILES: CC(C)(c1ccc(-c2noc(-c3ccc(=O)n(Cc4cccc(C(=O)Cl)c4)c3)n2)cc1)C(F)(F)F]

Analogously to the process described in Example 22A, 367 mg (0.760 mmol) of the compound from Example 23A were used to prepare the title compound as crude product which was used directly for further reactions.

### Example 25A

### 5-{3-[4-(Trifluoromethoxy)phenyl]-1,2,4-oxadiazol-5-yl}-1-[3-(1-{[(triisopropylsilyl)oxy]methyl}cyclopropyl)benzyl]pyridin-2(1H)-one

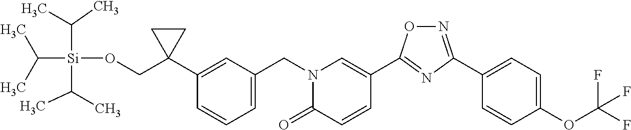 [SMILES: CC(C)[Si](OCC1(c2cccc(Cn3cc(-c4nc(-c5ccc(OC(F)(F)F)cc5)no4)ccc3=O)c2)CC1)(C(C)C)C(C)C]

Under argon and with ice cooling, 48 mg (1.21 mmol, 60% in mineral oil) of sodium hydride were added to a mixture of 300 mg (0.928 mmol) of the compound from Example 10A in 4 ml of DMF. After 15 min of stirring at RT, 421 mg (1.02 mmol) of the compound from Example 3A were added, and the reaction mixture was stirred at RT for another 2 h. Subsequently, 30 ml each of water and ethyl acetate were added slowly, and the phases were separated. The aqueous phase was extracted twice with in each case 30 ml of ethyl acetate, and the combined organic phases were washed with 50 ml of saturated sodium chloride solution, dried over sodium sulphate, filtered and concentrated. The residue was purified by column chromatography (Biotage, silica gel, mobile phase cyclohexane/ethyl acetate 9:1). Drying under high vacuum gave 366 mg (62% of theory) of the title compound.

¹H NMR (400 MHz, CDCl₃, δ/ppm): 8.32 (d, 1H), 8.14 (d, 2H), 8.01 (dd, 1H), 7.39-7.27 (m, 5H), 7.18 (d, 1H), 6.75 (d, 1H), 5.22 (s, 2H), 3.76 (s, 2H), 0.96-0.93 (m, 21H), 0.92-0.88 (m, 2H), 0.79-0.75 (m, 2H).

LC/MS (Method 3, ESIpos): Rₜ=1.87 min; m/z=640 [M+H]⁺.

### Example 26A

### 5-{3-[4-(1,1,1-Trifluoro-2-methylpropan-2-yl)phenyl]-1,2,4-oxadiazol-5-yl}-1-[3-(1-{[(triisopropylsilyl)oxy]methyl}cyclopropyl)benzyl]pyridin-2(1H)-one

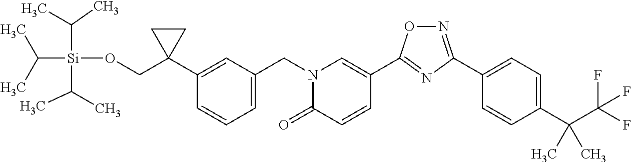 [SMILES: CC(C)[Si](OCC1(c2cccc(Cn3cc(-c4nc(-c5ccc(C(C)(C)C(F)(F)F)cc5)no4)ccc3=O)c2)CC1)(C(C)C)C(C)C]

Analogously to the process described in Example 25A, 162 mg (0.463 mmol) of the compound from Example 12A and 210 mg (0.509 mmol) of the compound from Example 3A gave 191 mg (61% of theory) of the title compound.

¹H NMR (400 MHz, CDCl₃, δ/ppm): 8.33 (d, 1H), 8.08 (d, 2H), 8.02 (dd, 1H), 7.62 (d, 2H), 7.39-7.33 (m, 2H), 7.31-7.27 (m, 1H), 7.18 (d, 1H), 6.74 (d, 1H), 5.22 (s, 2H), 3.76 (s, 2H), 1.62 (s, 6H), 1.02-0.92 (m, 21H), 0.92-0.88 (m, 2H), 0.79-0.75 (m, 2H).

LC/MS (Method 8, ESIpos): Rₜ=6.93 min; m/z=666 [M+H]⁺.

### Example 27A

### 1-[(2-Chloropyridin-4-yl)methyl]-5-{3-[4-(trifluoromethoxy)phenyl]-1,2,4-oxadiazol-5-yl}pyridin-2(1H)-one

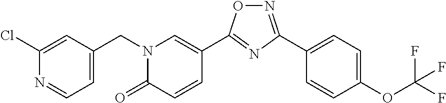 [SMILES: O=c1ccc(-c2nc(-c3ccc(OC(F)(F)F)cc3)no2)cn1Cc1ccnc(Cl)c1]

Under argon and with ice cooling, 485 mg (1.50 mmol) of the compound from Example 10A were added to a suspension of 78 mg (1.95 mmol, 60% in mineral oil) of sodium hydride in 9 ml DMF. After 15 min of stirring, 432 mg (1.95 mmol) of the compound from Example 4A were added, and the mixture was stirred at RT for another 16 h. 100 ml of water were then added slowly, and the mixture was extracted twice with in each case 70 ml of ethyl acetate. The combined organic phases were washed once with 100 ml of saturated sodium chloride solution, dried over sodium sulphate, filtered and concentrated. The residue was triturated with 10 ml of cyclohexane/ethyl acetate 7:3. The precipitate formed was washed with 4 ml of cyclohexane/ethyl acetate 4:1 and dried under high vacuum. This gave 342 mg (51% of theory) of the title compound.

¹H NMR (400 MHz, DMSO-d₆, δ/ppm): 9.08 (d, 1H), 8.38 (d, 1H), 8.20 (d, 2H), 8.12 (dd, 1H), 7.61 (d, 2H), 7.46 (s, 1H), 7.32 (d, 1H), 6.68 (d, 1H), 5.32 (s, 2H).

LC/MS (Method 3, ESIpos): Rₜ=1.20 min; m/z=449 [M+H]⁺.

### Example 28A

### 1-[(2-Chloropyridin-4-yl)methyl]-5-(3-{4-[(trifluoromethyl)sulphanyl]phenyl}-1,2,4-oxadiazol-5-yl)pyridin-2(1H)-one

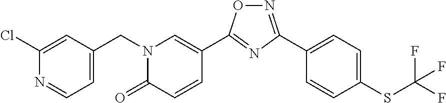 [SMILES: O=c1ccc(-c2nc(-c3ccc(SC(F)(F)F)cc3)no2)cn1Cc1ccnc(Cl)c1]

Analogously to the process described in Example 20A, 1.0 g (2.95 mmol) of the compound from Example 11A and 849 mg of the compound from Example 4A gave 874 mg (64% of theory) in total of the title compound. Here, the crude product initially obtained was triturated with pure ethyl acetate, resulting in a first fraction of the title compound of 647 mg. A second fraction of 227 mg was—as described in Example 20A—obtained by chromatography of the concentrated mother liquor.

¹H-NMR (400 MHz, DMSO-d₆, δ/ppm): 9.09 (s, 1H), 8.38 (d, 1H), 8.21 (d, 2H), 8.13 (d, 1H), 7.96 (d, 2H), 7.46 (s, 1H), 7.32 (d, 1H), 6.68 (d, 1H), 5.32 (s, 2H).

LC/MS (Method 3, ESIpos): Rₜ=1.24 min, m/z=465/467 [M+H]⁺.

### Example 29A

### 1-[(2-Chloropyridin-4-yl)methyl]-5-{3-[4-(1,1,1-trifluoro-2-methylpropan-2-yl)phenyl]-1,2,4-oxadiazol-5-yl}pyridin-2(1H)-one

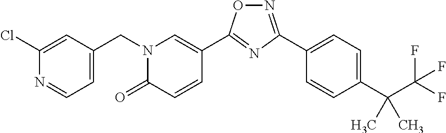 [SMILES: CC(C)(c1ccc(-c2noc(-c3ccc(=O)n(Cc4ccnc(Cl)c4)c3)n2)cc1)C(F)(F)F]

Under argon and with ice cooling, 540 mg (1.55 mmol) of the compound from Example 12A were added to a suspension of 80 mg (2.01 mmol, 60% in mineral oil) of sodium hydride in 10 ml of DMF. After 15 min of stirring at RT, 446 mg (2.01 mmol) of the compound from Example 4A were added, and the mixture was stirred at RT for another 30 min. Subsequently, 70 ml each of water and ethyl acetate were added slowly, and the phases were separated. The aqueous phase was extracted once more with 70 ml of ethyl acetate. The combined organic phases were dried over sodium sulphate, filtered and concentrated. The residue was purified by column chromatography (Biotage, silica gel, mobile phase cyclohexane/ethyl acetate 4:1). Drying under high vacuum gave 434 mg (56% of theory) of the title compound.

¹H-NMR (400 MHz, DMSO-d₆, δ/ppm): 9.07 (d, 1H), 8.38 (d, 1H), 8.13 (dd, 1H), 8.09 (d, 2H), 7.78 (d, 2H), 7.46 (s, 1H), 7.32 (dd, 1H), 6.68 (d, 1H), 5.32 (s, 2H), 1.61 (s, 6H).

LC/MS (Method 3, ESIpos): Rₜ=1.20 min; m/z=475 [M+H]⁺.

### Example 30A

### 1-[(2-Chloropyridin-4-yl)methyl]-5-(3-{4-[1-(trifluoromethyl)cyclopropyl]phenyl}-1,2,4-oxadiazol-5-yl)pyridin-2(1H)-one

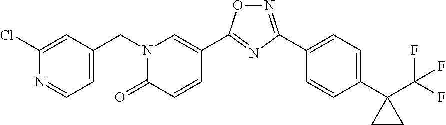 [SMILES: O=c1ccc(-c2nc(-c3ccc(C4(C(F)(F)F)CC4)cc3)no2)cn1Cc1ccnc(Cl)c1]

Analogously to the process described in Example 20A, 1.0 g (2.88 mmol) of the compound from Example 13A and 830 mg of the compound from Example 4A gave 1.0 g (74% of theory) of the title compound. Here, the crude product initially obtained was, after aqueous work-up, initially purified by MPLC (silica gel, mobile phase cyclohexane/ethyl acetate 1:1), followed by a second purification step by preparative HPLC (Method 16).

¹H-NMR (400 MHz, DMSO-d₆, δ/ppm): 9.07 (d, 1H), 8.38 (d, 1H), 8.12 (dd, 1H), 8.08 (d, 2H), 7.69 (d, 2H), 7.46 (s, 1H), 7.32 (dd, 1H), 6.67 (d, 1H), 5.32 (s, 2H), 1.43-1.40 (m, 2H), 1.23-1.19 (m, 2H).

LC/MS (Method 7, ESIpos): Rₜ=1.25 min, m/z=473/475 [M+H]⁺.

### Example 31A

### 1-{[2-(piperazin-1-yl)pyridin-4-yl]methyl}-5-{3-[4-(trifluoromethoxy)phenyl]-1,2,4-oxadiazol-5-yl}pyridin-2(1H)-one

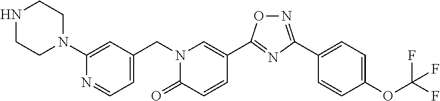 [SMILES: O=c1ccc(-c2nc(-c3ccc(OC(F)(F)F)cc3)no2)cn1Cc1ccnc(N2CCNCC2)c1]

A mixture of 168 mg (0.375 mmol) of the compound from Example 27A and 646 mg (7.50 mmol) of piperazine in 9 ml of ethanol was stirred in a microwave oven (Biotage Initiator, with dynamic irradiation power control) at 180° C. for 75 min. After cooling to RT, the solvent was removed on a rotary evaporator and 50 ml each of water and ethyl acetate were added to the residue. After phase separation, the aqueous phase was extracted three times with in each case 50 ml of ethyl acetate. The combined organic phases were dried over magnesium sulphate, filtered and concentrated. After drying under a high vacuum, 172 mg (69% of theory, purity 75%) of the title compound were obtained.

LC/MS (Method 3, ESIpos): Rₜ=0.81 min; m/z=499 [M+H]⁺.

### Example 32A

### 1-{[2-(piperazin-1-yl)pyridin-4-yl]methyl}-5-(3-{4-[(trifluoromethyl)sulphanyl]phenyl}-1,2,4-oxadiazol-5-yl)pyridin-2(1H)-one

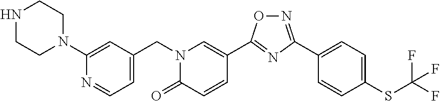 [SMILES: O=c1ccc(-c2nc(-c3ccc(SC(F)(F)F)cc3)no2)cn1Cc1ccnc(N2CCNCC2)c1]

A solution of 800 mg (1.72 mmol) of the compound from Example 28A and 2.96 g (34.4 mmol) of piperazine in 17 ml of ethanol was heated in a microwave oven (Biotage Initiator, with dynamic irradiation power control) at 180° C. for 75 min. All volatile components were then substantially removed on a rotary evaporator. About 100 ml of water were added to the residue obtained, and the mixture was extracted two times with in each case about 100 ml of ethyl acetate. The combined organic extracts were washed with saturated sodium chloride solution, dried over anhydrous magnesium sulphate, filtered and freed from the solvent on a rotary evaporator. The crude product was purified by means of preparative HPLC (method 16). Concentration and drying of the product fractions gave 378 mg (43% of theory) of the title compound.

¹H-NMR (400 MHz, DMSO-d₆, δ/ppm): 9.00 (d, 1H), 8.20 (d, 2H), 8.10 (dd, 1H), 8.04 (d, 1H), 7.95 (d, 2H), 6.74 (s, 1H), 6.67 (d, 1H), 6.46 (d, 1H), 5.19 (s, 2H), 3.38-3.34 (m, 4H, partially superposed by the water signal), 2.77-2.74 (m, 4H).

LC/MS (Method 3, ESIpos): Rₜ=0.90 min; m/z=515 [M+H]⁺.

### Example 33A

### 1-{[2-(piperazin-1-yl)pyridin-4-yl]methyl}-5-{3-[4-(1,1,1-trifluoro-2-methylpropan-2-yl)phenyl]-1,2,4-oxadiazol-5-yl}pyridin-2(1H)-one

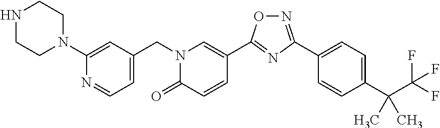 [SMILES: CC(C)(c1ccc(-c2noc(-c3ccc(=O)n(Cc4ccnc(N5CCNCC5)c4)c3)n2)cc1)C(F)(F)F]

Analogously to the process described in Example 31A, 300 mg (0.632 mmol) of the compound from Example 29A and 1.09 g (12.6 mmol) of piperazine were used to obtain 282 mg (70% of theory, purity 82%) of the title compound.

¹H NMR (400 MHz, CDCl₃, δ/ppm): 8.34 (d, 1H), 8.17 (d, 1H), 8.11-8.04 (m, 3H), 7.63 (d, 2H), 6.77 (d, 1H), 6.58 (s, 1H), 6.52 (d, 1H), 5.14 (s, 2H), 3.53-3.49 (m, 4H), 2.99-2.95 (m, 4H), 1.62 (s, 6H).

LC/MS (Method 3, ESIpos): Rₜ=0.95 min; m/z=525 [M+H]⁺.

### Example 34A

### 1-{[2-(piperazin-1-yl)pyridin-4-yl]methyl}-5-(3-{4-[1-(trifluoromethyl)cyclopropyl]phenyl}-1,2,4-oxadiazol-5-yl)pyridin-2(1H)-one

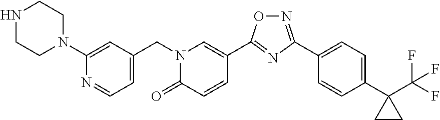 [SMILES: O=c1ccc(-c2nc(-c3ccc(C4(C(F)(F)F)CC4)cc3)no2)cn1Cc1ccnc(N2CCNCC2)c1]

Analogously to the process described in Example 32A, 800 mg (1.69 mmol) of the compound from Example 30A and 2.91 g (33.8 mmol) of piperazine gave 856 mg (94% of theory, purity 97%) of the title compound. In this case, chromatographic purification could be dispensed with.

¹H-NMR (400 MHz, DMSO-d₆, δ/ppm): 8.98 (d, 1H), 8.09 (dd, 1H), 8.08 (d, 2H), 8.04 (d, 1H), 7.68 (d, 2H), 6.74 (s, 1H), 6.66 (d, 1H), 6.46 (d, 1H), 5.19 (s, 2H), 3.38-3.36 (m, 4H), 2.77-2.74 (m, 4H), 1.43-1.40 (m, 2H), 1.22-1.19 (m, 2H).

LC/MS (Method 3, ESIpos): Rₜ=0.85 min; m/z=523 [M+H]⁺.

### Example 35A

### 2-(3-Bromobenzyl)-6-{3-[4-(trifluoromethoxy)phenyl]-1,2,4-oxadiazol-5-yl}pyridazin-3(2H)-one

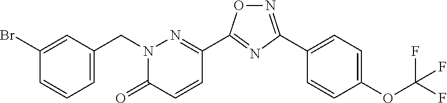 [SMILES: O=c1ccc(-c2nc(-c3ccc(OC(F)(F)F)cc3)no2)nn1Cc1cccc(Br)c1]

Under argon and with ice cooling, 156 mg (3.89 mmol, 60% in mineral oil) of sodium hydride were added to a mixture of 1.05 g (3.24 mmol) of the compound from Example 14A in 20 ml of DMF. After 15 min of stirring at RT, 972 mg (3.89 mmol) of 3-bromobenzyl bromide were added, and the reaction mixture was stirred at RT for a further 60 min. Subsequently, 200 ml each of water and ethyl acetate were added slowly, and the phases were separated. The aqueous phase was extracted once with 100 ml of ethyl acetate, and the combined organic phases were washed once with 200 ml of saturated sodium chloride solution, dried over sodium sulphate, filtered and concentrated. The residue was taken up in dichloromethane and purified by column chromatography (Interchim, silica gel, mobile phase cyclohexane/ethyl acetate 4:1). Drying under high vacuum gave 1.18 g (74% of theory) of the title compound.

¹H NMR (400 MHz, CDCl₃, δ/ppm): 8.21 (d, 2H), 8.05 (d, 1H), 7.66 (s, 1H), 7.46 (d, 2H), 7.37 (d, 2H), 7.23 (t, 1H), 7.10 (d, 1H), 5.43 (s, 2H).

LC/MS (Method 3, ESIpos): Rₜ=1.37 min; m/z=493 [M+H]⁺.

MS (DCI, NH₃): m/z=510 [M+NH₄]⁺.

### Example 36A

### 3-{[6-oxo-3-{3-[4-(trifluoromethoxy)phenyl]-1,2,4-oxadiazol-5-yl}pyridazin-1(6H)-yl]methyl}benzoic acid

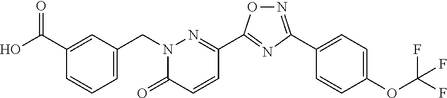 [SMILES: O=C(O)c1cccc(Cn2nc(-c3nc(-c4ccc(OC(F)(F)F)cc4)no3)ccc2=O)c1]

16 ml (15.6 mmol) of 1 M aqueous sodium hydroxide solution were added to a mixture of 1.27 g (1.69 mmol) of the compound from Example 68 in 60 ml of methanol. The mixture was stirred under reflux for 30 min. After cooling to RT, the solvent was removed on a rotary evaporator and 23 ml of 1 M hydrochloric acid were added to the residue. After 15 min of stirring at RT, the solid formed was filtered off and washed twice with in each case 10 ml of water. After drying under a high vacuum, 831 mg (51% of theory, purity 75%) of the title compound were obtained. This material was used without further work-up for subsequent reactions. An aliquot of 80 mg was purified by preparative HPLC (Method 14), giving 38 mg of clean title compound.

¹H-NMR (400 MHz, DMSO-d₆, δ/ppm): 10.1 (br. s, 1H), 8.22 (d, 2H), 8.16 (d, 1H), 7.97 (s, 1H), 7.89 (d, 1H), 7.65-7.60 (m, 3H), 7.51 (t, 1H), 7.26 (d, 1H), 5.49 (s, 2H).

LC/MS (Method 7, ESIpos): Rₜ=1.14 min; m/z=459 [M+H]⁺.

### Example 37A

### 3-{[6-oxo-3-{3-[4-(trifluoromethoxy)phenyl]-1,2,4-oxadiazol-5-yl}pyridazin-1(6H)-yl]methyl}benzoyl chloride

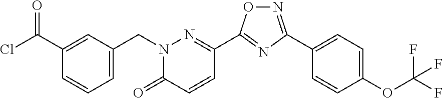 [SMILES: O=C(Cl)c1cccc(Cn2nc(-c3nc(-c4ccc(OC(F)(F)F)cc4)no3)ccc2=O)c1]

At RT, 0.54 ml (6.14 mmol) of oxalyl chloride and a drop of DMF were added to a solution of 750 mg (1.23 mmol, purity 75%) of the compound from Example 36A in 20 ml of dichloromethane. After 2 h of stirring at RT, the solvent was removed on a rotary evaporator. The crude product obtained in this manner was used directly for further reactions.

### Example 38A

### 3-{[6-oxo-3-{3-[4-(1,1,1-trifluoro-2-methylpropan-2-yl)phenyl]-1,2,4-oxadiazol-5-yl}pyridazin-1(6H)-yl]methyl}benzoic acid

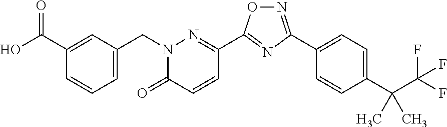 [SMILES: CC(C)(c1ccc(-c2noc(-c3ccc(=O)n(Cc4cccc(C(=O)O)c4)n3)n2)cc1)C(F)(F)F]

19 ml (18.8 mmol) of 1 M aqueous sodium hydroxide solution were added to a mixture of 1.61 g (3.24 mmol) of the compound from Example 69 in 75 ml of methanol. The mixture was stirred under reflux for 30 min. After cooling to RT, the solvent was removed on a rotary evaporator and 15 ml of water and 15 ml of 1 M hydrochloric acid were added to the residue. After 15 min of stirring at RT, the solid formed was filtered off and washed twice with in each case 4 ml of water. After drying under a high vacuum, 1.45 g (73% of theory, purity 79%) of the title compound were obtained. This material was used without further work-up for subsequent reactions. An aliquot of 80 mg was purified by preparative HPLC (Method 14), giving 40 mg of clean title compound.

¹H-NMR (400 MHz, DMSO-d₆, δ/ppm): 13.08 (br. s, 1H), 8.16 (d, 1H), 8.11 (d, 2H), 7.96 (s, 1H), 7.89 (d, 1H), 7.79 (d, 2H), 7.63 (d, 1H), 7.51 (t, 1H), 7.25 (d, 1H), 5.49 (s, 2H), 1.61 (s, 6H).

LC/MS (Method 7, ESIpos): Rₜ=1.19 min; m/z=485 [M+H]⁺.

### Example 39A

### 3-{[6-oxo-3-{3-[4-(1,1,1-trifluoro-2-methylpropan-2-yl)phenyl]-1,2,4-oxadiazol-5-yl}pyridazin-1(6H)-yl]methyl}benzoyl chloride

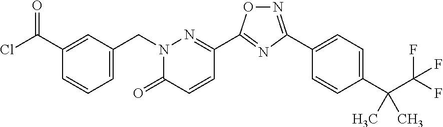 [SMILES: CC(C)(c1ccc(-c2noc(-c3ccc(=O)n(Cc4cccc(C(=O)Cl)c4)n3)n2)cc1)C(F)(F)F]

At RT, 0.9 ml (10.1 mmol) of oxalyl chloride and a few drops of DMF were added to a mixture of 1.25 g (2.01 mmol) of the compound from Example 38A in 30 ml of dichloromethane. After 2 h of stirring at RT, the solvent was removed on a rotary evaporator. The crude product obtained in this manner was used directly for further reactions.

### Example 40A

### 6-{3-[4-(Trifluoromethoxy)phenyl]-1,2,4-oxadiazol-5-yl}-2-[3-(1-{[(triisopropylsilyl)oxy]methyl}cyclopropyl)benzyl]pyridazin-3(2H)-one

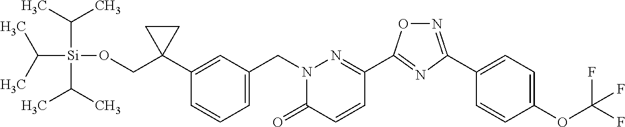 [SMILES: CC(C)[Si](OCC1(c2cccc(Cn3nc(-c4nc(-c5ccc(OC(F)(F)F)cc5)no4)ccc3=O)c2)CC1)(C(C)C)C(C)C]

Analogously to the process described in Example 25A, 139 mg (0.430 mmol) of the compound from Example 14A and 195 mg (0.473 mmol) of the compound from Example 3A gave 161 mg (58% of theory) of the title compound.

¹H NMR (400 MHz, CDCl₃, δ/ppm): 8.20 (d, 2H), 8.02 (d, 1H), 7.51 (s, 1H), 7.39-7.31 (m, 4H), 7.28-7.23 (m, 1H), 7.07 (d, 1H), 5.44 (s, 2H), 3.77 (s, 2H), 1.02-0.92 (m, 21H), 0.92-0.89 (m, 2H), 0.78-0.74 (m, 2H).

LC/MS (Method 7, ESIpos): Rₜ=2.00 min; m/z=641 [M+H]⁺.

### Example 41A

### 6-{3-[4-(1,1,1-Trifluoro-2-methylpropan-2-yl)phenyl]-1,2,4-oxadiazol-5-yl}-2-[3-(1-{[(triisopropylsilyl)oxy]methyl}cyclopropyl)benzyl]pyridazin-3(2H)-one

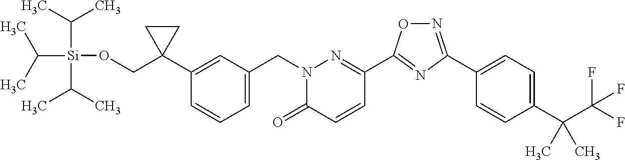 [SMILES: CC(C)[Si](OCC1(c2cccc(Cn3nc(-c4nc(-c5ccc(C(C)(C)C(F)(F)F)cc5)no4)ccc3=O)c2)CC1)(C(C)C)C(C)C]

Analogously to the process described in Example 25A, 151 mg (0.430 mmol) of the compound from Example 15A and 195 mg (0.473 mmol) of the compound from Example 3A gave 178 mg (62% of theory) of the title compound.

¹H NMR (400 MHz, CDCl₃, δ/ppm): 8.14 (d, 2H), 8.04 (d, 1H), 7.65 (d, 2H), 7.51 (s, 1H), 7.37-7.30 (m, 2H), 7.28-7.23 (m, 1H), 7.07 (d, 1H), 5.44 (s, 2H), 3.77 (s, 2H), 1.63 (s, 6H), 1.02-0.92 (m, 21H), 0.92-0.89 (m, 2H), 0.78-0.74 (m, 2H).

LC/MS (Method 4, ESIpos): Rₜ=3.80 min; m/z=667 [M+H]⁺.

### Example 42A

### 2-[(2-Chloropyridin-4-yl)methyl]-6-{3-[4-(trifluoromethoxy)phenyl]-1,2,4-oxadiazol-5-yl}pyridazin-3(2H)-one

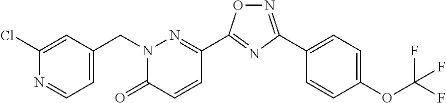 [SMILES: O=c1ccc(-c2nc(-c3ccc(OC(F)(F)F)cc3)no2)nn1Cc1ccnc(Cl)c1]

Analogously to the process described in Example 29A, 324 mg (1.00 mmol) of the compound from Example 14A and 288 mg (1.30 mmol) of the compound from Example 4A gave 292 mg (65% of theory) of the title compound. Here, the reaction time in the alkylation step was 2 h (instead of 30 min), and the chromatographic purification of the crude product was carried out using the mobile phase cyclohexane/ethyl acetate 7:3.

¹H NMR (400 MHz, CDCl₃, δ/ppm): 8.39 (d, 1H), 8.23-8.19 (m, 2H), 8.11 (d, 1H), 7.41-7.35 (m, 3H), 7.32 (dd, 1H), 7.15 (d, 1H), 5.44 (s, 2H).

LC/MS (Method 3, ESIpos): Rₜ=1.17 min; m/z=450 [M+H]⁺.

### Example 43A

### 2-[(2-Chloropyridin-4-yl)methyl]-6-{3-[4-(1,1,1-trifluoro-2-methylpropan-2-yl)phenyl]-1,2,4-oxadiazol-5-yl}pyridazin-3(2H)-one

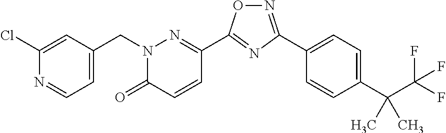 [SMILES: CC(C)(c1ccc(-c2noc(-c3ccc(=O)n(Cc4ccnc(Cl)c4)n3)n2)cc1)C(F)(F)F]

Analogously to the process described in Example 29A, 350 mg (1.00 mmol) of the compound from Example 15A and 288 mg (1.30 mmol) of the compound from Example 4A gave 268 mg (54% of theory, purity 95%) of the title compound. Here, the mobile phase used for the chromatographic purification of the crude product was cyclohexane/ethyl acetate 7:3.

¹H NMR (400 MHz, CDCl₃, δ/ppm): 8.39 (d, 1H), 8.16-8.10 (m, 3H), 7.66 (d, 2H), 7.40 (s, 1H), 7.32 (dd, 1H), 7.15 (d, 1H), 5.44 (s, 2H), 1.63 (s, 6H).

LC/MS (Method 3, ESIpos): Rₜ=1.23 min; m/z=476 [M+H]⁺.

### WORKING EXAMPLES

### Example 1

### 1-(4-Methylbenzyl)-5-{3-[4-(trifluoromethoxy)phenyl]-1,2,4-oxadiazol-5-yl}pyridin-2(1H)-one

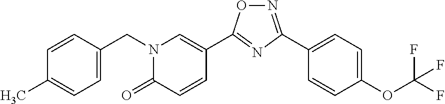 [SMILES: Cc1ccc(Cn2cc(-c3nc(-c4ccc(OC(F)(F)F)cc4)no3)ccc2=O)cc1]

162 mg (1.06 mmol) of 1-hydroxy-1H-benzotriazole hydrate (HOBt) and 203 mg (1.06 mmol) of 1-(3-dimethylaminopropyl)-3-ethylcarbodiimide hydrochloride (EDC) were added to a suspension of 300 mg (1.06 mmol, purity 86%) of the compound from Example 6A (crude product) in 12 ml of DMF, and the mixture was stirred at RT for 30 min. 233 mg (1.06 mmol) of N′-hydroxy-4-(trifluoromethoxy)benzenecarboximidamide were then added, and the mixture was stirred initially at RT for 1 h and then at 150° C. for 2 h. After cooling to RT, 30 ml of ice-water were added and the precipitate formed was filtered off and purified by preparative HPLC (Method 15). This gave 180 mg (36% of theory, purity 90%) of the title compound.

¹H-NMR (400 MHz, DMSO-d₆, δ/ppm): 8.98 (d, 1H), 8.18 (d, 2H), 8.04 (dd, 1H), 7.60 (d, 2H), 7.29 (d, 2H), 7.17 (d, 2H), 6.64 (d, 1H), 5.23 (s, 2H), 2.27 (s, 3H).

LC/MS (Method 3, ESIpos): Rₜ=1.33 min; m/z=428 [M+H]⁺.

### Example 2

### 1-(4-Methylbenzyl)-5-{3-[4-(1,1,1-trifluoro-2-methylpropan-2-yl)phenyl]-1,2,4-oxadiazol-5-yl}pyridin-2(1H)-one

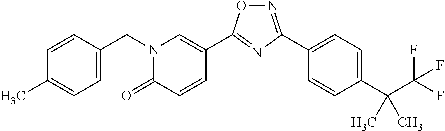 [SMILES: Cc1ccc(Cn2cc(-c3nc(-c4ccc(C(C)(C)C(F)(F)F)cc4)no3)ccc2=O)cc1]

Under argon, 108 mg (0.707 mmol) of 1-hydroxy-1H-benzotriazole hydrate (HOBt) and 136 mg (0.707 mmol) of 1-(3-dimethylaminopropyl)-3-ethylcarbodiimide hydrochloride (EDC) were added to a suspension of 200 mg (0.707 mmol, purity 86%) of the compound from Example 6A (crude product) in 12 ml of DMF, and the mixture was stirred at RT for 30 min. 174 mg (0.707 mmol) of the compound from Example 8A were then added, and the mixture was stirred initially at RT for 30 min and then at 150° C. for 1 h. After cooling to RT, 50 ml of water were added and the mixture was extracted twice with 50 ml of ethyl acetate each time. The combined organic phases were dried over sodium sulphate, filtered and freed from the solvent. The residue was purified by column chromatography (silica gel, mobile phase cyclohexane/ethyl acetate 7:3). This gave 222 mg (69% of theory) of the title compound.

¹H NMR (400 MHz, CDCl₃, δ/ppm): 8.33 (d, 1H), 8.08 (d, 2H), 8.01 (dd, 1H), 7.63 (d, 2H), 7.29 (d, 2H), 7.20 (d, 2H), 6.73 (d, 1H), 5.20 (s, 2H), 2.35 (s, 3H), 1.62 (s, 6H).

LC/MS (Method 3, ESIpos): Rₜ=1.38 min; m/z=454 [M+H]⁺.

### Example 3

### 1-(4-Chlorobenzyl)-5-{3-[4-(trifluoromethoxy)phenyl]-1,2,4-oxadiazol-5-yl}pyridin-2(1H)-one

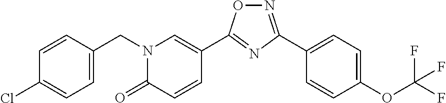 [SMILES: O=c1ccc(-c2nc(-c3ccc(OC(F)(F)F)cc3)no2)cn1Cc1ccc(Cl)cc1]

Under argon and with ice cooling, 21 mg (0.520 mmol, 60% in mineral oil) of sodium hydride were added to a mixture of 129 mg (0.400 mmol) of the compound from Example 10A in 2.5 ml of DMF. After 15 min of stirring at RT, 90 mg (0.440 mmol) of 4-chlorobenzyl bromide were added, and the reaction mixture was stirred at RT overnight. Subsequently, 30 ml each of water and ethyl acetate were added slowly, and the phases were separated. The aqueous phase was extracted with 30 ml of ethyl acetate, and the combined organic phases were washed once with 50 ml of saturated sodium chloride solution, dried over sodium sulphate, filtered and concentrated. The residue was purified by column chromatography (Biotage, silica gel, mobile phase cyclohexane/ethyl acetate 7:3). This gave 139 mg (78% of theory) of the title compound.

¹H NMR (400 MHz, CDCl₃, δ/ppm): 8.35 (d, 1H), 8.15 (d, 2H), 8.02 (dd, 1H), 7.39-7.31 (m, 6H), 6.75 (d, 1H), 5.21 (s, 2H).

LC/MS (Method 3, ESIpos): Rₜ=1.33 min, m/z=448/450 [M+H]⁺.

### Example 4

### 1-(4-Chlorobenzyl)-5-{3-[4-(1,1,1-trifluoro-2-methylpropan-2-yl)phenyl]-1,2,4-oxadiazol-5-yl}pyridin-2(1H)-one

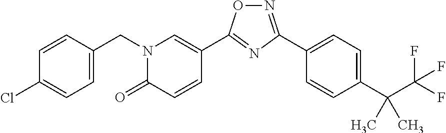 [SMILES: CC(C)(c1ccc(-c2noc(-c3ccc(=O)n(Cc4ccc(Cl)cc4)c3)n2)cc1)C(F)(F)F]

Analogously to the process described in Example 3, 125 mg (0.400 mmol) of the compound from Example 12A and 90 mg (0.440 mmol) of 4-chlorobenzyl bromide gave 125 mg (66% of theory) of the title compound.

¹H NMR (400 MHz, CDCl₃, δ/ppm): 8.35 (d, 1H), 8.09 (d, 2H), 8.03 (dd, 1H), 7.63 (d, 2H), 7.38-7.31 (m, 4H), 6.74 (d, 1H), 5.21 (s, 2H), 1.62 (s, 6H).

LC/MS (Method 3, ESIpos): Rₜ=1.38 min, m/z=474/476 [M+H]⁺.

### Example 5

### 1-(4-Nitrobenzyl)-5-{3-[4-(trifluoromethoxy)phenyl]-1,2,4-oxadiazol-5-yl}pyridin-2(1H)-one

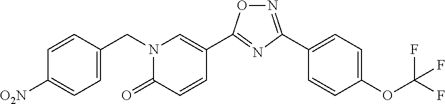 [SMILES: O=c1ccc(-c2nc(-c3ccc(OC(F)(F)F)cc3)no2)cn1Cc1ccc([N+](=O)[O-])cc1]

Analogously to the process described in Example 3, 323 mg (1.00 mmol) of the compound from Example 10A and 238 mg (1.10 mmol) of 4-nitrobenzyl bromide gave 270 mg (59% of theory) of the title compound.

¹H NMR (400 MHz, CDCl₃, δ/ppm): 8.40 (d, 1H), 8.25 (d, 2H), 8.15 (d, 2H), 8.07 (dd, 1H), 7.55 (d, 2H), 7.35 (d, 2H), 6.78 (d, 1H), 5.33 (s, 2H).

LC/MS (Method 3, ESIpos): Rₜ=1.25 min; m/z=459 [M+H]⁺.

### Example 6

### 1-(4-Nitrobenzyl)-5-{3-[4-(1,1,1-trifluoro-2-methylpropan-2-yl)phenyl]-1,2,4-oxadiazol-5-yl}pyridin-2(1H)-one

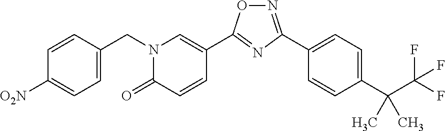 [SMILES: CC(C)(c1ccc(-c2noc(-c3ccc(=O)n(Cc4ccc([N+](=O)[O-])cc4)c3)n2)cc1)C(F)(F)F]

Analogously to the process described in Example 3, 349 mg (1.00 mmol) of the compound from Example 12A and 238 mg (1.10 mmol) of 4-nitrobenzyl bromide gave 361 mg (75% of theory) of the title compound.

¹H NMR (400 MHz, CDCl₃, δ/ppm): 8.40 (d, 1H), 8.25 (d, 2H), 8.08 (m, 3H), 7.63 (d, 2H), 7.55 (d, 2H), 6.78 (d, 1H), 5.33 (s, 2H), 1.63 (s, 6H).

LC/MS (Method 3, ESIpos): Rₜ=1.30 min; m/z=485 [M+H]⁺.

### Example 7

### 1-(4-Aminobenzyl)-5-{3-[4-(trifluoromethoxy)phenyl]-1,2,4-oxadiazol-5-yl}pyridin-2(1H)-one

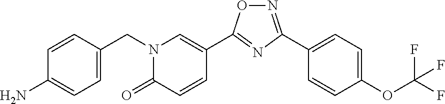 [SMILES: Nc1ccc(Cn2cc(-c3nc(-c4ccc(OC(F)(F)F)cc4)no3)ccc2=O)cc1]

124 mg (1.08 mmol) of indium pellets and 1.3 ml of a saturated aqueous ammonium chloride solution were added to 247 mg (0.539 mmol) of the compound from Example 5 in 10.5 ml of ethanol. The mixture was stirred under reflux for 2 h. A further 0.6 ml of the saturated ammonium chloride solution were then added, and the mixture was stirred under reflux for another 5 h. A further 0.8 ml of the saturated ammonium chloride solution and a spatula tip of indium pellets were added, and the mixture was stirred under reflux for another 10 h. After cooling to RT, 100 ml each of ethyl acetate and water were added to the mixture, and the phases were separated. The aqueous phase was extracted twice with in each case 80 ml of ethyl acetate, and the combined organic phases were dried over sodium sulphate, filtered and concentrated. The residue was dissolved in a mixture of trifluoroacetic acid, DMSO and methanol, filtered again and then purified by preparative HPLC (Method 13). The combined product fractions were concentrated on a rotary evaporator to a residual volume of aqueous phase and adjusted to pH 8 with saturated aqueous sodium carbonate solution. The precipitate formed was filtered off, washed with a little water and dried under high vacuum. This gave 112 mg (49% of theory) of the title compound.

¹H NMR (400 MHz, CDCl₃, δ/ppm): 8.31 (d, 1H), 8.15 (d, 2H), 7.99 (dd, 1H), 7.34 (d, 2H), 7.20 (d, 2H), 6.74-6.66 (m, 3H), 5.11 (s, 2H), 3.76 (br. s, 2H).

LC/MS (Method 3, ESIpos): Rₜ=1.05 min; m/z=429 [M+H]⁺.

### Example 8

### 1-(4-Aminobenzyl)-5-{3-[4-(1,1,1-trifluoro-2-methylpropan-2-yl)phenyl]-1,2,4-oxadiazol-5-yl}pyridin-2(1H)-one

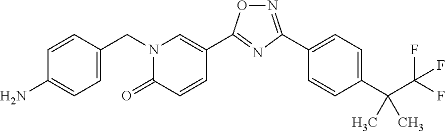 [SMILES: CC(C)(c1ccc(-c2noc(-c3ccc(=O)n(Cc4ccc(N)cc4)c3)n2)cc1)C(F)(F)F]

158 mg (1.37 mmol) of indium pellets and 1.6 ml of a saturated aqueous ammonium chloride solution were added to 333 mg (0.687 mmol) of the compound from Example 6 in 13.5 ml of ethanol. The mixture was stirred under reflux for 2 h. Then, and again an hour later, a further 0.8 ml of the saturated ammonium chloride solution were added. The mixture was then stirred under reflux for another 5 h. A further 0.8 ml of the saturated ammonium chloride solution and a spatula tip of indium pellets were then added, and the mixture was stirred under reflux for another 10 h. After cooling to RT, 100 ml each of ethyl acetate and water were added to the mixture, and the phases were separated. Further work-up was carried out analogously to the process described in Example 7. This gave 125 mg (40% of theory) of the title compound.

¹H NMR (400 MHz, CDCl₃, δ/ppm): 8.32 (d, 1H), 8.08 (d, 2H), 8.00 (dd, 1H), 7.63 (d, 2H), 7.20 (d, 2H), 6.73-6.65 (m, 3H), 5.11 (s, 2H), 3.76 (br. s, 2H), 1.62 (s, 6H).

LC/MS (Method 3, ESIpos): Rₜ=1.11 min; m/z=455 [M+H]⁺.

### Example 9

### 1-(3-{[2-oxo-5-{3-[4-(trifluoromethoxy)phenyl]-1,2,4-oxadiazol-5-yl}pyridin-1(2H)-yl]methyl}phenyl)piperidine-4-carbonitrile

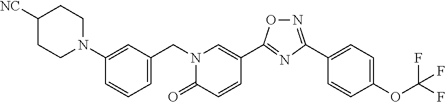 [SMILES: N#CC1CCN(c2cccc(Cn3cc(-c4nc(-c5ccc(OC(F)(F)F)cc5)no4)ccc3=O)c2)CC1]

Under argon, a mixture of 150 mg (0.305 mmol) of the compound from Example 17A, 57 mg (0.518 mmol) of 4-cyanopiperidine, 19 mg (0.020 mmol) of tris(dibenzylideneacetone)dipalladium, 29 mg (0.061 mmol) of 2-dicyclohexylphosphino-2′,4′,6′-triisopropylbiphenyl (X-Phos) and 248 mg (0.762 mmol) of caesium carbonate in 2.4 ml of DMF was stirred in a microwave oven (Biotage Initiator, with dynamic irradiation power control) at 120° C. for 1 h. After cooling to RT, 50 ml each of ethyl acetate and water were added and the mixture was transferred into a separating funnel and extracted. After phase separation, the aqueous phase was extracted twice with 50 ml of ethyl acetate, and the combined organic phases were washed once with saturated sodium chloride solution, dried over sodium sulphate, filtered and concentrated. The residue was purified by means of preparative HPLC (Method 15). The combined product fractions were concentrated to a residual volume of aqueous phase and adjusted to pH 8 with saturated aqueous sodium bicarbonate solution. After two extractions with 50 ml ethyl acetate each, the combined organic phases were dried over sodium sulphate, filtered and concentrated. The residue was re-purified by preparative HPLC (Method 11). Drying under high vacuum gave 15 mg (9% of theory) of the title compound.

¹H NMR (400 MHz, CDCl₃, δ/ppm): 8.34 (d, 1H), 8.15 (d, 2H), 8.02 (dd, 1H), 7.34 (d, 2H), 7.31-7.26 (m, 1H), 6.96 (s, 1H), 6.90 (dd, 1H), 6.86 (d, 1H), 6.75 (d, 1H), 5.19 (s, 2H), 3.47-3.39 (m, 2H), 3.16-3.08 (m, 2H), 2.84-2.76 (m, 1H), 2.11-1.93 (m, 4H).

LC/MS (Method 3, ESIpos): Rₜ=1.25 min; m/z=522 [M+H]⁺.

### Example 10

### 1-(3-{[2-oxo-5-(3-{4-[(trifluoromethyl)sulphanyl]phenyl}-1,2,4-oxadiazol-5-yl)pyridin-1(2H)-yl]methyl}phenyl)piperidine-4-carbonitrile

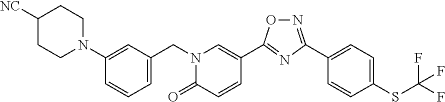 [SMILES: N#CC1CCN(c2cccc(Cn3cc(-c4nc(-c5ccc(SC(F)(F)F)cc5)no4)ccc3=O)c2)CC1]

Under argon, a mixture of 150 mg (0.295 mmol) of the compound from Example 18A, 49 mg (0.443 mmol) of 4-cyanopiperidine, 18 mg (0.020 mmol) of tris(dibenzylideneacetone)dipalladium, 28 mg (0.060 mmol) of 2-dicyclohexylphosphino-2′,4′,6′-triisopropylbiphenyl (X-Phos) and 57 mg (0.590 mmol) of sodium tert-butoxide in 3 ml of toluene was stirred in a microwave oven (Biotage Initiator, with dynamic irradiation power control) at 80° C. for 2 h. After cooling to RT, the mixture was diluted with about 50 ml of ethyl acetate and washed successively with in each case about 50 ml of water and saturated sodium chloride solution. After drying over anhydrous magnesium sulphate, the mixture was filtered and the solvent was removed on a rotary evaporator. The residue that remained was purified by means of preparative HPLC (Method 16). The combined product fractions were concentrated to dryness. This gave 21 mg (13% of theory) of the title compound.

¹H-NMR (400 MHz, DMSO-d₆, δ/ppm): 8.96 (d, 1H), 8.19 (d, 2H), 8.06 (dd, 1H), 7.94 (d, 2H), 7.19 (t, 1H), 7.05 (s, 1H), 6.90 (d, 1H), 6.76 (d, 1H), 6.66 (d, 1H), 5.21 (s, 2H), 3.38-3.31 (m, 2H, partially superimposed by the water signal), 3.08-3.01 (m, 3H), 2.01-1.94 (m, 2H), 1.84-1.79 (m, 2H).

LC/MS (Method 7, ESIpos): Rₜ=1.35 min; m/z=538 [M+H]⁺.

### Example 11

### 1-(3-{[2-oxo-5-{3-[4-(1,1,1-trifluoro-2-methylpropan-2-yl)phenyl]-1,2,4-oxadiazol-5-yl}pyridin-1(2H)-yl]methyl}phenyl)piperidine-4-carbonitrile

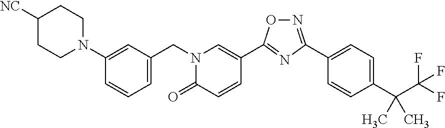 [SMILES: CC(C)(c1ccc(-c2noc(-c3ccc(=O)n(Cc4cccc(N5CCC(C#N)CC5)c4)c3)n2)cc1)C(F)(F)F]

Analogously to the process described in Example 9, 130 mg (0.251 mmol) of the compound from Example 19A and 47 mg (0.426 mmol) of 4-cyanopiperidine gave 28 mg (21% of theory) of the title compound.

¹H NMR (400 MHz, CDCl₃, δ/ppm): 8.34 (d, 1H), 8.08 (d, 2H), 8.03 (dd, 1H), 7.63 (d, 2H), 7.30-7.27 (m, 1H), 6.96 (s, 1H), 6.90 (dd, 1H), 6.86 (d, 1H), 6.74 (d, 1H), 5.19 (s, 2H), 3.47-3.39 (m, 2H), 3.16-3.07 (m, 2H), 2.84-2.75 (m, 1H), 2.11-1.93 (m, 4H), 1.61 (s, 6H).

LC/MS (Method 3, ESIpos): Rₜ=1.32 min; m/z=548 [M+H]⁺.

### Example 12

### 1-(3-{[2-oxo-5-(3-{4-[1-(trifluoromethyl)cyclopropyl]phenyl}-1,2,4-oxadiazol-5-yl)pyridin-1(2H)-yl]methyl}phenyl)piperidine-4-carbonitrile

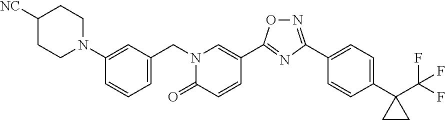 [SMILES: N#CC1CCN(c2cccc(Cn3cc(-c4nc(-c5ccc(C6(C(F)(F)F)CC6)cc5)no4)ccc3=O)c2)CC1]

Analogously to the process described in Example 10, 150 mg (0.291 mmol) of the compound from Example 20A and 48 mg (0.436 mmol) of 4-cyanopiperidine gave 33 mg (21% of theory) of the title compound. In this case, the reaction mixture was, prior to aqueous work-up, diluted not with ethyl acetate but with dichloromethane.

¹H NMR (400 MHz, CDCl₃, δ/ppm): 8.33 (d, 1H), 8.07 (d, 2H), 8.02 (dd, 1H), 7.59 (d, 2H), 7.28 (t, 1H, partially superimposed by the CHCl₃ signal), 6.96 (s, 1H), 6.90 (d, 1H), 6.86 (d, 1H), 6.74 (d, 1H), 5.19 (s, 2H), 3.46-3.40 (m, 2H), 3.15-3.09 (m, 2H), 2.83-2.77 (m, 1H), 2.10-1.95 (m, 4H), 1.43-1.40 (m, 2H), 1.10-1.06 (m, 2H).

LC/MS (Method 7, ESIpos): Rₜ=1.33 min; m/z=546 [M+H]⁺.

### Example 13

### 1-[3-(3-Hydroxyazetidin-1-yl)benzyl]-5-{3-[4-(trifluoromethoxy)phenyl]-1,2,4-oxadiazol-5-yl}pyridin-2(1H)-one

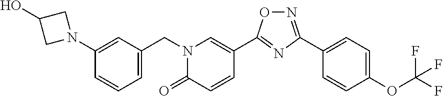 [SMILES: O=c1ccc(-c2nc(-c3ccc(OC(F)(F)F)cc3)no2)cn1Cc1cccc(N2CC(O)C2)c1]

A mixture of 492 mg (1.00 mmol) of the compound from Example 17A, 530 mg (1.70 mmol) of the compound from Example 16A, 61 mg (0.067 mmol) of tris(dibenzylideneacetone)dipalladium, 95 mg (0.200 mmol) of 2-dicyclohexylphosphino-2′,4′,6′-triisopropylbiphenyl (X-Phos) and 240 mg (2.50 mmol) of sodium tert-butoxide in 8 ml of DMF was stirred at 120° C. under argon in a microwave oven (Biotage Initiator, with dynamic control of the incident power) for 1 h. After cooling to RT, 100 ml each of ethyl acetate and water were added and the mixture was transferred into a separating funnel and extracted. After phase separation, the aqueous phase was extracted twice with 70 ml of ethyl acetate, and the combined organic phases were washed once with 100 ml of saturated sodium chloride solution, dried over sodium sulphate, filtered and concentrated. The residue was dissolved in 24 ml of THF, and 2.4 ml of a 1 M solution of tetra-n-butylammonium fluoride in THF were added. After 5 h of stirring at RT, 100 ml each of ethyl acetate and water were added and the mixture was transferred into a separating funnel and extracted. After phase separation, the aqueous phase was extracted once with 50 ml of ethyl acetate, and the combined organic phases were dried over sodium sulphate, filtered and concentrated. The residue was initially triturated with dichloromethane, then purified by column chromatography (silica gel, mobile phase cyclohexane/ethyl acetate 1:1) and finally re-purified by preparative HPLC (Method 18). Drying under high vacuum gave 12 mg (2.5% of theory) of the title compound.

¹H NMR (400 MHz, CDCl₃, δ/ppm): 8.32 (d, 1H), 8.14 (d, 2H), 8.00 (dd, 1H), 7.34 (d, 2H), 7.25-7.19 (m, 1H), 6.76-6.69 (m, 2H), 6.46-6.43 (m, 2H), 5.17 (s, 2H), 4.76 (br. s, 1H), 4.21-4.15 (m, 2H), 3.70 (dd, 2H), 2.26 (br. s, 1H).

LC/MS (Method 1, ESIpos): Rₜ=1.35 min; m/z=485 [M+H]⁺.

### Example 14

### 1-[3-(3-Hydroxyazetidin-1-yl)benzyl]-5-(3-{4-[(trifluoromethyl)sulphanyl]phenyl}-1,2,4-oxadiazol-5-yl)pyridin-2(1H)-one

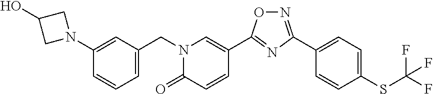 [SMILES: O=c1ccc(-c2nc(-c3ccc(SC(F)(F)F)cc3)no2)cn1Cc1cccc(N2CC(O)C2)c1]

A mixture of 200 mg (0.393 mmol) of the compound from Example 18A, 184 mg (0.590 mmol) of the compound from Example 16A, 24 mg (0.026 mmol) of tris(dibenzylideneacetone)dipalladium, 38 mg (0.079 mmol) of 2-dicyclohexylphosphino-2′,4′,6′-triisopropylbiphenyl (X-Phos) and 76 mg (0.787 mmol) of sodium tert-butoxide in 4 ml of toluene was stirred at 80° C. under argon in a microwave oven (Biotage Initiator, with dynamic control of the incident power) for 3 h. After cooling to RT, the mixture was diluted with about 50 ml of ethyl acetate and washed successively with in each case about 50 ml of water and saturated sodium chloride solution. After drying over anhydrous magnesium sulphate, the mixture was filtered and the solvent was removed on a rotary evaporator. The remaining residue was dissolved in 5 ml of THF, and 393 μl (0.393 mmol) of a 1 M solution of tetra-n-butylammonium fluoride in THF were added. After 2 h of stirring at RT, the reaction mixture was diluted with about 5 ml of methanol and separated completely into its components by means of preparative HPLC (Method 16). The product fractions were combined and concentrated to dryness on a rotary evaporator. The solid obtained was stirred in a solvent mixture of 6 ml of pentane and 2 ml of diisopropyl ether at RT overnight. The solid was then filtered off with suction and washed with a little pentane. For conversion into the salt-free form (free base), the solid was dissolved in about 5 ml of methanol and passed over a bicarbonate cartridge (from Polymerlabs, Stratospheres SPE, PL-HCO₃ MP SPE, capacity 0.9 mmol). Concentration and drying under a high vacuum gave 22 mg (11% of theory) of the title compound.

¹H NMR (400 MHz, CDCl₃, δ/ppm): 8.32 (d, 1H), 8.15 (d, 2H), 8.01 (dd, 1H), 7.78 (d, 2H), 7.23 (dd, 1H), 6.74 (d, 1H), 6.72 (d, 1H), 6.45 (d+s, tog. 2H), 5.17 (s, 2H), 4.80-4.73 (m, 1H), 4.19 (dd, 2H), 3.69 (dd, 2H), 2.21-2.18 (m, 1H).

LC/MS (Method 7, ESIpos): Rₜ=1.22 min; m/z=501 [M+H]⁺.

### Example 15

### 1-[3-(3-Hydroxyazetidin-1-yl)benzyl]-5-{3-[4-(1,1,1-trifluoro-2-methylpropan-2-yl)phenyl]-1,2,4-oxadiazol-5-yl}pyridin-2(1H)-one

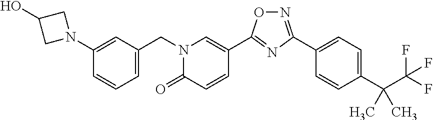 [SMILES: CC(C)(c1ccc(-c2noc(-c3ccc(=O)n(Cc4cccc(N5CC(O)C5)c4)c3)n2)cc1)C(F)(F)F]

Analogously to the process described in Example 13, 130 mg (0.251 mmol) of the compound from Example 19A and 133 mg (0.426 mmol) of the compound from Example 16A gave 7 mg (5% of theory) of the title compound. Here, the reaction time in the second part reaction (cleavage of the silyl ether with TBAF) was 2 h (instead of 5 h). Initial trituration of the isolated crude product with dichloromethane was dispensed with, and for the final purification by preparative HPLC, Method 11 was employed.

¹H NMR (400 MHz, CDCl₃, δ/ppm): 8.32 (d, 1H), 8.08 (d, 2H), 8.01 (dd, 1H), 7.63 (d, 2H), 7.25-7.18 (m, 1H), 6.78-6.66 (m, 2H), 6.48-6.40 (m, 2H), 5.16 (s, 2H), 4.80-4.69 (m, 1H), 4.18 (t, 2H), 3.69 (dd, 2H), 2.29 (br. s, 1H), 1.62 (s, 6H).

LC/MS (Method 3, ESIpos): Rₜ=1.20 min; m/z=511 [M+H]⁺.

### Example 16

### 1-[3-(3-Hydroxyazetidin-1-yl)benzyl]-5-(3-{4-[1-(trifluoromethyl)cyclopropyl]phenyl}-1,2,4-oxadiazol-5-yl)pyridin-2(1H)-one

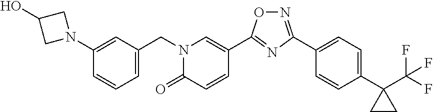 [SMILES: O=c1ccc(-c2nc(-c3ccc(C4(C(F)(F)F)CC4)cc3)no2)cn1Cc1cccc(N2CC(O)C2)c1]

Analogously to the process described in Example 14, 200 mg (0.387 mmol) of the compound from Example 20A and 181 mg (0.581 mmol) of the compound from Example 16A gave 20 mg (10% of theory, 97% pure) of the title compound. Here, after purification by preparative HPLC, the product was not triturated with pentane/diisopropyl ether, but there was a second purification by preparative HPLC (Method 12).

¹H NMR (400 MHz, CDCl₃, δ/ppm): 8.31 (d, 1H), 8.07 (d, 2H), 8.01 (dd, 1H), 7.58 (d, 2H), 7.22 (dd, 1H), 6.73 (d, 1H), 6.71 (d, 1H), 6.44 (d+s, tog. 2H), 5.16 (s, 2H), 4.80-4.72 (m, 1H), 4.18 (dd, 2H), 3.69 (dd, 2H), 2.06-2.02 (m, 1H), 1.43-1.40 (m, 2H), 1.09-1.06 (m, 2H).

LC/MS (Method 3, ESIpos): Rₜ=1.17 min; m/z=509 [M+H]⁺.

### Example 17

### tert-Butyl 4-(3-{[2-oxo-5-{3-[4-(1,1,1-trifluoro-2-methylpropan-2-yl)phenyl]-1,2,4-oxadiazol-5-yl}pyridin-1(2H)-yl]methyl}phenyl)piperazine-1-carboxylate

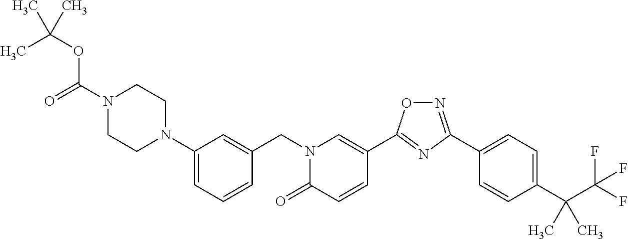 [SMILES: CC(C)(C)OC(=O)N1CCN(c2cccc(Cn3cc(-c4nc(-c5ccc(C(C)(C)C(F)(F)F)cc5)no4)ccc3=O)c2)CC1]

Under argon, 93 mg (0.502 mmol) of tert-butyl piperazine-1-carboxylate, 11 mg (0.050 mmol) of palladium(II) acetate, 15 mg (0.050 mmol) of 2-(di-tert-butylphosphino)biphenyl and 204 mg (0.627 mmol) of caesium carbonate were added to a mixture of 130 mg (0.251 mmol) of the compound from Example 19A in 5.5 ml of toluene. The mixture was stirred under reflux for 23 h. After cooling to RT, the reaction mixture was filtered through Celite and the filtrate was concentrated. The residue was purified by column chromatography (Biotage, silica gel, mobile phase cyclohexane/ethyl acetate 7:3). Drying under high vacuum gave 9 mg (6% of theory) of the title compound.

¹H NMR (400 MHz, CDCl₃, δ/ppm): 8.34 (d, 1H), 8.08 (d, 2H), 8.02 (dd, 1H), 7.63 (d, 2H), 7.31-7.27 (m, 1H), 6.95 (s, 1H), 6.90 (dd, 1H), 6.86 (d, 1H), 6.74 (d, 1H), 5.19 (s, 2H), 3.59-3.55 (m, 4H), 3.17-3.12 (m, 4H), 1.63 (s, 6H), 1.48 (s, 9H).

LC/MS (Method 3, ESIpos): Rₜ=1.46 min; m/z=624 [M+H]⁺.

### Example 18

### Methyl 3-{[2-oxo-5-{3-[4-(trifluoromethoxy)phenyl]-1,2,4-oxadiazol-5-yl}pyridin-1(2H)-yl]methyl}benzoate

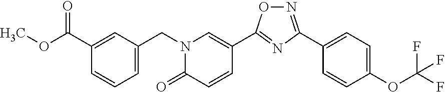 [SMILES: COC(=O)c1cccc(Cn2cc(-c3nc(-c4ccc(OC(F)(F)F)cc4)no3)ccc2=O)c1]

Under argon and with ice cooling, 169 mg (4.22 mmol, 60% in mineral oil) of sodium hydride were added to a mixture of 1.05 g (3.25 mmol) of the compound from Example 10A in 20 ml of DMF. After 15 min of stirring at RT, 819 mg (3.57 mmol) of methyl 3-(bromomethyl)benzoate were added, and the reaction mixture was stirred at RT for 2 h. Subsequently, 200 ml each of water and ethyl acetate were added slowly, and the phases were separated. The aqueous phase was extracted twice with in each case 150 ml of ethyl acetate, and the combined organic phases were dried over sodium sulphate, filtered and concentrated. The residue was stirred with about 15 ml of a hot 1:1 mixture of ethyl acetate and cyclohexane. The solid was filtered off with suction and washed twice with in each case 2 ml of cyclohexane/ethyl acetate 1:1. This gave, after drying under high vacuum, a first batch of the title compound. The filtrate and the wash solutions were combined and concentrated, and the residue was purified by column chromatography (Biotage, silica gel, mobile phase cyclohexane/ethyl acetate 7:3), giving a second batch of the title compound. A total of 1.28 g (84% of theory) of the title compound were obtained in this manner.

¹H NMR (400 MHz, CDCl₃, δ/ppm): 8.38 (d, 1H), 8.15 (d, 2H), 8.06-8.01 (m, 3H), 7.60 (d, 1H), 7.51-7.45 (m, 1H), 7.34 (d, 2H), 6.76 (d, 1H), 5.29 (s, 2H), 3.93 (s, 3H).

LC/MS (Method 3, ESIpos): Rₜ=1.24 min; m/z=472 [M+H]⁺.

### Example 19

### Methyl 3-{[2-oxo-5-{3-[4-(1,1,1-trifluoro-2-methylpropan-2-yl)phenyl]-1,2,4-oxadiazol-5-yl}pyridin-1(2H)-yl]methyl}benzoate

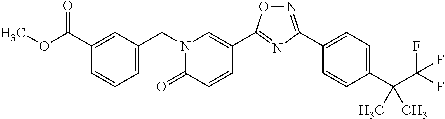 [SMILES: COC(=O)c1cccc(Cn2cc(-c3nc(-c4ccc(C(C)(C)C(F)(F)F)cc4)no3)ccc2=O)c1]

Analogously to the process described in Example 18, 1.05 g (3.00 mmol) of the compound from Example 12A and 756 mg (3.30 mmol) of methyl 3-(bromomethyl)benzoate gave 971 mg (65% of theory) of the title compound. Here, chromatographic work-up of the mother liquor and the wash solutions from the trituration with cyclohexane/ethyl acetate could be dispensed with.

¹H NMR (400 MHz, CDCl₃, δ/ppm): 8.38 (d, 1H), 8.09 (d, 2H), 8.07-8.00 (m, 3H), 7.65-7.58 (m, 3H), 7.50-7.45 (m, 1H), 6.76 (d, 1H), 5.29 (s, 2H), 3.93 (s, 3H), 1.62 (s, 6H).

LC/MS (Method 3, ESIpos): Rₜ=1.31 min; m/z=498 [M+H]⁺.

### Example 20

### N-Methyl-3-{[2-oxo-5-{3-[4-(trifluoromethoxy)phenyl]-1,2,4-oxadiazol-5-yl}pyridin-1(2H)-yl]methyl}benzamide

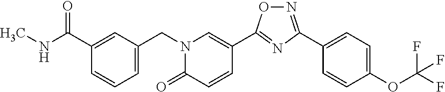 [SMILES: CNC(=O)c1cccc(Cn2cc(-c3nc(-c4ccc(OC(F)(F)F)cc4)no3)ccc2=O)c1]

70 μl (0.400 mmol) of N,N-diisopropylethylamine and 95 mg (0.200 mmol) of the compound from Example 22A, dissolved in 1.5 ml THF, were added to a solution of 150 μl (0.300 mmol) of methylamine (as a 2 M solution in THF) in 1.5 ml of THF. The mixture was stirred at RT for 1 h. 5 ml of water were then added to the reaction mixture. The resulting solid was filtered off and washed twice with 2 ml of water each time. Drying under high vacuum gave 84 mg (89% of theory) of the title compound.

¹H NMR (400 MHz, CDCl₃, δ/ppm): 8.39 (d, 1H), 8.16 (d, 2H), 8.03 (dd, 1H), 7.82 (s, 1H), 7.72 (d, 1H), 7.53 (d, 1H), 7.46 (t, 1H), 7.34 (d, 2H), 6.75 (d, 1H), 6.21 (br. s, 1H), 5.27 (s, 2H), 3.01 (d, 3H).

LC/MS (Method 7, ESIpos): Rₜ=1.09 min; m/z=471 [M+H]⁺.

### Example 21

### N-Methyl-3-{[2-oxo-5-{3-[4-(1,1,1-trifluoro-2-methylpropan-2-yl)phenyl]-1,2,4-oxadiazol-5-yl}pyridin-1(2H)-yl]methyl}benzamide

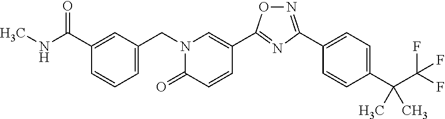 [SMILES: CNC(=O)c1cccc(Cn2cc(-c3nc(-c4ccc(C(C)(C)C(F)(F)F)cc4)no3)ccc2=O)c1]

Analogously to the process described under Example 20, 93 mg (0.185 mmol) of the compound from Example 24A and 139 μl (0.278 mmol) of methylamine (as a 2 M solution in THF) gave 87 mg (95% of theory) of the title compound.

¹H NMR (400 MHz, CDCl₃, δ/ppm): 8.39 (d, 1H), 8.09 (d, 2H), 8.04 (dd, 1H), 7.82 (s, 1H), 7.72 (d, 1H), 7.63 (d, 2H), 7.53 (d, 1H), 7.46 (t, 1H), 6.74 (d, 1H), 6.22 (br. s, 1H), 5.27 (s, 2H), 3.01 (d, 3H), 1.62 (s, 6H).

LC/MS (Method 3, ESIpos): Rₜ=1.14 min; m/z=497 [M+H]⁺.

### Example 22

### N,N-Dimethyl-3-{[2-oxo-5-{3-[4-(trifluoromethoxy)phenyl]-1,2,4-oxadiazol-5-yl}pyridin-1(2H)-yl]methyl}benzamide

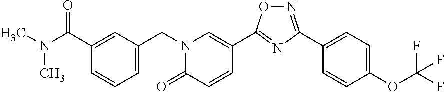 [SMILES: CN(C)C(=O)c1cccc(Cn2cc(-c3nc(-c4ccc(OC(F)(F)F)cc4)no3)ccc2=O)c1]

70 μl (0.400 mmol) of N,N-diisopropylethylamine and 95 mg (0.200 mmol) of the compound from Example 22A, dissolved in 1.5 ml THF, were added to a solution of 150 μl (0.300 mmol) of dimethylamine (as a 2 M solution in THF) in 1.5 ml of THF. The mixture was stirred at RT for 1 h. 30 ml of ethyl acetate were then added, and the reaction mixture was washed once with 50 ml of water. The aqueous phase was reextracted once with 30 ml of ethyl acetate. The combined organic phases were dried over sodium sulphate, filtered and concentrated on a rotary evaporator. The residue was purified by column chromatography (Biotage, silica gel, mobile phase dichloromethane/methanol 95:5). Drying under high vacuum gave 40 mg (40% of theory) of the title compound.

¹H NMR (400 MHz, CDCl₃, δ/ppm): 8.48 (d, 1H), 8.16 (d, 2H), 8.03 (dd, 1H), 7.47-7.42 (m, 3H), 7.42-7.32 (m, 3H), 6.73 (d, 1H), 5.29 (s, 2H), 3.10 (s, 3H), 2.97 (s, 3H).

LC/MS (Method 7, ESIpos): Rₜ=1.13 min; m/z=485 [M+H]⁺.

### Example 23

### N,N-Dimethyl-3-{[2-oxo-5-{3-[4-(1,1,1-trifluoro-2-methylpropan-2-yl)phenyl]-1,2,4-oxadiazol-5-yl}pyridin-1(2H)-yl]methyl}benzamide

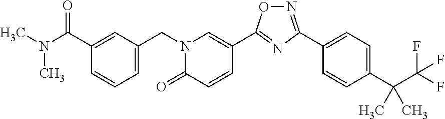 [SMILES: CN(C)C(=O)c1cccc(Cn2cc(-c3nc(-c4ccc(C(C)(C)C(F)(F)F)cc4)no3)ccc2=O)c1]

Analogously to the process described under Example 22, 93 mg (0.185 mmol) of the compound from Example 24A and 139 μl (0.278 mmol) of dimethylamine (as a 2 M solution in THF) gave 51 mg (54% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.04 (dd, 1H), 7.63 (d, 2H), 7.46-7.39 (m, 4H), 6.74 (d, 1H), 5.26 (s, 2H), 3.10 (s, 3H), 2.97 (s, 3H), 1.62 (s, 6H).

LC/MS (Method 7, ESIpos): Rₜ=1.18 min; m/z=511 [M+H]⁺.

### Example 24

### 1-[3-(Azetidin-1-ylcarbonyl)benzyl]-5-{3-[4-(trifluoromethoxy)phenyl]-1,2,4-oxadiazol-5-yl}pyridin-2(1H)-one

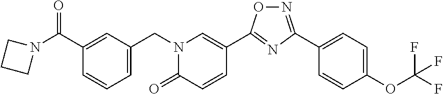 [SMILES: O=C(c1cccc(Cn2cc(-c3nc(-c4ccc(OC(F)(F)F)cc4)no3)ccc2=O)c1)N1CCC1]

Analogously to the process described under Example 22, 95 mg (0.200 mmol) of the compound from Example 22A and 20 μl (0.300 mmol) of azetidine gave 76 mg (76% of theory) of the title compound. Here, the mobile phase used for chromatographic purification was dichloromethane/methanol 80:1.

¹H NMR (400 MHz, CDCl₃, δ/ppm): 8.39 (d, 1H), 8.16 (d, 2H), 8.03 (dd, 1H), 7.69 (s, 1H), 7.58 (d, 1H), 7.49 (d, 1H), 7.44 (t, 1H), 7.34 (d, 2H), 6.75 (d, 1H), 5.26 (s, 2H), 4.30 (t, 2H), 4.22 (t, 2H), 2.34 (quint, 2H).

LC/MS (Method 7, ESIpos): Rₜ=1.14 min; m/z=497 [M+H]⁺.

### Example 25

### 1-[3-(Azetidin-1-ylcarbonyl)benzyl]-5-{3-[4-(1,1,1-trifluoro-2-methylpropan-2-yl)phenyl]-1,2,4-oxadiazol-5-yl}pyridin-2(1H)-one

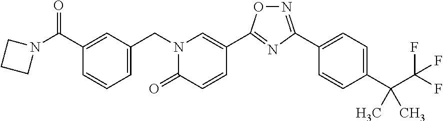 [SMILES: CC(C)(c1ccc(-c2noc(-c3ccc(=O)n(Cc4cccc(C(=O)N5CCC5)c4)c3)n2)cc1)C(F)(F)F]

Analogously to the process described under Example 22, 93 mg (0.185 mmol) of the compound from Example 24A and 19 μl (0.278 mmol) of azetidine gave 71 mg (73% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.04 (dd, 1H), 7.68 (s, 1H), 7.63 (d, 2H), 7.59 (d, 1H), 7.48 (d, 1H), 7.44 (t, 1H), 6.75 (d, 1H), 5.26 (s, 2H), 4.28 (t, 2H), 4.22 (t, 2H), 2.34 (quint, 2H), 1.62 (s, 6H).

LC/MS (Method 7, ESIpos): Rₜ=1.19 min; m/z=523 [M+H]⁺.

### Example 26

### 1-{3-[(3-Hydroxyazetidin-1-yl)carbonyl]benzyl}-5-{3-[4-(trifluoromethoxy)phenyl]-1,2,4-oxadiazol-5-yl}pyridin-2(1H)-one

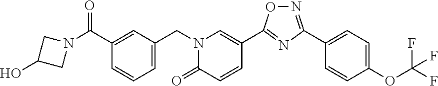 [SMILES: O=C(c1cccc(Cn2cc(-c3nc(-c4ccc(OC(F)(F)F)cc4)no3)ccc2=O)c1)N1CC(O)C1]

Analogously to the process described in Example 22, 95 mg (0.200 mmol) of the compound from Example 22A and 33 mg (0.300 mmol) of 3-azetidinol hydrochloride gave 83 mg (81% of theory) of the title compound.

¹H NMR (400 MHz, CDCl₃, δ/ppm): 8.41 (d, 1H), 8.16 (d, 2H), 8.03 (dd, 1H), 7.69 (s, 1H), 7.57 (d, 1H), 7.50 (d, 1H), 7.43 (t, 1H), 7.34 (d, 2H), 6.75 (d, 1H), 5.26 (s, 2H), 4.75-4.66 (m, 1H), 4.45 (ddd, 2H), 4.20-4.13 (m, 1H), 4.09-4.01 (m, 1H), 2.76 (d, 1H).

LC/MS (Method 7, ESIpos): Rₜ=1.02 min; m/z=513 [M+H]⁺.

### Example 27

### 1-{3-[(3-Hydroxyazetidin-1-yl)carbonyl]benzyl}-5-{3-[4-(1,1,1-trifluoro-2-methylpropan-2-yl)phenyl]-1,2,4-oxadiazol-5-yl}pyridin-2(1H)-one

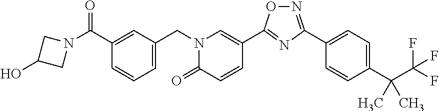 [SMILES: CC(C)(c1ccc(-c2noc(-c3ccc(=O)n(Cc4cccc(C(=O)N5CC(O)C5)c4)c3)n2)cc1)C(F)(F)F]

Analogously to the process described in Example 22, 93 mg (0.185 mmol) of the compound from Example 24A and 30 mg (0.278 mmol) of 3-azetidinol hydrochloride gave 82 mg (82% of theory) of the title compound. Here, the mobile phase used for chromatographic purification was dichloromethane/methanol 40:1.

¹H NMR (400 MHz, CDCl₃, δ/ppm): 8.41 (d, 1H), 8.09 (d, 2H), 8.04 (dd, 1H), 7.68 (s, 1H), 7.63 (d, 2H), 7.58 (d, 1H), 7.49 (d, 1H), 7.43 (t, 1H), 6.74 (d, 1H), 5.26 (s, 2H), 4.75-4.66 (m, 1H), 4.45 (ddd, 2H), 4.20-4.13 (m, 1H), 4.09-4.01 (m, 1H), 2.70 (d, 1H), 1.63 (s, 6H).

LC/MS (Method 7, ESIpos): Rₜ=1.07 min; m/z=539 [M+H]⁺.

### Example 28

### 1-[3-(Pyrrolidin-1-ylcarbonyl)benzyl]-5-{3-[4-(trifluoromethoxy)phenyl]-1,2,4-oxadiazol-5-yl}pyridin-2(1H)-one

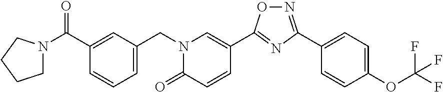 [SMILES: O=C(c1cccc(Cn2cc(-c3nc(-c4ccc(OC(F)(F)F)cc4)no3)ccc2=O)c1)N1CCCC1]

Analogously to the process described under Example 22, 119 mg (0.250 mmol) of the compound from Example 22A and 31 μl (0.300 mmol) of pyrrolidine gave 107 mg (84% of theory) of the title compound.

¹H NMR (400 MHz, CDCl₃, δ/ppm): 8.38 (d, 1H), 8.16 (d, 2H), 8.03 (dd, 1H), 7.55 (s, 1H), 7.51-7.47 (m, 1H), 7.46-7.41 (m, 2H), 7.34 (d, 2H), 6.75 (d, 1H), 5.26 (s, 2H), 3.63 (t, 2H), 3.41 (t, 2H), 2.00-1.92 (m, 2H), 1.91-1.83 (m, 2H).

LC/MS (Method 7, ESIpos): Rₜ=1.18 min; m/z=511 [M+H]⁺.

### Example 29

### 1-[3-(Pyrrolidin-1-ylcarbonyl)benzyl]-5-{3-[4-(1,1,1-trifluoro-2-methylpropan-2-yl)phenyl]-1,2,4-oxadiazol-5-yl}pyridin-2(1H)-one

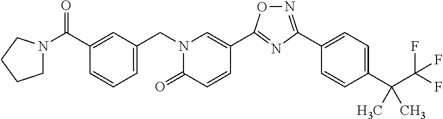 [SMILES: CC(C)(c1ccc(-c2noc(-c3ccc(=O)n(Cc4cccc(C(=O)N5CCCC5)c4)c3)n2)cc1)C(F)(F)F]

70 μl (0.400 mmol) of N,N-diisopropylethylamine and 100 mg (0.200 mmol) of the compound from Example 24A, dissolved in 2 ml THF, were added to a solution of 25 μl (0.300 mmol) of pyrrolidine in 2 ml of THF. The mixture was stirred at RT for 1 h. 30 ml of ethyl acetate were then added, and the reaction mixture was washed once with 50 ml of water. The aqueous phase was reextracted once with 30 ml of ethyl acetate. The combined organic phases were dried over sodium sulphate, filtered and concentrated on a rotary evaporator. Drying under high vacuum gave 96 mg (89% of theory) of the title compound.

¹H NMR (400 MHz, CDCl₃, δ/ppm): 8.39 (d, 1H), 8.09 (d, 2H), 8.04 (dd, 1H), 7.63 (d, 2H), 7.55 (s, 1H), 7.51-7.47 (m, 1H), 7.46-7.40 (m, 2H), 6.74 (d, 1H), 5.26 (s, 2H), 3.63 (t, 2H), 3.41 (t, 2H), 2.00-1.91 (m, 2H), 1.91-1.82 (m, 2H), 1.66 (s, 6H).

LC/MS (Method 3, ESIpos): Rₜ=1.22 min; m/z=537 [M+H]⁺.

### Example 30

### 1-[3-(1,2-Oxazolidin-2-ylcarbonyl)benzyl]-5-{3-[4-(trifluoromethoxy)phenyl]-1,2,4-oxadiazol-5-yl}pyridin-2(1H)-one

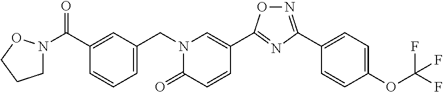 [SMILES: O=C(c1cccc(Cn2cc(-c3nc(-c4ccc(OC(F)(F)F)cc4)no3)ccc2=O)c1)N1CCCO1]

Analogously to the process described in Example 22, 119 mg (0.250 mmol) of the compound from Example 22A and 27 mg (0.375 mmol) of isoxazolidine gave 90 mg (70% of theory) of the title compound. Here, the mobile phase used for chromatographic purification was dichloromethane/methanol 40:1.

¹H NMR (400 MHz, CDCl₃, δ/ppm): 8.38 (d, 1H), 8.16 (d, 2H), 8.02 (dd, 1H), 7.82-7.77 (m, 2H), 7.51 (d, 1H), 7.44 (t, 1H), 7.34 (d, 2H), 6.75 (d, 1H), 5.28 (s, 2H), 3.97 (t, 2H), 3.90 (t, 2H), 2.36 (quint, 2H).

LC/MS (Method 7, ESIpos): Rₜ=1.34 min; m/z=513 [M+H]⁺.

### Example 31

### 1-[3-(1,2-Oxazolidin-2-ylcarbonyl)benzyl]-5-{3-[4-(1,1,1-trifluoro-2-methylpropan-2-yl)phenyl]-1,2,4-oxadiazol-5-yl}pyridin-2(1H)-one

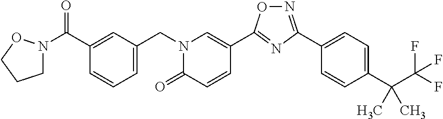 [SMILES: CC(C)(c1ccc(-c2noc(-c3ccc(=O)n(Cc4cccc(C(=O)N5CCCO5)c4)c3)n2)cc1)C(F)(F)F]

Analogously to the process described in Example 22, 95 mg (0.190 mmol) of the compound from Example 24A and 21 mg (0.285 mmol) of isoxazolidine gave 84 mg (82% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.03 (dd, 1H), 7.82-7.76 (m, 2H), 7.63 (d, 2H), 7.51 (d, 1H), 7.45 (t, 1H), 6.75 (d, 1H), 5.28 (s, 2H), 3.97 (t, 2H), 3.90 (t, 2H), 2.35 (quint, 2H), 1.62 (s, 6H).

LC/MS (Method 3, ESIpos): Rₜ=1.19 min; m/z=539 [M+H]⁺.

### Example 32

### 1-{3-[(4-Hydroxypiperidin-1-yl)carbonyl]benzyl}-5-{3-[4-(trifluoromethoxy)phenyl]-1,2,4-oxadiazol-5-yl}pyridin-2(1H)-one

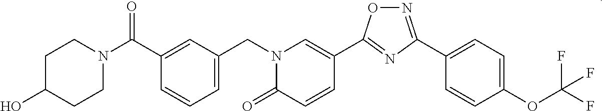 [SMILES: O=C(c1cccc(Cn2cc(-c3nc(-c4ccc(OC(F)(F)F)cc4)no3)ccc2=O)c1)N1CCC(O)CC1]

Analogously to the process described in Example 22, 119 mg (0.250 mmol) of the compound from Example 22A and 38 mg (0.375 mmol) of 4-hydroxypiperidine gave 109 mg (80% of theory) of the title compound. Here, the mobile phase used for chromatographic purification was dichloromethane/methanol 25:1.

¹H NMR (400 MHz, CDCl₃, δ/ppm): 8.38 (d, 1H), 8.16 (d, 2H), 8.03 (dd, 1H), 7.43 (m, 3H), 7.34 (m, 3H), 6.75 (d, 1H), 5.26 (s, 2H), 4.18 (br. s, 1H), 3.98 (m, 1H), 3.64 (br. s, 1H), 3.40 (br. s, 1H), 3.19 (br. s, 1H), 1.97 (br. s, 1H), 1.82 (br. s, 1H), 1.68 (d, 1H), 1.50 (br. s, 1H).

LC/MS (Method 7, ESIpos): Rₜ=1.03 min; m/z=541 [M+H]⁺.

### Example 33

### 1-{3-[(4-Hydroxypiperidin-1-yl)carbonyl]benzyl}-5-{3-[4-(1,1,1-trifluoro-2-methylpropan-2-yl)phenyl]-1,2,4-oxadiazol-5-yl}pyridin-2(1H)-one

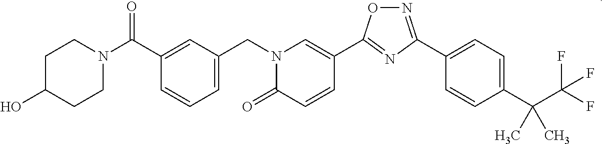 [SMILES: CC(C)(c1ccc(-c2noc(-c3ccc(=O)n(Cc4cccc(C(=O)N5CCC(O)CC5)c4)c3)n2)cc1)C(F)(F)F]

Analogously to the process described in Example 22, 95 mg (0.190 mmol) of the compound from Example 24A and 29 mg (0.285 mmol) of 4-hydroxypiperidine gave 78 mg (72% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.04 (dd, 1H), 7.63 (d, 2H), 7.45-7.42 (m, 3H), 7.40-7.35 (m, 1H), 6.75 (d, 1H), 5.26 (s, 2H), 4.18 (br. s, 1H), 4.02-3.93 (m, 1H), 3.64 (br. s, 1H), 3.39 (br. s, 1H), 3.20 (br. s, 1H), 1.98 (br. s, 1H), 1.82 (br. s, 1H), 1.62 (s, 6H), 1.49 (br. s, 1H).

LC/MS (Method 3, ESIpos): Rₜ=1.09 min; m/z=567 [M+H]⁺.

### Example 34

### 1-[3-(Morpholin-4-ylcarbonyl)benzyl]-5-{3-[4-(trifluoromethoxy)phenyl]-1,2,4-oxadiazol-5-yl}pyridin-2(1H)-one

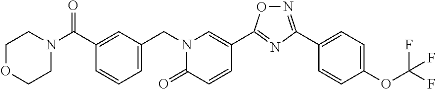 [SMILES: O=C(c1cccc(Cn2cc(-c3nc(-c4ccc(OC(F)(F)F)cc4)no3)ccc2=O)c1)N1CCOCC1]

Analogously to the process described under Example 22, 119 mg (0.250 mmol) of the compound from Example 22A and 33 μl (0.375 mmol) of morpholine gave 105 mg (80% of theory) of the title compound. Here, the mobile phase used for chromatographic purification was dichloromethane/methanol 25:1.

¹H NMR (400 MHz, CDCl₃, δ/ppm): 8.39 (d, 1H), 8.16 (d, 2H), 8.04 (dd, 1H), 7.46-7.43 (m, 3H), 7.41-7.32 (m, 3H), 6.75 (d, 1H), 5.26 (s, 2H), 3.85-3.36 (br. m, 8H).

LC/MS (Method 7, ESIpos): Rₜ=1.11 min; m/z=527 [M+H]⁺.

### Example 35

### 1-[3-(Morpholin-4-ylcarbonyl)benzyl]-5-{3-[4-(1,1,1-trifluoro-2-methylpropan-2-yl)phenyl]-1,2,4-oxadiazol-5-yl}pyridin-2(1H)-one

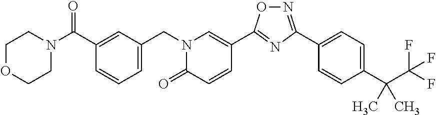 [SMILES: CC(C)(c1ccc(-c2noc(-c3ccc(=O)n(Cc4cccc(C(=O)N5CCOCC5)c4)c3)n2)cc1)C(F)(F)F]

Analogously to the process described under Example 22, 95 mg (0.190 mmol) of the compound from Example 24A and 25 μl (0.285 mmol) of morpholine gave 90 mg (85% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.05 (dd, 1H), 7.63 (d, 2H), 7.45-7.43 (m, 3H), 7.41-7.35 (m, 1H), 6.75 (d, 1H), 5.27 (s, 2H), 3.77 (br. m, 8H), 1.63 (s, 6H).

LC/MS (Method 3, ESIpos): Rₜ=1.16 min; m/z=553 [M+H]⁺.

### Example 36

### 1-{3-[(4-Methylpiperazin-1-yl)carbonyl]benzyl}-5-{3-[4-(trifluoromethoxy)phenyl]-1,2,4-oxadiazol-5-yl}pyridin-2(1H)-one

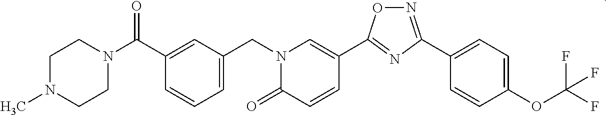 [SMILES: CN1CCN(C(=O)c2cccc(Cn3cc(-c4nc(-c5ccc(OC(F)(F)F)cc5)no4)ccc3=O)c2)CC1]

Analogously to the process described in Example 22, 119 mg (0.250 mmol) of the compound from Example 22A and 38 mg (0.375 mmol) of 1-methylpiperazine gave 101 mg (75% of theory) of the title compound. Here, the mobile phase used for chromatographic purification was dichloromethane/methanol 25:1.

¹H NMR (400 MHz, CDCl₃, δ/ppm): 8.39 (d, 1H), 8.16 (d, 2H), 8.03 (dd, 1H), 7.43 (m, 3H), 7.36 (m, 3H), 6.75 (d, 1H), 5.27 (s, 2H), 3.78 (br. s, 2H), 3.42 (br. s, 2H), 2.48 (br. s, 2H), 2.33 (br. s, 2H), 2.30 (s, 3H).

LC/MS (Method 7, ESIpos): Rₜ=0.80 min; m/z=540 [M+H]⁺.

### Example 37

### 1-{3-[(4-Methylpiperazin-1-yl)carbonyl]benzyl}-5-{3-[4-(1,1,1-trifluoro-2-methylpropan-2-yl)phenyl]-1,2,4-oxadiazol-5-yl}pyridin-2(1H)-one

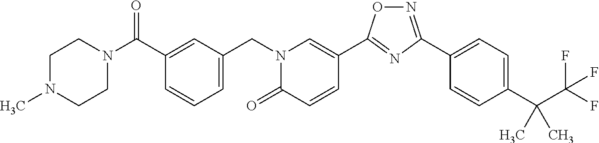 [SMILES: CN1CCN(C(=O)c2cccc(Cn3cc(-c4nc(-c5ccc(C(C)(C)C(F)(F)F)cc5)no4)ccc3=O)c2)CC1]

Analogously to the process described under Example 22, 64 mg (60% of theory) of the title compound were obtained from 95 mg (0.190 mmol) of the compound from Example 24A and 32 μl (0.285 mmol) of 1-methylpiperazine. Here, the mobile phase used for chromatographic purification was dichloromethane/methanol 40:1. The product thus obtained was, after chromatography, triturated twice with in each case 1 ml of pentane and dried under high vacuum to remove residual solvent.

¹H NMR (400 MHz, CDCl₃, δ/ppm): 8.39 (d, 1H), 8.09 (d, 2H), 8.04 (dd, 1H), 7.63 (d, 2H), 7.45-7.41 (m, 3H), 7.40-7.34 (m, 1H), 6.75 (d, 1H), 5.26 (s, 2H), 3.79 (br. s, 2H), 3.42 (br. s, 2H), 2.48 (br. s, 2H), 2.34 (br. s, 2H), 2.31 (s, 3H), 1.62 (s, 6H).

LC/MS (Method 3, ESIpos): Rₜ=0.87 min; m/z=567 [M+H]⁺.

### Example 38

### 1-{3-[(4-Cyclopropylpiperazin-1-yl)carbonyl]benzyl}-5-{3-[4-(trifluoromethoxy)phenyl]-1,2,4-oxadiazol-5-yl}pyridin-2(1H)-one

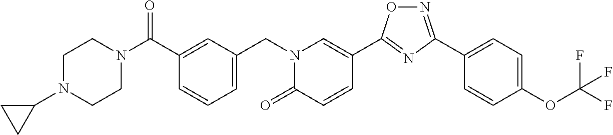 [SMILES: O=C(c1cccc(Cn2cc(-c3nc(-c4ccc(OC(F)(F)F)cc4)no3)ccc2=O)c1)N1CCN(C2CC2)CC1]

Analogously to the process described in Example 22, 95 mg (0.200 mmol) of the compound from Example 22A and 58 mg (0.300 mmol) of 1-cyclopropylpiperazine dihydrochloride gave 90 mg (79% of theory) of the title compound.

¹H NMR (400 MHz, CDCl₃, δ/ppm): 8.39 (d, 1H), 8.15 (d, 2H), 8.03 (dd, 1H), 7.45-7.41 (m, 3H), 7.36-7.32 (m, 1H), 7.34 (d, 2H), 6.75 (d, 1H), 5.27 (s, 2H), 3.73 (br. s, 2H), 3.35 (br. s, 2H), 2.68 (br. s, 2H), 2.53 (br. s, 2H), 1.64-1.60 (m, 1H), 0.49-0.44 (m, 2H), 0.43-0.37 (m, 2H).

LC/MS (Method 7, ESIpos): Rₜ=0.87 min; m/z=566 [M+H]⁺.

### Example 39

### 1-{3-[(4-Cyclopropylpiperazin-1-yl)carbonyl]benzyl}-5-{3-[4-(1,1,1-trifluoro-2-methylpropan-2-yl)phenyl]-1,2,4-oxadiazol-5-yl}pyridin-2(1H)-one

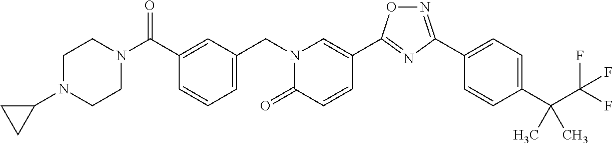 [SMILES: CC(C)(c1ccc(-c2noc(-c3ccc(=O)n(Cc4cccc(C(=O)N5CCN(C6CC6)CC5)c4)c3)n2)cc1)C(F)(F)F]

Analogously to the process described in Example 22, 93 mg (0.185 mmol) of the compound from Example 24A and 55 mg (0.278 mmol) of 1-cyclopropylpiperazine dihydrochloride gave 79 mg (72% of theory) of the title compound. Here, the mobile phase used for chromatographic purification was dichloromethane/methanol 40:1.

¹H NMR (400 MHz, CDCl₃, δ/ppm): 8.39 (d, 1H), 8.09 (d, 2H), 8.05 (dd, 1H), 7.63 (d, 2H), 7.45-7.41 (m, 3H), 7.40-7.35 (m, 1H), 6.75 (d, 1H), 5.27 (s, 2H), 3.73 (br. s, 2H), 3.36 (br. s, 2H), 2.68 (br. s, 2H), 2.54 (br. s, 2H), 1.64-1.60 (m, 1H, obscured), 1.62 (s, 6H), 0.50-0.43 (m, 2H), 0.43-0.38 (m, 2H).

LC/MS (Method 7, ESIpos): Rₜ=0.91 min; m/z=592 [M+H]⁺.

### Example 40

### 1-(3-{[2-Oxo-5-{3-[4-(trifluoromethoxy)phenyl]-1,2,4-oxadiazol-5-yl}pyridin-1(2H)-yl]methyl}phenyl)cyclopropyl acetate

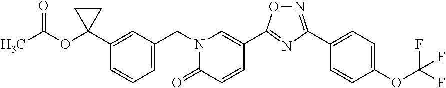 [SMILES: CC(=O)OC1(c2cccc(Cn3cc(-c4nc(-c5ccc(OC(F)(F)F)cc5)no4)ccc3=O)c2)CC1]

Under argon and with ice cooling, 48 mg (1.21 mmol, 60% in mineral oil) of sodium hydride were added to a mixture of 300 mg (0.928 mmol) of the compound from Example 10A in 6 ml of DMF. After 15 min of stirring at RT, 275 mg (1.02 mmol) of the compound from Example 1A dissolved in 1 ml of DMF were added, and the reaction mixture was stirred at RT for 2 h. Subsequently, 40 ml each of water and ethyl acetate were added slowly, and the phases were separated. The aqueous phase was extracted once with 40 ml of ethyl acetate, and the combined organic phases were washed once with 50 ml of saturated sodium chloride solution, dried over sodium sulphate, filtered and concentrated. The residue was triturated with cyclohexane/ethyl acetate 7:3. The solid was filtered off and washed with 4 ml of cyclohexane/ethyl acetate mixture. Drying under high vacuum gave 222 mg (47% of theory) of the title compound.

¹H-NMR (400 MHz, DMSO-d₆, δ/ppm): 9.05-8.98 (m, 1H), 8.19 (d, 2H), 8.07 (dd, 2H), 7.60 (d, 2H), 7.30 (t, 1H), 7.24 (s, 1H), 7.19 (d, 1H), 7.09 (d, 1H), 6.66 (d, 1H), 5.27 (s, 2H), 2.03 (s, 3H), 1.31-1.26 (m, 2H), 1.25-1.19 (m, 2H).

LC/MS (Method 3, ESIpos): Rₜ=1.27 min; m/z=512 [M+H]⁺.

### Example 41

### 1-[3-(2-Hydroxy-2-methylpropyl)benzyl]-5-{3-[4-(trifluoromethoxy)phenyl]-1,2,4-oxadiazol-5-yl}pyridin-2(1H)-one

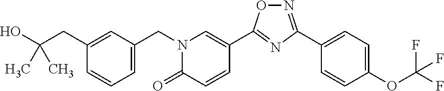 [SMILES: CC(C)(O)Cc1cccc(Cn2cc(-c3nc(-c4ccc(OC(F)(F)F)cc4)no3)ccc2=O)c1]

Under argon and with ice cooling, 52 mg (1.30 mmol, 60% in mineral oil) of sodium hydride were added to a mixture of 323 mg (1.00 mmol) of the compound from Example 10A in 2.5 ml of DMF. After 15 min of stirring at RT, 284 mg (1.10 mmol) of the compound from Example 2A dissolved in 1 ml of DMF were added, and the reaction mixture was stirred at RT for 2 h. Subsequently, 30 ml each of water and ethyl acetate were added slowly, and the phases were separated. The aqueous phase was extracted once with 30 ml of ethyl acetate, and the combined organic phases were washed once with 50 ml of saturated sodium chloride solution, dried over sodium sulphate, filtered and concentrated. The residue was purified by column chromatography (Biotage, silica gel, mobile phase cyclohexane/ethyl acetate 1:1). Drying under high vacuum gave 396 mg (79% of theory) of the title compound.

¹H NMR (400 MHz, CDCl₃, δ/ppm): 8.35 (d, 1H), 8.14 (d, 2H), 8.01 (dd, 1H), 7.36-7.31 (m, 3H), 7.26-7.20 (m, 3H), 6.74 (d, 1H), 5.24 (s, 2H), 2.78 (s, 2H), 1.39 (br. s, 1H), 1.22 (s, 6H).

LC/MS (Method 3, ESIpos): Rₜ=1.23 min; m/z=486 [M+H]⁺.

### Example 42

### 1-[3-(2-Hydroxy-2-methylpropyl)benzyl]-5-{3-[4-(1,1,1-trifluoro-2-methylpropan-2-yl)phenyl]-1,2,4-oxadiazol-5-yl}pyridin-2(1H)-one

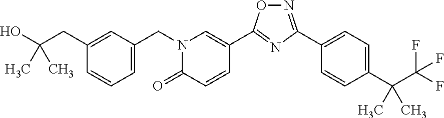 [SMILES: CC(C)(O)Cc1cccc(Cn2cc(-c3nc(-c4ccc(C(C)(C)C(F)(F)F)cc4)no3)ccc2=O)c1]

Analogously to the process described in Example 41, 349 mg (1.00 mmol) of the compound from Example 12A and 284 mg (1.10 mmol) of the compound from Example 2A gave 348 mg (65% of theory) of the title compound.

¹H NMR (400 MHz, CDCl₃, δ/ppm): 8.35 (d, 1H), 8.08 (d, 2H), 8.02 (dd, 1H), 7.63 (d, 2H), 7.36-7.31 (m, 1H), 7.26-7.19 (m, 3H), 6.74 (d, 1H), 5.24 (s, 2H), 2.78 (s, 2H), 1.64 (d, 6H), 1.41 (s, 1H), 1.22 (s, 6H).

LC/MS (Method 3, ESIpos): Rₜ=1.28 min; m/z=512 [M+H]⁺.

### Example 43

### 1-{3-[1-(Hydroxymethyl)cyclopropyl]benzyl}-5-{3-[4-(trifluoromethoxy)phenyl]-1,2,4-oxadiazol-5-yl}pyridin-2(1H)-one

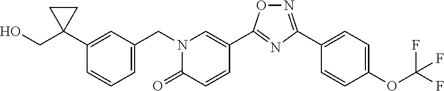 [SMILES: O=c1ccc(-c2nc(-c3ccc(OC(F)(F)F)cc3)no2)cn1Cc1cccc(C2(CO)CC2)c1]

At RT, 656 μl (0.656 mmol) of a 1 M solution of tetra-n-butylammonium fluoride (TBAF) in THF were added to a solution of 350 mg (0.547 mmol) of the compound from Example 25A in 5 ml of THF. The mixture was stirred at RT for 2 h. 30 ml of ethyl acetate were then added, and the mixture was washed once with 30 ml of water. The aqueous phase was reextracted once with 30 ml of ethyl acetate. The combined organic phases were washed once with 60 ml of saturated sodium chloride solution, filtered and concentrated. The residue was triturated with 8 ml of cyclohexane/ethyl acetate. The solid was filtered off and washed twice with in each case 2 ml of cyclohexane/ethyl acetate (1:1). Drying under high vacuum gave, as a first batch, 106 mg (40% of theory) of the title compound. The filtrate obtained when filtering off the solid was concentrated and the residue was purified by column chromatography (Biotage, silica gel, mobile phase cyclohexane/ethyl acetate 1:1). Drying under high vacuum gave, as a second batch, a further 116 mg (42% of theory) of the title compound. A total of 222 mg (82% of theory) of the title compound was thus obtained.

¹H NMR (400 MHz, CDCl₃, δ/ppm): 8.37 (d, 1H), 8.15 (d, 2H), 8.02 (dd, 1H), 7.42 (s, 1H), 7.37-7.31 (m, 4H), 7.23-7.18 (m, 1H), 6.75 (d, 1H), 5.23 (s, 2H), 3.69 (d, 2H), 1.55 (t, 1H), 0.89 (d, 4H).

LC/MS (Method 1, ESIpos): Rₜ=1.42 min; m/z=484 [M+H]⁺.

### Example 44

### 1-{3-[1-(Hydroxymethyl)cyclopropyl]benzyl}-5-{3-[4-(1,1,1-trifluoro-2-methylpropan-2-yl)phenyl]-1,2,4-oxadiazol-5-yl}pyridin-2(1H)-one

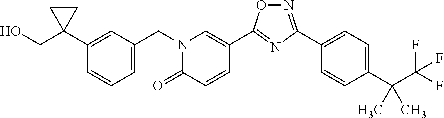 [SMILES: CC(C)(c1ccc(-c2noc(-c3ccc(=O)n(Cc4cccc(C5(CO)CC5)c4)c3)n2)cc1)C(F)(F)F]

At RT, 339 μl (0.339 mmol) of a 1 M solution of tetra-n-butylammonium fluoride (TBAF) in THF were added to a solution of 188 mg (0.283 mmol) of the compound from Example 26A in 2 ml of THF. The mixture was stirred at RT for 1 h. 30 ml of ethyl acetate were then added, and the mixture was washed once with 30 ml of water. The aqueous phase was reextracted once with 30 ml of ethyl acetate. The combined organic phases were washed once with 60 ml of saturated sodium chloride solution, filtered and concentrated. The residue was purified by column chromatography (Biotage, silica gel, mobile phase cyclohexane/ethyl acetate 7:3). Drying under high vacuum gave 100 mg (69% of theory) of the title compound.

¹H NMR (400 MHz, CDCl₃, δ/ppm): 8.36 (d, 1H), 8.08 (d, 2H), 8.03 (dd, 1H), 7.63 (d, 2H), 7.42 (s, 1H), 7.38-7.30 (m, 2H), 7.23-7.18 (m, 1H), 6.74 (d, 1H), 5.23 (s, 2H), 3.69 (d, 2H), 1.62 (s, 6H), 1.55 (t, 1H), 0.89 (d, 4H).

LC/MS (Method 3, ESIpos): Rₜ=1.26 min; m/z=510 [M+H]⁺.

### Example 45

### 1-(4-{[2-oxo-5-{3-[4-(trifluoromethoxy)phenyl]-1,2,4-oxadiazol-5-yl}pyridin-1(2H)-yl]methyl}pyridin-2-yl)piperidine-4-carbonitrile

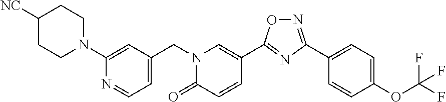 [SMILES: N#CC1CCN(c2cc(Cn3cc(-c4nc(-c5ccc(OC(F)(F)F)cc5)no4)ccc3=O)ccn2)CC1]

A mixture of 106 mg (0.237 mmol) of the compound from Example 27A and 522 mg (4.74 mmol) of 4-cyanopiperidine was heated in a microwave oven (Biotage Initiator, with dynamic irradiation power control) at 160° C. for 3 h. After cooling to RT, a little methanol was added and the mixture was purified directly by preparative HPLC (Method 13). The product fractions were combined and concentrated to a residual volume of aqueous phase. The pH was adjusted to 8-9 by addition of saturated aqueous sodium bicarbonate solution, and the mixture was extracted twice with 50 ml of ethyl acetate each time. The combined organic phases were dried over sodium sulphate, filtered and concentrated. Drying under high vacuum gave 87 mg (66% of theory) of the title compound.

¹H NMR (400 MHz, CDCl₃, δ/ppm): 8.34 (d, 1H), 8.19-8.12 (m, 3H), 8.06 (dd, 1H), 7.35 (d, 2H), 6.77 (d, 1H), 6.62 (s, 1H), 6.55 (d, 1H), 5.14 (s, 2H), 3.84 (ddd, 2H), 3.49 (ddd, 2H), 2.88 (tt, 1H), 2.04-1.96 (m, 2H), 1.96-1.86 (m, 2H).

LC/MS (Method 3, ESIpos): Rₜ=1.01 min; m/z=523 [M+H]⁺.

### Example 46

### 1-(4-{[2-oxo-5-{3-[4-(1,1,1-trifluoro-2-methylpropan-2-yl)phenyl]-1,2,4-oxadiazol-5-yl}pyridin-1(2H)-yl]methyl}pyridin-2-yl)piperidine-4-carbonitrile

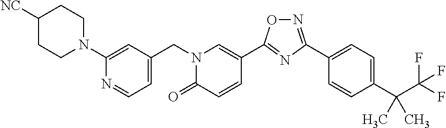 [SMILES: CC(C)(c1ccc(-c2noc(-c3ccc(=O)n(Cc4ccnc(N5CCC(C#N)CC5)c4)c3)n2)cc1)C(F)(F)F]

Analogously to the process described in Example 45, 113 mg (0.238 mmol) of the compound from Example 29A and 525 mg (4.76 mmol) of 4-cyanopiperidine gave 76 mg (57% of theory) of the title compound.

¹H NMR (400 MHz, CDCl₃, δ/ppm): 8.34 (d, 1H), 8.17 (d, 1H), 8.11-8.04 (m, 3H), 7.63 (d, 2H), 6.77 (d, 1H), 6.62 (s, 1H), 6.55 (d, 1H), 5.14 (s, 2H), 3.84 (ddd, 2H), 3.49 (ddd, 2H), 2.87 (tt, 1H), 2.04-1.96 (m, 2H), 1.96-1.86 (m, 2H), 1.63 (s, 6H).

LC/MS (Method 1, ESIpos): Rₜ=1.31 min; m/z=549 [M+H]⁺.

### Example 47

### 1-{[2-(4-Methylpiperazin-1-yl)pyridin-4-yl]methyl}-5-(3-{4-[(trifluoromethyl)sulphanyl]phenyl}-1,2,4-oxadiazol-5-yl)pyridin-2(1H)-one

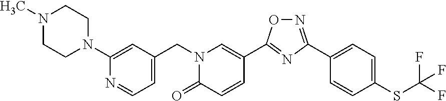 [SMILES: CN1CCN(c2cc(Cn3cc(-c4nc(-c5ccc(SC(F)(F)F)cc5)no4)ccc3=O)ccn2)CC1]

At 0° C., a solution of 16 μl (0.253 mmol) of iodomethane in 2 ml of dichloromethane was added dropwise to a solution of 130 mg (0.253 mmol) of the compound from Example 32A in 3 ml of dichloromethane. After the reaction mixture had been stirred at RT for about 16 h, it was diluted with about 5 ml of dichloromethane and washed successively with about 10 ml each of water and saturated sodium chloride solution. After drying over anhydrous magnesium sulphate, the mixture was filtered and the filtrate was concentrated to dryness on a rotary evaporator. The crude product was purified by MPLC (silica gel, mobile phase dichloromethane/methanol 10:1). The combined product fractions were once more concentrated to dryness and the residue was then triturated with a mixture of 4 ml of pentane and 1 ml of diisopropyl ether. Filtration and drying of the solid under high vacuum gave 33 mg (23% of theory) of the title compound.

¹H NMR (400 MHz, CDCl₃, δ/ppm): 8.34 (d, 1H), 8.17 (d, 1H), 8.16 (d, 2H), 8.05 (dd, 1H), 7.79 (d, 2H), 6.77 (d, 1H), 6.60 (s, 1H), 6.52 (d, 1H), 5.14 (s, 2H), 3.59-3.56 (m, 4H), 2.53-2.49 (m, 4H), 2.34 (s, 3H).

LC/MS (Method 3, ESIpos): Rₜ=0.89 min; m/z=529 [M+H]⁺.

### Example 48

### 1-{[2-(4-Methylpiperazin-1-yl)pyridin-4-yl]methyl}-5-(3-{4-[1-(trifluoromethyl)cyclopropyl]phenyl}-1,2,4-oxadiazol-5-yl)pyridin-2(1H)-one

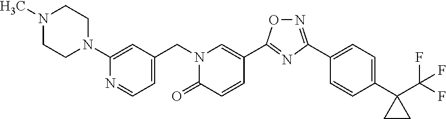 [SMILES: CN1CCN(c2cc(Cn3cc(-c4nc(-c5ccc(C6(C(F)(F)F)CC6)cc5)no4)ccc3=O)ccn2)CC1]

Analogously to the process described under Example 47, two batches of in each case 100 mg (0.191 mmol) of the compound from Example 34A and 13 μl (0.211 mmol) of iodomethane gave 29 mg (13% of theory, 90% purity) of the title compound. Here, trituration after chromatographic purification, as described above, was dispensed with.

¹H NMR (400 MHz, CDCl₃, δ/ppm): 8.34 (d, 1H), 8.17 (d, 1H), 8.08 (d, 2H), 8.05 (dd, 1H), 7.59 (d, 2H), 6.76 (d, 1H), 6.60 (s, 1H), 6.52 (d, 1H), 5.14 (s, 2H), 3.59-3.56 (m, 4H), 2.53-2.49 (m, 4H), 2.34 (s, 3H), 1.44-1.40 (m, 2H), 1.10-1.16 (m, 2H).

LC/MS (Method 3, ESIpos): Rₜ=0.85 min; m/z=523 [M+H]⁺.

### Example 49

### 1-({2-[4-(2,2,2-Trifluoroethyl)piperazin-1-yl]pyridin-4-yl}methyl)-5-{3-[4-(trifluoromethoxy)phenyl]-1,2,4-oxadiazol-5-yl}pyridin-2(1H)-one

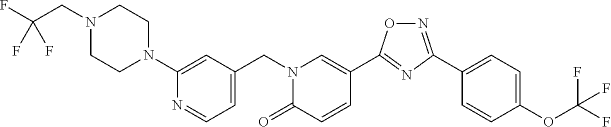 [SMILES: O=c1ccc(-c2nc(-c3ccc(OC(F)(F)F)cc3)no2)cn1Cc1ccnc(N2CCN(CC(F)(F)F)CC2)c1]

At 0° C. and under argon, 131 μl (0.938 mmol) of triethylamine and 127 μl (0.750 mmol) of trifluoromethanesulphonic anhydride were added to a solution of 55 μl (0.750 mmol) of 2,2,2-trifluoroethanol in 5 ml of dichloromethane. The mixture was stirred at 0° C. for 2 h. 187 mg (0.375 mmol) of the compound from Example 31A dissolved in 2 ml of DMF were then added, and the mixture was stirred at RT for 5 days. 25 ml each of water and dichloromethane were then added to the mixture, and the phases were separated. The aqueous phase was extracted twice with in each case 30 ml of dichloromethane, and the combined organic phases were dried over sodium sulphate, filtered and concentrated. The residue was purified by column chromatography (Biotage, silica gel, mobile phase cyclohexane/ethyl acetate 7:3). Drying under high vacuum gave 53 mg (24% of theory) of the title compound.

¹H NMR (400 MHz, CDCl₃, δ/ppm): 8.33 (d, 1H), 8.19-8.13 (m, 3H), 8.05 (dd, 1H), 7.34 (d, 2H), 6.77 (d, 1H), 6.58 (s, 1H), 6.53 (d, 1H), 5.14 (s, 2H), 3.60-3.55 (m, 4H), 3.02 (q, 2H), 2.79-2.77 (m, 4H).

LC/MS (Method 3, ESIpos): Rₜ=1.18 min; m/z=581 [M+H]⁺.

### Example 50

### 1-({2-[4-(2,2,2-Trifluoroethyl)piperazin-1-yl]pyridin-4-yl}methyl)-5-{3-[4-(1,1,1-trifluoro-2-methylpropan-2-yl)phenyl]-1,2,4-oxadiazol-5-yl}pyridin-2(1H)-one

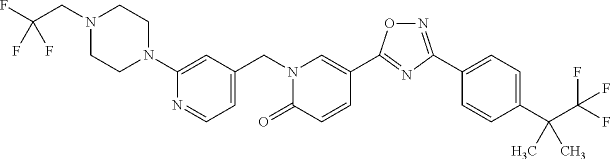 [SMILES: CC(C)(c1ccc(-c2noc(-c3ccc(=O)n(Cc4ccnc(N5CCN(CC(F)(F)F)CC5)c4)c3)n2)cc1)C(F)(F)F]

Analogously to the process described in Example 49, 37 μl (0.515 mmol) of 2,2,2-trifluoroethanol, 90 μl (0.643 mmol) of triethylamine, 87 μl (0.515 mmol) of trifluoromethanesulphonic anhydride and 135 mg (0.257 mmol) of the compound from Example 33A gave 39 mg (25% of theory) of the title compound. In this case, the reaction time was 8 days at RT; the mobile phase used for the chromatographic purification was cyclohexane/ethyl acetate 1:1.

¹H NMR (400 MHz, CDCl₃, δ/ppm): 8.34 (d, 1H), 8.17 (d, 1H), 8.11-8.04 (m, 3H), 7.63 (d, 2H), 6.76 (d, 1H), 6.58 (s, 1H), 6.53 (d, 1H), 5.14 (s, 2H), 3.60-3.55 (m, 4H), 3.02 (q, 2H), 2.79-2.74 (m, 4H), 1.62 (s, 6H).

LC/MS (Method 3, ESIpos): Rₜ=1.19 min; m/z=607 [M+H]⁺.

### Example 51

### 1-{[2-(4-Cyclopropylpiperazin-1-yl)pyridin-4-yl]methyl}-5-{3-[4-(trifluoromethoxy)phenyl]-1,2,4-oxadiazol-5-yl}pyridin-2(1H)-one

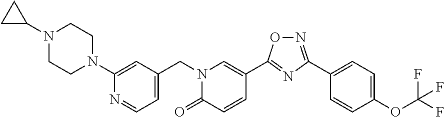 [SMILES: O=c1ccc(-c2nc(-c3ccc(OC(F)(F)F)cc3)no2)cn1Cc1ccnc(N2CCN(C3CC3)CC2)c1]

Under argon, 228 μl (3.98 mmol) of acetic acid, 417 mg (2.39 mmol) of [(1-ethoxycyclopropyl)oxy](trimethyl)silane and some molecular sieve (3 Å) were added to a mixture of 168 mg (0.297 mmol, purity 75%) of the compound from Example 31A in 4 ml of methanol. After 10 min of stirring at RT, 75 mg (1.19 mmol) of sodium cyanoborohydride were added, and the mixture was stirred at a bath temperature of 70° C. for 2 h. After cooling to RT, the molecular sieve was filtered off and washed with methanol, and the filtrate was concentrated. The residue was purified by preparative HPLC (Method 13). The combined product fractions were concentrated to a residual volume of aqueous phase and adjusted to a pH of 8 with saturated aqueous sodium bicarbonate solution. The mixture was then extracted twice with ethyl acetate, and the combined organic phases were dried over sodium sulphate, filtered and concentrated. Drying under high vacuum gave 52 mg (24% of theory) of the title compound.

¹H NMR (400 MHz, CDCl₃, δ/ppm): 8.33 (d, 1H), 8.18-8.13 (m, 3H), 8.05 (dd, 1H), 7.34 (d, 2H), 6.76 (d, 1H), 6.58 (s, 1H), 6.50 (d, 1H), 5.14 (s, 2H), 3.55-3.49 (m, 4H), 2.74-2.68 (m, 4H), 1.29-1.22 (m, 1H), 0.51-0.44 (m, 4H).

LC/MS (Method 3, ESIpos): Rₜ=0.93 min; m/z=539 [M+H]⁺.

### Example 52

### 1-{[2-(4-Cyclopropylpiperazin-1-yl)pyridin-4-yl]methyl}-5-(3-{4-[(trifluoromethyl)sulphanyl]phenyl}-1,2,4-oxadiazol-5-yl)pyridin-2(1H)-one

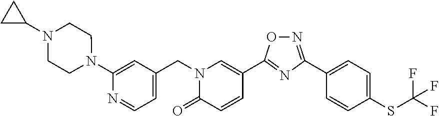 [SMILES: O=c1ccc(-c2nc(-c3ccc(SC(F)(F)F)cc3)no2)cn1Cc1ccnc(N2CCN(C3CC3)CC2)c1]

Under argon, 305 mg (1.75 mmol) of [(1-ethoxycyclopropyl)oxy](trimethyl)silane and some molecular sieve (3 Å) were added to a solution of 150 mg (0.292 mmol) of the compound from Example 32A in 4 ml of methanol and 167 μl (2.92 mmol) of acetic acid. After 10 min of stirring at RT, 55 mg (0.875 mmol) of sodium cyanoborohydride were added, and the mixture was stirred at a bath temperature of 70° C. for 2 h. After cooling to RT, the molecular sieve was filtered off and washed with methanol, and the filtrate was freed from all volatible components on a rotary evaporator. The residue obtained was dissolved in about 30 ml of ethyl acetate and the mixture was extracted successively with about 30 ml each of saturated sodium bicarbonate solution and saturated sodium chloride solution. After drying over anhydrous magnesium sulphate, the organic phase was re-concentrated and the crude product was purified by preparative HPLC (Method 17). The combined product fractions were freed from the solvent and then treated with pentane in an ultrasonic bath for 10 min. Filtration and drying of the solid under high vacuum thus gave 15 mg (9% of theory) of the title compound.

¹H NMR (400 MHz, CDCl₃, δ/ppm): 8.34 (d, 1H), 8.17 (d, 1H), 8.16 (d, 2H), 8.05 (dd, 1H), 7.78 (d, 2H), 6.77 (d, 1H), 6.59 (s, 1H), 6.51 (d, 1H), 5.14 (s, 2H), 3.57-3.53 (m, 4H), 2.77-2.75 (m, 4H), 1.75-1.67 (m, 1H), 0.56-0.48 (m, 4H).

LC/MS (Method 3, ESIpos): Rₜ=0.99 min; m/z=555 [M+H]⁺.

### Example 53

### 1-{[2-(4-Cyclopropylpiperazin-1-yl)pyridin-4-yl]methyl}-5-{3-[4-(1,1,1-trifluoro-2-methylpropan-2-yl)phenyl]-1,2,4-oxadiazol-5-yl}pyridin-2(1H)-one

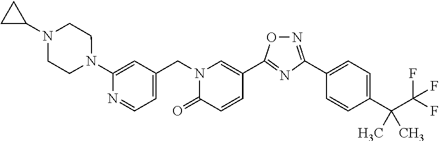 [SMILES: CC(C)(c1ccc(-c2noc(-c3ccc(=O)n(Cc4ccnc(N5CCN(C6CC6)CC5)c4)c3)n2)cc1)C(F)(F)F]

Analogously to the process described under Example 51, 25 mg (16% of theory) of the title compound were obtained from 145 mg (0.276 mmol) of the compound from Example 33A and 333 μl (1.659 mmol) of [(1-ethoxycyclopropyl)oxy](trimethyl)silane.

¹H NMR (400 MHz, CDCl₃, δ/ppm): 8.34 (d, 1H), 8.17 (d, 1H), 8.11-8.04 (m, 3H), 7.63 (d, 2H), 6.76 (d, 1H), 6.59 (s, 1H), 6.51 (d, 1H), 5.14 (s, 2H), 3.55-3.50 (m, 4H), 2.75-2.69 (m, 4H), 1.62 (s, 6H), 1.28-1.23 (m, 1H), 0.51-0.46 (m, 4H).

LC/MS (Method 3, ESIpos): Rₜ=1.01 min; m/z=565 [M+H]⁺.

### Example 54

### 1-{[2-(4-Cyclopropylpiperazin-1-yl)pyridin-4-yl]methyl}-5-(3-{4-[1-(trifluoromethyl)cyclopropyl]phenyl}-1,2,4-oxadiazol-5-yl)pyridin-2(1H)-one

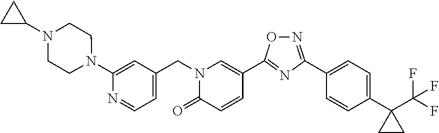 [SMILES: O=c1ccc(-c2nc(-c3ccc(C4(C(F)(F)F)CC4)cc3)no2)cn1Cc1ccnc(N2CCN(C3CC3)CC2)c1]

Analogously to the process described in Example 52, 200 mg (0.383 mmol) of the compound from Example 34A and 400 mg (2.30 mmol) of [(1-ethoxycyclopropyl)oxy](trimethyl)silane gave 73 mg (34% of theory) of the title compound. Here, the final treatment with pentane in an ultrasonic bath was dispensed with.

¹H NMR (400 MHz, CDCl₃, δ/ppm): 8.33 (d, 1H), 8.17 (d, 1H), 8.08 (d, 2H), 8.05 (dd, 1H), 7.59 (d, 2H), 6.76 (d, 1H), 6.58 (s, 1H), 6.50 (d, 1H), 5.14 (s, 2H), 3.55-3.51 (m, 4H), 2.73-2.69 (m, 4H), 1.66-1.63 (m, 1H, partially superimposed by the water signal), 1.43-1.40 (m, 2H), 1.10-1.06 (m, 2H), 0.50-0.46 (m, 4H).

LC/MS (Method 3, ESIpos): Rₜ=0.93 min; m/z=563 [M+H]⁺.

### Example 55

### 1-{[2-(4-Acetylpiperazin-1-yl)pyridin-4-yl]methyl}-5-(3-{4-[(trifluoromethyl)sulphanyl]phenyl}-1,2,4-oxadiazol-5-yl)pyridin-2(1H)-one

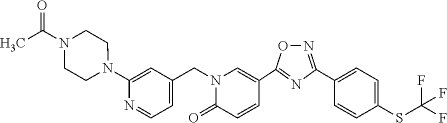 [SMILES: CC(=O)N1CCN(c2cc(Cn3cc(-c4nc(-c5ccc(SC(F)(F)F)cc5)no4)ccc3=O)ccn2)CC1]

At a temperature of 0° C., a solution of 11 μl (0.150 mmol) of acetyl chloride in 2 ml of dichloromethane was added dropwise to a solution of 70 mg (0.136 mmol) of the compound from Example 32A and 23 μl (0.163 mmol) of triethylamine in 3 ml of dichloromethane. After the reaction mixture had been stirred at RT for 1 h, 20 ml of water were added. The resulting precipitate was filtered off with suction, washed with a little cold water and dried under high vacuum. This gave 62 mg (80% of theory) of the title compound.

¹H NMR (400 MHz, CDCl₃, δ/ppm): 8.35 (d, 1H), 8.19 (d, 1H), 8.16 (d, 2H), 8.06 (dd, 1H), 7.79 (d, 2H), 6.78 (d, 1H), 6.60 (s, 1H), 6.57 (d, 1H), 5.16 (s, 2H), 3.75-3.73 (m, 2H), 3.66-3.63 (m, 2H), 3.60-3.56 (m, 2H), 3.54-3.50 (m, 2H), 2.14 (s, 3H).

LC/MS (Method 7, ESIpos): Rₜ=1.01 min; m/z=557 [M+H]⁺.

### Example 56

### 1-{[2-(4-Acetylpiperazin-1-yl)pyridin-4-yl]methyl}-5-(3-{4-[1-(trifluoromethyl)cyclopropyl]phenyl}-1,2,4-oxadiazol-5-yl)pyridin-2(1H)-one

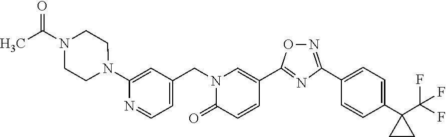 [SMILES: CC(=O)N1CCN(c2cc(Cn3cc(-c4nc(-c5ccc(C6(C(F)(F)F)CC6)cc5)no4)ccc3=O)ccn2)CC1]

At a temperature of 0° C., a solution of 15 μl (0.211 mmol) of acetyl chloride in 2 ml of dichloromethane was added dropwise to a solution of 100 mg (0.191 mmol) of the compound from Example 34A and 32 μl (0.230 mmol) of triethylamine in 3 ml of dichloromethane. After the reaction mixture had been stirred at RT for 1 h, it was diluted with about 10 ml of dichloromethane and transferred into a separating funnel. The mixture was washed successively with water and saturated sodium chloride solution. The organic phase was dried over anhydrous magnesium sulphate and then filtered, and the solvent was removed on a rotary evaporator. The residue obtained was purified by preparative HPLC (Method 17). This gave 81 mg (75% of theory) of the title compound.

¹H NMR (400 MHz, CDCl₃, δ/ppm): 8.34 (d, 1H), 8.18 (d, 1H), 8.08 (d, 2H), 8.07 (dd, 1H), 7.60 (d, 2H), 6.77 (d, 1H), 6.60 (s, 1H), 6.57 (d, 1H), 5.15 (s, 2H), 3.75-3.73 (m, 2H), 3.66-3.62 (m, 2H), 3.60-3.56 (m, 2H), 3.54-3.51 (m, 2H), 2.14 (s, 3H), 1.44-1.40 (m, 2H), 1.10-1.06 (m, 2H).

LC/MS (Method 7, ESIpos): Rₜ=1.00 min; m/z=565 [M+H]⁺.

### Example 57

### 1-[(6-Chloropyridin-3-yl)methyl]-5-{3-[4-(trifluoromethoxy)phenyl]-1,2,4-oxadiazol-5-yl}pyridin-2(1H)-one

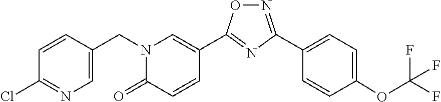 [SMILES: O=c1ccc(-c2nc(-c3ccc(OC(F)(F)F)cc3)no2)cn1Cc1ccc(Cl)nc1]

Analogously to the process described in Example 41, 250 mg (0.773 mmol) of the compound from Example 10A and 163 mg (1.01 mmol) of 2-chloro-5-(chloromethyl)pyridine gave 222 mg (64% of theory) of the title compound. Here, the alkylation step was carried out not at RT but at a temperature of 40° C.; the reaction time for this partial step was 30 min.

¹H NMR (400 MHz, CDCl₃, δ/ppm): 8.49 (d, 1H), 8.40 (d, 1H), 8.16 (d, 2H), 8.04 (dd, 1H), 7.77 (dd, 1H), 7.38-7.32 (m, 3H), 6.75 (d, 1H), 5.22 (s, 2H).

LC/MS (Method 3, ESIpos): Rₜ=1.20 min; m/z=449 [M+H]⁺.

### Example 58

### 1-[(6-Chloropyridin-3-yl)methyl]-5-{3-[4-(1,1,1-trifluoro-2-methylpropan-2-yl)phenyl]-1,2,4-oxadiazol-5-yl}pyridin-2(1H)-one

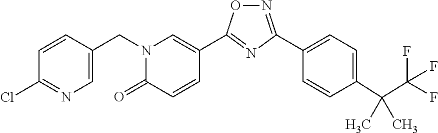 [SMILES: CC(C)(c1ccc(-c2noc(-c3ccc(=O)n(Cc4ccc(Cl)nc4)c3)n2)cc1)C(F)(F)F]

Analogously to the process described in Example 41, 349 mg (1.00 mmol) of the compound from Example 12A and 211 mg (1.30 mmol) of 2-chloro-5-(chloromethyl)pyridine gave 308 mg (65% of theory) of the title compound. Here, the chromatographic purification was carried out using the mobile phase cyclohexane/ethyl acetate 2:1.

¹H-NMR (400 MHz, DMSO-d₆, δ/ppm): 9.13 (d, 1H), 8.52 (d, 1H), 8.10-8.06 (m, 3H), 7.89 (dd, 1H), 7.78 (d, 2H), 7.52 (d, 1H), 6.64 (d, 1H), 5.30 (s, 2H), 1.61 (s, 6H).

LC/MS (Method 3, ESIpos): Rₜ=1.26 min; m/z=475 [M+H]⁺.

### Example 59

### 1-{[6-(Methylamino)pyridin-3-yl]methyl}-5-{3-[4-(trifluoromethoxy)phenyl]-1,2,4-oxadiazol-5-yl}pyridin-2(1H)-one

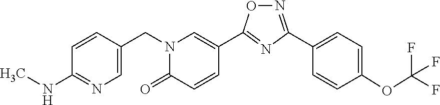 [SMILES: CNc1ccc(Cn2cc(-c3nc(-c4ccc(OC(F)(F)F)cc4)no3)ccc2=O)cn1]

A mixture of 195 mg (0.435 mmol) of the compound from Example 57 and 5.4 ml (43.5 mmol) of a 33% strength solution of methylamine in ethanol was stirred in a microwave oven (Biotage Initiator, with dynamic irradiation power control) at 150° C. for 5 h. After cooling to RT, the volatile constituents were removed on a rotary evaporator. The residue was taken up in an acetonitrile/water mixture and purified by preparative HPLC (Method 14). The combined product fractions were concentrated to a residual volume of aqueous phase and adjusted to pH 8 with saturated aqueous sodium bicarbonate solution. The mixture was then extracted twice with in each case 50 ml of ethyl acetate, and the combined organic phases were dried over sodium sulphate, filtered and concentrated. Drying under high vacuum gave 181 mg (89% of theory) of the title compound.

¹H NMR (400 MHz, CDCl₃, δ/ppm): 8.35 (d, 1H), 8.20-8.12 (m, 3H), 8.02-7.96 (m, 1H), 7.58-7.51 (m, 1H), 7.34 (d, 2H), 6.71 (d, 1H), 6.40 (d, 1H), 5.09 (s, 2H), 4.73-4.62 (m, 1H), 2.93 (d, 3H).

LC/MS (Method 3, ESIpos): Rₜ=0.83 min; m/z=444 [M+H]⁺.

### Example 60

### 1-{[6-(Methylamino)pyridin-3-yl]methyl}-5-{3-[4-(1,1,1-trifluoro-2-methylpropan-2-yl)phenyl]-1,2,4-oxadiazol-5-yl}pyridin-2(1H)-one

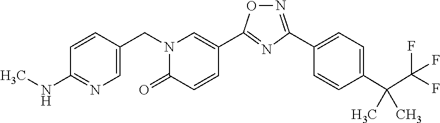 [SMILES: CNc1ccc(Cn2cc(-c3nc(-c4ccc(C(C)(C)C(F)(F)F)cc4)no3)ccc2=O)cn1]

A mixture of 100 mg (0.211 mmol) of the compound from Example 58 and 2.6 ml (21.1 mmol) of a 33% strength solution of methylamine in ethanol was stirred in a microwave oven (Biotage Initiator, with dynamic irradiation power control) at 150° C. for 5 h. After cooling to RT, the volatile components were removed on a rotary evaporator and the residue was stirred in 3 ml of acetonitrile. The solid formed was filtered off and washed twice with 0.5 ml of acetonitrile. Drying under high vacuum gave, as a first batch, 31 mg (31% of theory) of the title compound. The filtrate obtained in the filtration of the solid was combined with the wash solutions and concentrated. The residue was crystallized from 2 ml of methanol and filtered off. Washing with a little methanol and drying under high vacuum gave, as a second batch, a further 31 mg (32% of theory) of the title compound. A total of 62 mg (63% of theory) of the title compound was thus obtained.

¹H NMR (400 MHz, CDCl₃, δ/ppm): 8.35 (d, 1H), 8.18 (d, 1H), 8.09 (d, 2H), 8.00 (dd, 1H), 7.63 (d, 2H), 7.55 (dd, 1H), 6.71 (d, 1H), 6.40 (d, 1H), 5.09 (s, 2H), 4.67 (q, 1H), 2.93 (d, 3H), 1.63 (s, 6H).

LC/MS (Method 3, ESIpos): Rₜ=0.97 min; m/z=470 [M+H]⁺.

### Example 61

### 1-{[6-(Ethylamino)pyridin-3-yl]methyl}-5-{3-[4-(trifluoromethoxy)phenyl]-1,2,4-oxadiazol-5-yl}pyridin-2(1H)-one

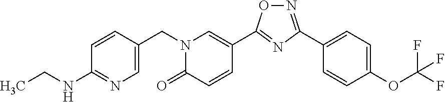 [SMILES: CCNc1ccc(Cn2cc(-c3nc(-c4ccc(OC(F)(F)F)cc4)no3)ccc2=O)cn1]

A mixture of 100 mg (0.223 mmol) of the compound from Example 57 and 1.8 ml (22.3 mmol) of a 70% strength solution of ethylamine in water was stirred in a microwave oven (Biotage Initiator, with dynamic irradiation power control) at 150° C. for 45 min. A further 1 ml of the 70% strength solution of ethylamine in water was then added, and the mixture was stirred in a microwave oven at 150° C. for another 2 h. Subsequently, another 1.5 ml of the 70% strength solution of ethylamine in water were added, and the mixture was stirred in a microwave oven at 150° C. for a further 3 h. After cooling to RT, the volatile constituents were removed on a rotary evaporator. The residue was taken up in 8 ml of methanol and purified by preparative HPLC (Method 13). The combined product fractions were concentrated to a residual volume of aqueous phase and adjusted to pH 8-9 with saturated aqueous sodium bicarbonate solution. The mixture was then extracted twice with 30 ml of ethyl acetate, and the combined organic phases were dried over sodium sulphate, filtered and concentrated. Drying under high vacuum gave 50 mg (49% of theory) of the title compound.

¹H NMR (400 MHz, CDCl₃, δ/ppm): 8.35 (d, 1H), 8.18-8.13 (m, 3H), 7.99 (dd, 1H), 7.53 (dd, 1H), 7.34 (d, 2H), 6.72 (d, 1H), 6.38 (d, 1H), 5.08 (s, 2H), 4.65 (br. s, 1H), 3.31 (quint, 2H), 1.25 (t, 3H).

LC/MS (Method 1, ESIpos): Rₜ=1.12 min; m/z=458 [M+H]⁺.

### Example 62

### 1-{[6-(Ethylamino)pyridin-3-yl]methyl}-5-{3-[4-(1,1,1-trifluoro-2-methylpropan-2-yl)phenyl]-1,2,4-oxadiazol-5-yl}pyridin-2(1H)-one

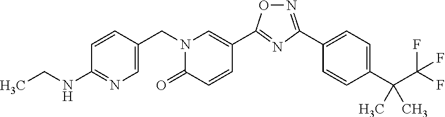 [SMILES: CCNc1ccc(Cn2cc(-c3nc(-c4ccc(C(C)(C)C(F)(F)F)cc4)no3)ccc2=O)cn1]

Analogously to the process described in Example 59, 100 mg (0.211 mmol) of the compound from Example 58 and 1.7 ml (21.1 mmol) of a 70% strength solution of ethylamine in water gave 52 mg (51% of theory) of the title compound. Here, purification by preparative HPLC was carried out according to Method 13.

¹H NMR (400 MHz, CDCl₃, δ/ppm): 8.35 (d, 1H), 8.16 (d, 1H), 8.09 (d, 2H), 8.00 (dd, 1H), 7.63 (d, 2H), 7.54 (dd, 1H), 6.71 (d, 1H), 6.38 (d, 1H), 5.08 (s, 2H), 4.76 (br. s, 1H), 3.31 (m, 2H), 1.63 (s, 6H), 1.25 (t, 3H).

LC/MS (Method 3, ESIpos): Rₜ=0.92 min; m/z=484 [M+H]⁺.

### Example 63

### 1-({6-[(3-Hydroxypropyl)amino]pyridin-3-yl}methyl)-5-{3-[4-(trifluoromethoxy)phenyl]-1,2,4-oxadiazol-5-yl}pyridin-2(1H)-one

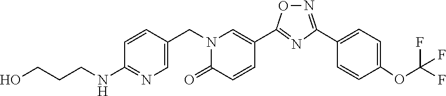 [SMILES: O=c1ccc(-c2nc(-c3ccc(OC(F)(F)F)cc3)no2)cn1Cc1ccc(NCCCO)nc1]

A mixture of 200 mg (0.445 mmol) of the compound from Example 57 and 682 μl (8.91 mmol) of 3-amino-1-propanol in 0.5 ml of diethylene glycol dimethyl ether (diglyme) was stirred in a microwave oven (Biotage Initiator, with dynamic irradiation power control) at 180° C. for 3 h. After cooling to RT, the reaction mixture was introduced into 50 ml of water and extracted three times with 30 ml of ethyl acetate each time. The combined organic phases were dried over sodium sulphate, filtered and concentrated. The residue was triturated with about 20 ml of hot ethyl acetate. The solid formed was filtered off, washed twice with in each case 2 ml of ethyl acetate and dried under high vacuum. This gave 140 mg (65% of theory) of the title compound.

¹H NMR (400 MHz, CDCl₃, δ/ppm): 8.35 (d, 1H), 8.16 (d, 2H), 8.12 (s, 1H), 8.00 (dd, 1H), 7.51 (d, 1H), 7.35 (d, 2H), 6.72 (d, 1H), 6.40 (d, 1H), 5.07 (s, 2H), 4.77-4.70 (m, 1H), 4.23 (br. s, 1H), 3.67-3.60 (m, 2H), 3.58-3.52 (m, 2H), 1.79-1.72 (m, 2H).

LC/MS (Method 3, ESIpos): Rₜ=0.80 min; m/z=488 [M+H]⁺.

### Example 64

### 1-({6-[(3-Hydroxypropyl)amino]pyridin-3-yl}methyl)-5-{3-[4-(1,1,1-trifluoro-2-methylpropan-2-yl)phenyl]-1,2,4-oxadiazol-5-yl}pyridin-2(1H)-one

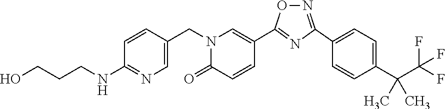 [SMILES: CC(C)(c1ccc(-c2noc(-c3ccc(=O)n(Cc4ccc(NCCCO)nc4)c3)n2)cc1)C(F)(F)F]

A mixture of 100 mg (0.211 mmol) of the compound from Example 58 and 322 μl (4.21 mmol) of 3-amino-1-propanol in 0.5 ml of diethylene glycol dimethyl ether (diglyme) was stirred in a microwave oven (Biotage Initiator, with dynamic irradiation power control) at 180° C. for 3 h. After cooling to RT, the volatile constituents were removed on a rotary evaporator. The residue was taken up in acetonitrile and purified by preparative HPLC (method 13). The combined product fractions were concentrated to a residual volume of aqueous phase and adjusted to pH 8-9 with saturated aqueous sodium bicarbonate solution. The mixture was then extracted twice with 30 ml of ethyl acetate, and the combined organic phases were dried over sodium sulphate, filtered and concentrated. Drying under high vacuum gave 17 mg (16% of theory) of the title compound.

¹H NMR (400 MHz, CDCl₃, δ/ppm): 8.37 (d, 1H), 8.12-8.08 (m, 3H), 8.01 (dd, 1H), 7.63 (d, 2H), 7.55 (dd, 1H), 6.72 (d, 1H), 6.44 (d, 1H), 5.33-5.17 (m, 1H), 5.07 (s, 2H), 3.67-3.63 (m, 2H), 3.57-3.51 (m, 2H), 1.77 (dt, 2H), 1.63 (s, 6H).

LC/MS (Method 3, ESIpos): Rₜ=0.88 min; m/z=514 [M+H]⁺.

### Example 65

### 2-(4-Methylbenzyl)-6-{3-[4-(trifluoromethoxy)phenyl]-1,2,4-oxadiazol-5-yl}pyridazin-3(2H)-one

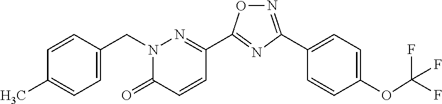 [SMILES: Cc1ccc(Cn2nc(-c3nc(-c4ccc(OC(F)(F)F)cc4)no3)ccc2=O)cc1]

41 mg (0.369 mmol) of potassium tert-butoxide were added to a mixture of 100 mg (0.284 mmol, purity 92%) of the compound from Example 14A and 68 mg (0.369 mmol) of 1-(bromomethyl)-4-methylbenzene in 4 ml of THF. The reaction mixture was stirred first at RT for 15 min and then at 50° C. for 1.5 h. A further 94 mg (0.846 mmol) of potassium tert-butoxide were then added and, at 50° C., a few millilitres of DMF such that the solid components dissolved and the reaction, initially slow, was accelerated. After 15 min, the mixture was allowed to cool to RT, and ethyl acetate and water were added. After phase separation, the aqueous phase was extracted twice with ethyl acetate, and the combined organic phases were washed once with saturated sodium chloride solution, dried over magnesium sulphate, filtered and concentrated. The residue was purified by two preparative HPLCs according to Method 15. Drying under high vacuum gave 32 mg (26% of theory) of the title compound.

¹H NMR (400 MHz, CDCl₃, δ/ppm): 8.20 (d, 2H), 8.01 (d, 1H), 7.43 (d, 2H), 7.36 (d, 2H), 7.16 (d, 2H), 7.07 (d, 1H), 5.43 (s, 2H), 2.33 (s, 3H).

LC/MS (Method 3, ESIpos): Rₜ=1.36 min; m/z=429 [M+H]⁺.

### Example 66

### 1-(3-{[6-oxo-3-{3-[4-(trifluoromethoxy)phenyl]-1,2,4-oxadiazol-5-yl}pyridazin-1(6H)-yl]methyl}phenyl)piperidine-4-carbonitrile

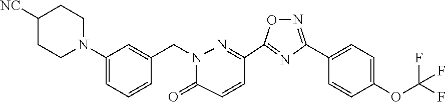 [SMILES: N#CC1CCN(c2cccc(Cn3nc(-c4nc(-c5ccc(OC(F)(F)F)cc5)no4)ccc3=O)c2)CC1]

Under argon, a mixture of 250 mg (0.507 mmol) of the compound from Example 35A, 67 mg (0.608 mmol) of 4-cyanopiperidine, 23 mg (0.101 mmol) of palladium(II) acetate, 30 mg (0.101 mmol) of 2-(di-tert-butylphosphino)biphenyl and 68 mg (0.710 mmol) of sodium tert-butoxide in 5 ml of toluene was stirred in a microwave oven (Biotage Initiator, with dynamic irradiation power control) at 120° C. for 3 h. After cooling to RT, a further 67 mg (0.608 mmol) of 4-cyanopiperidine, dissolved in 1 ml of DMF, were added, and the mixture was stirred at 150° C. in the microwave oven for another 1 h. After cooling to RT, 30 ml each of ethyl acetate and water were added and the mixture was transferred into a separating funnel and extracted. After phase separation, the aqueous phase was re-extracted twice with 50 ml of ethyl acetate, and the combined organic phases were washed once with saturated sodium chloride solution, dried over sodium sulphate, filtered and concentrated. The residue was purified by column chromatography (Biotage, silica gel, mobile phase cyclohexane/ethyl acetate 7:3). Drying under high vacuum gave 11 mg (4% of theory, purity 95%) of the title compound.

¹H NMR (400 MHz, CDCl₃, δ/ppm): 8.20 (d, 2H), 8.03 (d, 1H), 7.37 (d, 2H), 7.26-7.22 (m, 1H), 7.14 (s, 1H), 7.08 (d, 1H), 7.05 (d, 1H), 6.88 (dd, 1H), 5.41 (s, 2H), 3.48-3.38 (m, 2H), 3.16-3.08 (m, 2H), 2.79 (tt, 1H), 2.11-1.93 (m, 4H).

LC/MS (Method 3, ESIpos): Rₜ=1.29 min; m/z=523 [M+H]⁺.

### Example 67

tert-Butyl 4-(3-{[6-oxo-3-{3-[4-(trifluoromethoxy)phenyl]-1,2,4-oxadiazol-5-yl}pyridazin-1(6H)-yl]methyl}phenyl)piperazine-1-carboxylate

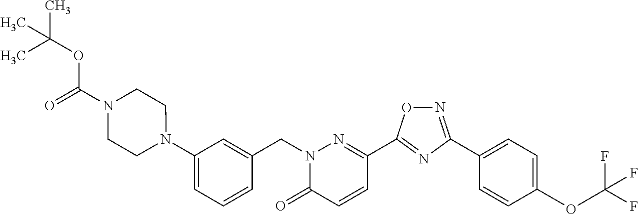 [SMILES: CC(C)(C)OC(=O)N1CCN(c2cccc(Cn3nc(-c4nc(-c5ccc(OC(F)(F)F)cc5)no4)ccc3=O)c2)CC1]

Under argon, a mixture of 250 mg (0.507 mmol) of the compound from Example 35A, 113 mg (0.608 mmol) of tert-butyl piperazine-1-carboxylate, 23 mg (0.101 mmol) of palladium(II) acetate, 30 mg (0.101 mmol) of 2-(di-tert-butylphosphino)biphenyl and 68 mg (0.710 mmol) of sodium tert-butoxide in 5 ml of toluene was stirred in a microwave oven (Biotage Initiator, with dynamic irradiation power control) at 120° C. for 1 h. After cooling to RT, the mixture was filtered and the filtrate was purified directly by column chromatography (Biotage, silica gel, mobile phase cyclohexane/ethyl acetate 7:3). After concentration of the combined product fractions, the residue was triturated with hot methanol and the solid formed was washed twice with in each case 0.5 ml of methanol. Drying under high vacuum gave 24 mg (7% of theory, purity 90%) of the title compound.

¹H NMR (400 MHz, CDCl₃, δ/ppm): 8.20 (d, 2H), 8.02 (d, 1H), 7.37 (d, 2H), 7.27-7.22 (m, 1H), 7.13 (s, 1H), 7.08 (d, 1H), 7.04 (d, 1H), 6.87 (dd, 1H), 5.42 (s, 2H), 3.59-3.54 (m, 4H), 3.17-3.12 (m, 4H), 1.48 (s, 9H).

LC/MS (Method 1, ESIpos): Rₜ=1.66 min; m/z=599 [M+H]⁺.

### Example 68

### Methyl 3-{[6-oxo-3-{3-[4-(trifluoromethoxy)phenyl]-1,2,4-oxadiazol-5-yl}pyridazin-1(6H)-yl]methyl}benzoate

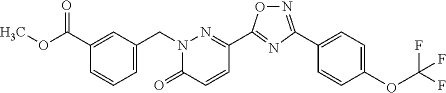 [SMILES: COC(=O)c1cccc(Cn2nc(-c3nc(-c4ccc(OC(F)(F)F)cc4)no3)ccc2=O)c1]

Analogously to the process described in Example 41, 1.33 g (4.10 mmol) of the compound from Example 14A and 1.03 g (4.51 mmol) of methyl 3-bromomethylbenzoate gave 1.30 g (67% of theory) of the title compound. In this case, the reaction time in the alkylation step was 1 h, and purification by column chromatography was carried out using the following method: Interchim, silica gel, mobile phase cyclohexane/ethyl acetate 7:3.

¹H-NMR (400 MHz, DMSO-d₆, δ/ppm): 8.22 (d, 2H), 8.16 (d, 1H), 8.00 (s, 1H), 7.92 (d, 1H), 7.66 (d, 1H), 7.62 (d, 2H), 7.54 (t, 1H), 7.26 (d, 1H), 5.50 (s, 2H), 3.85 (s, 3H).

LC/MS (Method 3, ESIpos): Rₜ=1.28 min; m/z=473 [M+H]⁺.

### Example 69

### Methyl 3-{[6-oxo-3-{3-[4-(1,1,1-trifluoro-2-methylpropan-2-yl)phenyl]-1,2,4-oxadiazol-5-yl}pyridazin-1(6H)-yl]methyl}benzoate

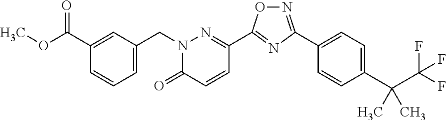 [SMILES: COC(=O)c1cccc(Cn2nc(-c3nc(-c4ccc(C(C)(C)C(F)(F)F)cc4)no3)ccc2=O)c1]

Analogously to the process described in Example 41, 1.43 g (4.09 mmol) of the compound from Example 15A and 1.03 g (4.50 mmol) of methyl 3-bromomethylbenzoate gave 1.65 g (81% of theory) of the title compound. In this case, the reaction time in the alkylation step was 1 h, and purification by column chromatography was carried out using the following method: Büchi, silica gel, mobile phase cyclohexane/ethyl acetate 7:3.

¹H-NMR (400 MHz, DMSO-d₆, δ/ppm): 8.16 (d, 1H), 8.11 (d, 2H), 8.00 (s, 1H), 7.92 (d, 1H), 7.79 (d, 2H), 7.66 (d, 1H), 7.54 (t, 1H), 7.25 (d, 1H), 5.50 (s, 2H), 1.61 (s, 6H).

LC/MS (Method 3, ESIpos): Rₜ=1.33 min; m/z=499 [M+H]⁺.

### Example 70

### N-Methyl-3-{[6-oxo-3-{3-[4-(1,1,1-trifluoro-2-methylpropan-2-yl)phenyl]-1,2,4-oxadiazol-5-yl}pyridazin-1(6H)-yl]methyl}benzamide

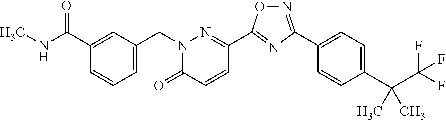 [SMILES: CNC(=O)c1cccc(Cn2nc(-c3nc(-c4ccc(C(C)(C)C(F)(F)F)cc4)no3)ccc2=O)c1]

75 mg (0.150 mmol) of the compound from Example 39A, dissolved in 1 ml of dichloromethane, were added to a solution of 375 μl (0.750 mmol) of a 2 M solution of methylamine in THF. The reaction mixture was stirred at RT for 1 h. The mixture was then filtered through a thin layer of silica gel, the filter cake was rinsed with dichloromethane and the filtrate was concentrated on a rotary evaporator. The residue was purified by thick-layer chromatography (silica gel, 20×20 cm, mobile phase dichloromethane/methanol 95:5). The product-containing zone was extracted with dichloromethane/methanol 95:5. Concentration and drying of the residue under high vacuum gave 70 mg (91% of theory, purity 97%) of the title compound.

¹H NMR (400 MHz, CDCl₃, δ/ppm): 8.13 (d, 2H), 8.06 (d, 1H), 7.88 (s, 1H), 7.76 (d, 1H), 7.69-7.62 (m, 3H), 7.43 (t, 1H), 7.09 (d, 1H), 6.24 (br. s, 1H), 5.49 (s, 2H), 3.01 (d, 3H).

LC/MS (Method 7, ESIpos): Rₜ=1.15 min; m/z=498 [M+H]⁺.

### Example 71

### N-Isopropyl-3-{[6-oxo-3-{3-[4-(1,1,1-trifluoro-2-methylpropan-2-yl)phenyl]-1,2,4-oxadiazol-5-yl}pyridazin-1(6H)-yl]methyl}benzamide

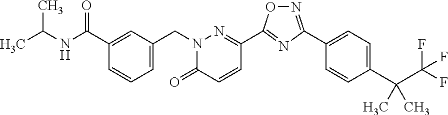 [SMILES: CC(C)NC(=O)c1cccc(Cn2nc(-c3nc(-c4ccc(C(C)(C)C(F)(F)F)cc4)no3)ccc2=O)c1]

75 mg (0.150 mmol) of the compound from Example 39A, dissolved in 1 ml of dichloromethane, were added to a solution of 19 μl (0.225 mmol) of isopropylamine and 52 μl (0.300 mmol) of N,N-diisopropylethylamine in 1.5 ml of dichloromethane. The reaction mixture was stirred at RT for 1 h. The mixture was then filtered through a thin layer of silica gel, the filter cake was rinsed with dichloromethane and the filtrate was concentrated on a rotary evaporator. The residue was purified by thick-layer chromatography (silica gel, 20×20 cm, mobile phase dichloromethane/methanol 100:3). The product-containing zone was extracted with dichloromethane/methanol 95:5. Concentration and drying of the residue under high vacuum gave 71 mg (90% of theory, purity 99%) of the title compound.

¹H NMR (400 MHz, CDCl₃, δ/ppm): 8.06 (d, 2H), 8.00 (d, 1H), 7.83 (s, 1H), 7.67 (d, 1H), 7.60-7.54 (m, 3H), 7.35 (t, 1H), 7.03 (d, 1H), 6.19 (d, 1H), 5.44 (s, 2H), 4.27-4.13 (m, 1H), 1.56 (s, 6H), 1.19 (d, 6H).

LC/MS (Method 7, ESIpos): Rₜ=1.26 min; m/z=526 [M+H]⁺.

### Example 72

### N-Benzyl-3-{[6-oxo-3-{3-[4-(1,1,1-trifluoro-2-methylpropan-2-yl)phenyl]-1,2,4-oxadiazol-5-yl}pyridazin-1(6H)-yl]methyl}benzamide

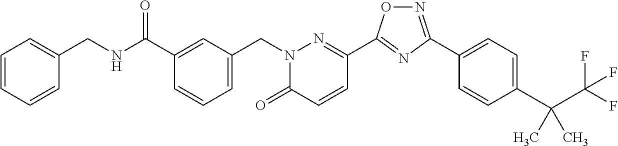 [SMILES: CC(C)(c1ccc(-c2noc(-c3ccc(=O)n(Cc4cccc(C(=O)NCc5ccccc5)c4)n3)n2)cc1)C(F)(F)F]

Analogously to the process described under Example 71, 75 mg (0.150 mmol) of the compound from Example 39A and 25 μl (0.225 mmol) of benzylamine gave 86 mg (97% of theory, 96% pure) of the title compound.

¹H NMR (400 MHz, CDCl₃, δ/ppm): 8.06 (d, 2H), 7.98 (d, 1H), 7.86 (s, 1H), 7.71 (d, 1H), 7.60-7.56 (m, 3H), 7.36 (t, 1H), 7.30-7.25 (m, 3H), 7.23-7.19 (m, 1H), 7.20 (s, 1H), 7.00 (d, 1H), 6.50 (t, broad, 1H), 5.42 (s, 2H), 4.57 (d, 2H), 1.56 (s, 6H).

LC/MS (Method 7, ESIpos): Rₜ=1.32 min; m/z=574 [M+H]⁺.

### Example 73

### N-(3-Hydroxypropyl)-3-{[6-oxo-3-{3-[4-(1,1,1-trifluoro-2-methylpropan-2-yl)phenyl]-1,2,4-oxadiazol-5-yl}pyridazin-1(6H)-yl]methyl}benzamide

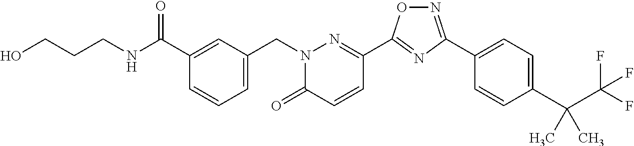 [SMILES: CC(C)(c1ccc(-c2noc(-c3ccc(=O)n(Cc4cccc(C(=O)NCCCO)c4)n3)n2)cc1)C(F)(F)F]

Analogously to the process described under Example 71, 75 mg (0.150 mmol) of the compound from Example 39A and 17 μl (0.225 mmol) of 3-amino-1-propanol gave 75 mg (91% of theory, purity 98%) of the title compound. Here, the mobile phase used for preparative thick-layer chromatography was dichloromethane/methanol 95:5.

¹H NMR (400 MHz, CDCl₃, δ/ppm): 8.13 (d, 2H), 8.06 (d, 1H), 7.91 (s, 1H), 7.79 (d, 1H), 7.70-7.63 (m, 3H), 7.44 (t, 1H), 7.08 (d, 1H), 6.86-6.80 (m, 1H), 5.49 (s, 2H), 3.73 (q, 2H), 3.63 (q, 2H), 3.14 (t, 1H), 1.81 (quint, 2H), 1.63 (s, 6H).

LC/MS (Method 7, ESIpos): Rₜ=1.09 min; m/z=542 [M+H]⁺.

### Example 74

### 2-[3-(Azetidin-1-ylcarbonyl)benzyl]-6-{3-[4-(trifluoromethoxy)phenyl]-1,2,4-oxadiazol-5-yl}pyridazin-3(2H)-one

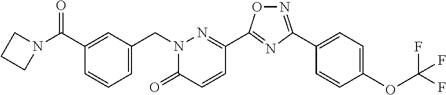 [SMILES: O=C(c1cccc(Cn2nc(-c3nc(-c4ccc(OC(F)(F)F)cc4)no3)ccc2=O)c1)N1CCC1]

Analogously to the process described in Example 71, 80 mg (0.168 mmol) of the compound from Example 37A and 14 mg (0.252 mmol) of azetidine gave 75 mg (84% of theory, purity 93%) of the title compound.

¹H NMR (400 MHz, CDCl₃, δ/ppm): 8.20 (d, 2H), 8.04 (d, 1H), 7.78 (s, 1H), 7.62 (t, 2H), 7.41 (t, 1H), 7.37 (d, 2H), 7.09 (d, 1H), 5.48 (s, 2H), 4.31 (t, 2H), 4.22 (t, 2H), 2.33 (quint, 2H).

LC/MS (Method 3, ESIpos): Rₜ=1.10 min; m/z=498 [M+H]⁺.

### Example 75

### 2-[3-(Azetidin-1-ylcarbonyl)benzyl]-6-{3-[4-(1,1,1-trifluoro-2-methylpropan-2-yl)phenyl]-1,2,4-oxadiazol-5-yl}pyridazin-3(2H)-one

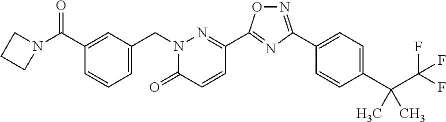 [SMILES: CC(C)(c1ccc(-c2noc(-c3ccc(=O)n(Cc4cccc(C(=O)N5CCC5)c4)n3)n2)cc1)C(F)(F)F]

Analogously to the process described in Example 71, 75 mg (0.150 mmol) of the compound from Example 39A and 13 μl (0.225 mmol) of azetidine gave 81 mg (99% of theory, purity 96%) of the title compound.

¹H NMR (400 MHz, CDCl₃, δ/ppm): 8.07 (d, 2H), 7.99 (d, 1H), 7.71 (s, 1H), 7.61-7.52 (m, 4H), 7.34 (t, 1H), 7.01 (d, 1H), 5.41 (s, 2H), 4.24 (t, 2H), 4.15 (t, 2H), 2.26 (quint, 2H), 1.56 (s, 6H).

LC/MS (Method 7, ESIpos): Rₜ=1.21 min; m/z=524 [M+H]⁺.

### Example 76

### 2-{3-[(3-Hydroxyazetidin-1-yl)carbonyl]benzyl}-6-{3-[4-(trifluoromethoxy)phenyl]-1,2,4-oxadiazol-5-yl}pyridazin-3(2H)-one

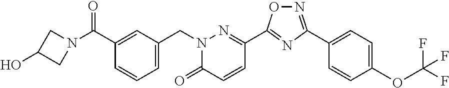 [SMILES: O=C(c1cccc(Cn2nc(-c3nc(-c4ccc(OC(F)(F)F)cc4)no3)ccc2=O)c1)N1CC(O)C1]

Analogously to the process described in Example 71, 80 mg (0.168 mmol) of the compound from Example 37A and 28 mg (0.252 mmol) of 3-hydroxyazetidine hydrochloride gave 56 mg (63% of theory, purity 97%) of the title compound. Here, the mobile phase used for preparative thick-layer chromatography was dichloromethane/methanol 95:5.

¹H NMR (400 MHz, CDCl₃, δ/ppm): 8.20 (d, 2H), 8.04 (d, 1H), 7.78 (s, 1H), 7.62 (dd, 2H), 7.44-7.39 (m, 1H), 7.36 (d, 2H), 7.09 (d, 1H), 5.48 (d, 2H), 4.75-4.65 (m, 1H), 4.50-4.41 (m, 2H), 4.24-4.17 (m, 1H), 4.08-4.02 (m, 1H), 2.68-2.64 (m, 1H).

LC/MS (Method 3, ESIpos): Rₜ=0.99 min; m/z=514 [M+H]⁺.

### Example 77

### 2-{3-[(3-Hydroxyazetidin-1-yl)carbonyl]benzyl}-6-{3-[4-(1,1,1-trifluoro-2-methylpropan-2-yl)phenyl]-1,2,4-oxadiazol-5-yl}pyridazin-3(2H)-one

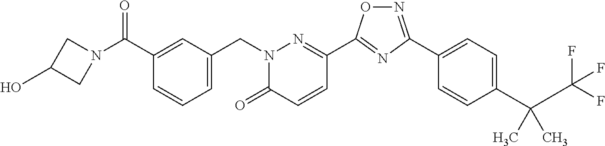 [SMILES: CC(C)(c1ccc(-c2noc(-c3ccc(=O)n(Cc4cccc(C(=O)N5CC(O)C5)c4)n3)n2)cc1)C(F)(F)F]

Analogously to the process described in Example 71, 75 mg (0.150 mmol) of the compound from Example 39A and 25 mg (0.225 mmol) of 3-hydroxyazetidine hydrochloride gave 54 mg (65% of theory, purity 97%) of the title compound. Here, the mobile phase used for preparative thick-layer chromatography was dichloromethane/methanol 95:5; the extraction of the product zone was carried out with dichloromethane/methanol 9:1.

¹H NMR (400 MHz, CDCl₃, δ/ppm): 8.13 (d, 2H), 8.06 (d, 1H), 7.79 (s, 1H), 7.68-7.58 (m, 4H), 7.44-7.39 (m, 1H), 7.09 (d, 1H), 5.48 (d, 2H), 4.74-4.65 (m, 1H), 4.50-4.41 (m, 2H), 4.25-4.16 (m, 1H), 4.10-4.01 (m, 1H), 2.73 (br. s, 1H), 1.63 (s, 6H).

LC/MS (Method 7, ESIpos): Rₜ=1.08 min; m/z=540 [M+H]⁺.

### Example 78

### 2-[3-(Pyrrolidin-1-ylcarbonyl)benzyl]-6-{3-[4-(trifluoromethoxy)phenyl]-1,2,4-oxadiazol-5-yl}pyridazin-3(2H)-one

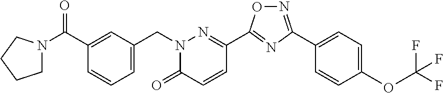 [SMILES: O=C(c1cccc(Cn2nc(-c3nc(-c4ccc(OC(F)(F)F)cc4)no3)ccc2=O)c1)N1CCCC1]

Analogously to the process described in Example 71, 80 mg (0.168 mmol) of the compound from Example 37A and 21 μl (0.252 mmol) of pyrrolidine gave 84 mg (90% of theory, purity 92%) of the title compound.

¹H NMR (400 MHz, CDCl₃, δ/ppm): 8.23-8.17 (m, 2H), 8.04 (d, 1H), 7.68 (s, 1H), 7.58 (d, 1H), 7.49 (d, 1H), 7.41 (d, 1H), 7.37 (d, 2H), 7.09 (d, 1H), 5.48 (s, 2H), 3.63 (t, 2H), 3.42 (t, 2H), 2.00-1.92 (m, 2H), 1.91-1.82 (m, 2H).

LC/MS (Method 3, ESIpos): Rₜ=1.14 min; m/z=512 [M+H]⁺.

### Example 79

### 2-[3-(Pyrrolidin-1-ylcarbonyl)benzyl]-6-{3-[4-(1,1,1-trifluoro-2-methylpropan-2-yl)phenyl]-1,2,4-oxadiazol-5-yl}pyridazin-3(2H)-one

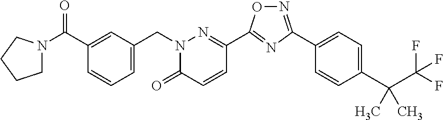 [SMILES: CC(C)(c1ccc(-c2noc(-c3ccc(=O)n(Cc4cccc(C(=O)N5CCCC5)c4)n3)n2)cc1)C(F)(F)F]

75 mg (0.150 mmol) of the compound from Example 39A, dissolved in 0.75 ml of THF, were added to a solution of 18 μl (0.225 mmol) of pyrrolidine and 52 μl (0.300 mmol) of N,N-diisopropylethylamine in 1.5 ml of THF. The reaction mixture was stirred at RT for 2 h. 30 ml of ethyl acetate were then added, and the mixture was washed once with 50 ml of water. The aqueous phase was reextracted once with 30 ml of ethyl acetate. The combined organic phases were dried over sodium sulphate, filtered and concentrated. The residue was purified by column chromatography (Biotage, silica gel, mobile phase dichloromethane/methanol 100:2). Drying under high vacuum gave 47 mg (58% of theory) of the title compound.

¹H NMR (400 MHz, CDCl₃, δ/ppm): 8.14 (d, 2H), 8.05 (d, 1H), 7.69-7.63 (m, 3H), 7.59 (d, 1H), 7.49 (d, 1H), 7.51-7.47 (m, 1H), 7.08 (d, 1H), 5.48 (s, 2H), 3.63 (t, 2H), 3.42 (t, 2H), 2.00-1.91 (m, 2H), 1.90-1.82 (m, 2H), 1.63 (s, 6H).

LC/MS (Method 3, ESIpos): Rₜ=1.20 min; m/z=538 [M+H]⁺.

### Example 80

### 2-[3-(1,2-Oxazolidin-2-ylcarbonyl)benzyl]-6-{3-[4-(trifluoromethoxy)phenyl]-1,2,4-oxadiazol-5-yl}pyridazin-3(2H)-one

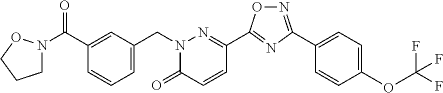 [SMILES: O=C(c1cccc(Cn2nc(-c3nc(-c4ccc(OC(F)(F)F)cc4)no3)ccc2=O)c1)N1CCCO1]

Analogously to the process described in Example 71, 80 mg (0.168 mmol) of the compound from Example 37A and 18 mg (0.252 mmol) of 1,2-oxazolidine gave 59 mg (66% of theory, purity 97%) of the title compound.

¹H NMR (400 MHz, CDCl₃, δ/ppm): 8.20 (d, 2H), 8.04 (d, 1H), 7.94 (s, 1H), 7.75 (d, 1H), 7.64 (d, 1H), 7.41 (t, 1H), 7.36 (d, 2H), 7.09 (d, 1H), 5.50 (s, 2H), 3.98 (t, 2H), 3.89 (t, 2H), 2.35 (quint, 2H).

LC/MS (Method 3, ESIpos): Rₜ=1.11 min; m/z=514 [M+H]⁺.

### Example 81

### 2-[3-(1,2-Oxazolidin-2-ylcarbonyl)benzyl]-6-{3-[4-(1,1,1-trifluoro-2-methylpropan-2-yl)phenyl]-1,2,4-oxadiazol-5-yl}pyridazin-3(2H)-one

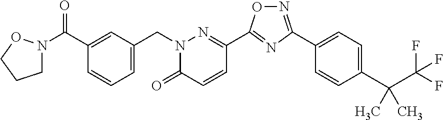 [SMILES: CC(C)(c1ccc(-c2noc(-c3ccc(=O)n(Cc4cccc(C(=O)N5CCCO5)c4)n3)n2)cc1)C(F)(F)F]

Analogously to the process described in Example 71, 75 mg (0.150 mmol) of the compound from Example 39A and 16 mg (0.225 mmol) of 1,2-oxazolidine gave 71 mg (85% of theory, purity 98%) of the title compound.

¹H NMR (400 MHz, CDCl₃, δ/ppm): 8.07 (d, 2H), 7.98 (d, 1H), 7.87 (s, 1H), 7.68 (d, 1H), 7.60-7.56 (m, 3H), 7.34 (t, 1H), 7.01 (d, 1H), 5.43 (s, 2H), 3.91 (t, 2H), 3.82 (t, 2H), 2.28 (quint, 2H), 1.56 (s, 6H).

LC/MS (Method 7, ESIpos): Rₜ=1.21 min; m/z=540 [M+H]⁺.

### Example 82

### 2-{3-[(4-Hydroxypiperidin-1-yl)carbonyl]benzyl}-6-{3-[4-(trifluoromethoxy)phenyl]-1,2,4-oxadiazol-5-yl}pyridazin-3(2H)-one

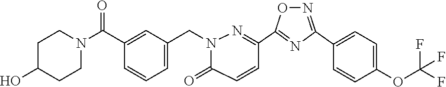 [SMILES: O=C(c1cccc(Cn2nc(-c3nc(-c4ccc(OC(F)(F)F)cc4)no3)ccc2=O)c1)N1CCC(O)CC1]

Analogously to the process described in Example 71, 80 mg (0.168 mmol) of the compound from Example 37A and 25 mg (0.252 mmol) of 4-hydroxypiperidine gave 67 mg (67% of theory, purity 91%) of the title compound. Here, the mobile phase used for preparative thick-layer chromatography was dichloromethane/methanol 95:5; the extraction of the product zone was carried out with dichloromethane/methanol 9:1.

¹H NMR (400 MHz, CDCl₃, δ/ppm): 8.20 (d, 2H), 8.04 (d, 1H), 7.60-7.55 (m, 2H), 7.44-7.34 (m, 4H), 7.09 (d, 1H), 5.48 (s, 2H), 4.20 (br. s, 1H), 4.01-3.92 (m, 1H), 3.65 (br. s, 1H), 3.37 (br. s, 1H), 3.19 (br. s, 1H), 2.05-1.45 (m, 5H).

LC/MS (Method 3, ESIpos): Rₜ=1.00 min; m/z=542 [M+H]⁺.

### Example 83

### 2-{3-[(4-Hydroxypiperidin-1-yl)carbonyl]benzyl}-6-{3-[4-(1,1,1-trifluoro-2-methylpropan-2-yl)phenyl]-1,2,4-oxadiazol-5-yl}pyridazin-3(2H)-one

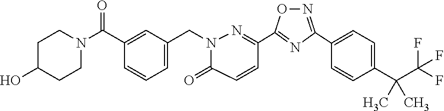 [SMILES: CC(C)(c1ccc(-c2noc(-c3ccc(=O)n(Cc4cccc(C(=O)N5CCC(O)CC5)c4)n3)n2)cc1)C(F)(F)F]

Analogously to the process described in Example 71, 75 mg (0.150 mmol) of the compound from Example 39A and 23 mg (0.225 mmol) of 4-hydroxypiperidine gave 67 mg (76% of theory, purity 97%) of the title compound. Here, the mobile phase used for preparative thick-layer chromatography was dichloromethane/methanol 95:5.

¹H NMR (400 MHz, CDCl₃, δ/ppm): 8.13 (d, 2H), 8.06 (d, 1H), 7.65 (d, 2H), 7.61-7.55 (m, 2H), 7.47-7.33 (m, 2H), 7.08 (d, 1H), 5.48 (s, 2H), 4.20 (br. s, 1H), 3.96 (br. s, 1H), 3.65 (br. s, 1H), 3.36 (br. s, 1H), 3.19 (br. s, 1H), 1.98 (br. s, 1H), 1.81 (br. s, 1H), 1.63 (s, 6H), 1.60-1.44 (m, 2H, obscured).

LC/MS (Method 7, ESIpos): Rₜ=1.10 min; m/z=568 [M+H]⁺.

### Example 84

### 2-[3-(Morpholin-4-ylcarbonyl)benzyl]-6-{3-[4-(trifluoromethoxy)phenyl]-1,2,4-oxadiazol-5-yl}pyridazin-3(2H)-one

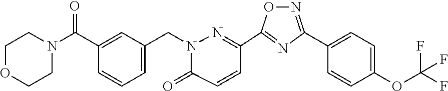 [SMILES: O=C(c1cccc(Cn2nc(-c3nc(-c4ccc(OC(F)(F)F)cc4)no3)ccc2=O)c1)N1CCOCC1]

Analogously to the process described in Example 71, 80 mg (0.168 mmol) of the compound from Example 37A and 22 mg (0.252 mmol) of morpholine gave 89 mg (95% of theory, purity 95%) of the title compound.

¹H NMR (400 MHz, CDCl₃, δ/ppm): 8.20 (d, 2H), 8.05 (d, 1H), 7.62-7.55 (m, 2H), 7.45-7.34 (m, 4H), 7.09 (d, 1H), 5.48 (s, 2H), 3.85-3.38 (m, 8H).

LC/MS (Method 3, ESIpos): Rₜ=1.08 min; m/z=528 [M+H]⁺.

### Example 85

### 2-[3-(Morpholin-4-ylcarbonyl)benzyl]-6-{3-[4-(1,1,1-trifluoro-2-methylpropan-2-yl)phenyl]-1,2,4-oxadiazol-5-yl}pyridazin-3(2H)-one

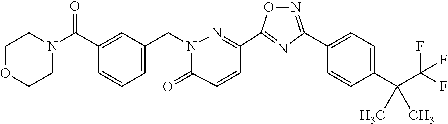 [SMILES: CC(C)(c1ccc(-c2noc(-c3ccc(=O)n(Cc4cccc(C(=O)N5CCOCC5)c4)n3)n2)cc1)C(F)(F)F]

Analogously to the process described in Example 71, 75 mg (0.150 mmol) of the compound from Example 39A and 20 μl (0.225 mmol) of morpholine gave 67 mg (78% of theory, purity 97%) of the title compound.

¹H NMR (400 MHz, CDCl₃, δ/ppm): 8.06 (d, 2H), 7.99 (d, 1H), 7.58 (d, 2H), 7.50 (m, 2H), 7.35 (m, 2H), 7.02 (d, 1H), 5.41 (s, 2H), 3.56 (m, 8H), 1.65 (s, 6H).

LC/MS (Method 7, ESIpos): Rₜ=1.18 min; m/z=554 [M+H]⁺.

### Example 86

### 2-{3-[(4-Methylpiperazin-1-yl)carbonyl]benzyl}-6-{3-[4-(1,1,1-trifluoro-2-methylpropan-2-yl)phenyl]-1,2,4-oxadiazol-5-yl}pyridazin-3(2H)-one

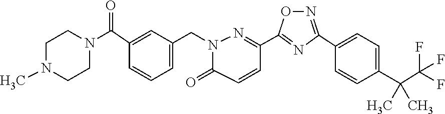 [SMILES: CN1CCN(C(=O)c2cccc(Cn3nc(-c4nc(-c5ccc(C(C)(C)C(F)(F)F)cc5)no4)ccc3=O)c2)CC1]

Analogously to the process described in Example 71, 75 mg (0.150 mmol) of the compound from Example 39A and 28 μl (0.225 mmol) of N-methylpiperazine gave 83 mg (97% of theory, purity 99%) of the title compound. Here, the mobile phase used for preparative thick-layer chromatography was dichloromethane/methanol 95:5.

¹H NMR (400 MHz, CDCl₃, δ/ppm): 8.13 (d, 2H), 8.06 (d, 1H), 7.65 (d, 2H), 7.60-7.54 (m, 2H), 7.44-7.35 (m, 2H), 7.08 (d, 1H), 5.48 (s, 2H), 3.79 (br. s, 2H), 3.43 (br. s, 2H), 2.48 (br. s, 2H), 2.34 (br. s, 2H), 2.31 (s, 3H), 1.63 (s, 6H).

LC/MS (Method 7, ESIpos): Rₜ=0.83 min; m/z=567 [M+H]⁺.

### Example 87

### 2-{3-[(4-Cyclopropylpiperazin-1-yl)carbonyl]benzyl}-6-{3-[4-(trifluoromethoxy)phenyl]-1,2,4-oxadiazol-5-yl}pyridazin-3(2H)-one

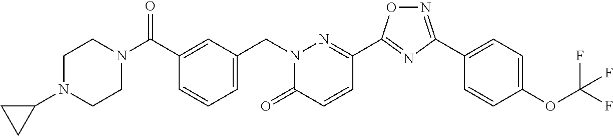 [SMILES: O=C(c1cccc(Cn2nc(-c3nc(-c4ccc(OC(F)(F)F)cc4)no3)ccc2=O)c1)N1CCN(C2CC2)CC1]

Analogously to the process described in Example 71, 80 mg (0.168 mmol) of the compound from Example 37A and 32 μl (0.252 mmol) of N-cyclopropylpiperazine gave 84 mg (83% of theory, purity 93%) of the title compound.

¹H NMR (400 MHz, CDCl₃, δ/ppm): 8.23-8.18 (m, 2H), 8.04 (d, 1H), 7.60-7.54 (m, 2H), 7.44-7.34 (m, 4H), 7.09 (d, 1H), 5.48 (s, 2H), 3.74 (br. s, 2H), 3.37 (br. s, 2H), 2.69 (br. s, 2H), 2.54 (br. s, 2H), 1.64-1.59 (m, 1H, obscured), 0.44 (m, 4H).

LC/MS (Method 3, ESIpos): Rₜ=0.88 min; m/z=567 [M+H]⁺.

### Example 88

### 2-{3-[(4-Cyclopropylpiperazin-1-yl)carbonyl]benzyl}-6-{3-[4-(1,1,1-trifluoro-2-methylpropan-2-yl)phenyl]-1,2,4-oxadiazol-5-yl}pyridazin-3(2H)-one

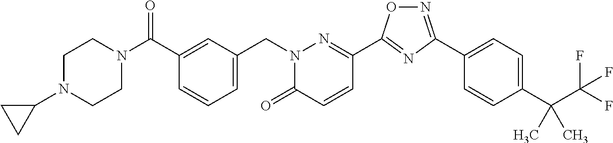 [SMILES: CC(C)(c1ccc(-c2noc(-c3ccc(=O)n(Cc4cccc(C(=O)N5CCN(C6CC6)CC5)c4)n3)n2)cc1)C(F)(F)F]

Analogously to the process described in Example 71, 75 mg (0.150 mmol) of the compound from Example 39A and 28 mg (0.225 mmol) of N-cyclopropylpiperazine gave 87 mg (92% of theory, purity 94%) of the title compound. Here, the mobile phase used for preparative thick-layer chromatography was dichloromethane/methanol 95:5.

¹H NMR (400 MHz, CDCl₃, δ/ppm): 8.12 (d, 2H), 8.05 (d, 1H), 7.64 (d, 2H), 7.59-7.53 (m, 2H), 7.43-7.35 (m, 2H), 7.08 (d, 1H), 5.47 (s, 2H), 3.73 (br. s, 2H), 3.35 (br. s, 2H), 2.68 (br. s, 2H), 2.53 (br. s, 2H), 1.62 (s, 6H), 1.63-1.61 (m, 1H, obscured), 0.47-0.37 (m, 4H).

LC/MS (Method 7, ESIpos): Rₜ=0.93 min; m/z=593 [M+H]⁺.

### Example 89

### 1-(3-{[6-oxo-3-{3-[4-(trifluoromethoxy)phenyl]-1,2,4-oxadiazol-5-yl}pyridazin-1(6H)-yl]methyl}phenyl)cyclopropyl acetate

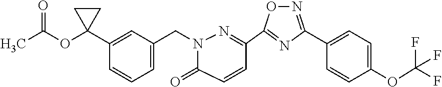 [SMILES: CC(=O)OC1(c2cccc(Cn3nc(-c4nc(-c5ccc(OC(F)(F)F)cc5)no4)ccc3=O)c2)CC1]

Analogously to the process described in Example 41, 243 mg (0.750 mmol) of the compound from Example 14A and 235 mg (0.825 mmol) of the compound from Example 1A gave 224 mg (56% of theory, purity 97%) of the title compound. Here, the chromatographic purification was carried out using the mobile phase cyclohexane/ethyl acetate 7:3.

¹H NMR (400 MHz, CDCl₃, δ/ppm): 8.22-8.18 (m, 2H), 8.02 (d, 1H), 7.46 (s, 1H), 7.40 (d, 1H), 7.36 (d, 2H), 7.30 (t, 1H), 7.24-7.20 (m, 1H), 7.07 (d, 1H), 5.44 (s, 2H), 2.06 (s, 3H), 1.33-1.27 (m, 2H), 1.26-1.21 (m, 2H).

LC/MS (Method 1, ESIpos): Rₜ=1.53 min; m/z=513 [M+H]⁺.

### Example 90

### 2-[3-(2-Hydroxy-2-methylpropyl)benzyl]-6-{3-[4-(trifluoromethoxy)phenyl]-1,2,4-oxadiazol-5-yl}pyridazin-3(2H)-one

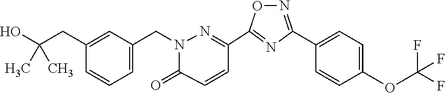 [SMILES: CC(C)(O)Cc1cccc(Cn2nc(-c3nc(-c4ccc(OC(F)(F)F)cc4)no3)ccc2=O)c1]

Analogously to the process described in Example 41, 130 mg (0.400 mmol) of the compound from Example 14A and 114 mg (0.440 mmol) of the compound from Example 2A gave 85 mg (42% of theory, purity 97%) of the title compound. Here, the chromatography on silica gel, as described above, was followed by a second purification step by preparative HPLC (Method 15).

¹H NMR (400 MHz, CDCl₃, δ/ppm): 8.20 (d, 2H), 8.03 (d, 1H), 7.43-7.34 (m, 4H), 7.30 (t, 1H), 7.18 (d, 1H), 7.08 (d, 1H), 5.46 (s, 2H), 2.78 (s, 2H), 1.22 (s, 6H).

LC/MS (Method 1, ESIpos): Rₜ=1.44 min; m/z=487 [M+H]⁺.

### Example 91

### 2-[3-(2-Hydroxy-2-methylpropyl)benzyl]-6-{3-[4-(1,1,1-trifluoro-2-methylpropan-2-yl)phenyl]-1,2,4-oxadiazol-5-yl}pyridazin-3(2H)-one

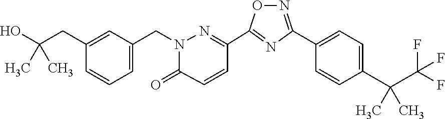 [SMILES: CC(C)(O)Cc1cccc(Cn2nc(-c3nc(-c4ccc(C(C)(C)C(F)(F)F)cc4)no3)ccc2=O)c1]

Analogously to the process described in Example 41, 140 mg (0.400 mmol) of the compound from Example 15A and 114 mg (0.440 mmol) of the compound from Example 2A gave 108 mg (52% of theory) of the title compound. Here, the chromatographic purification on silica gel was carried out using the mobile phase cyclohexane/ethyl acetate 2:3. This was followed by a second purification step by preparative HPLC (Method 15).

¹H NMR (400 MHz, CDCl₃, δ/ppm): 8.14 (d, 2H), 8.04 (d, 1H), 7.65 (d, 2H), 7.42-7.38 (m, 2H), 7.30 (t, 1H), 7.18 (d, 1H), 7.08 (d, 1H), 5.46 (s, 2H), 2.77 (s, 2H), 1.63 (s, 6H), 1.22 (s, 6H).

LC/MS (Method 3, ESIpos): Rₜ=1.29 min; m/z=513 [M+H]⁺.

### Example 92

### 2-{3-[1-(Hydroxymethyl)cyclopropyl]benzyl}-6-{3-[4-(trifluoromethoxy)phenyl]-1,2,4-oxadiazol-5-yl}pyridazin-3(2H)-one

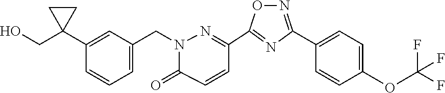 [SMILES: O=c1ccc(-c2nc(-c3ccc(OC(F)(F)F)cc3)no2)nn1Cc1cccc(C2(CO)CC2)c1]

Analogously to the process described in Example 44, 156 mg (0.243 mmol) of the compound from Example 40A and 291 μl (0.291 mmol) of a 1 M solution of tetra-n-butylammonium fluoride (TBAF) in THF gave 73 mg (62% of theory) of the title compound. Here, the chromatographic purification was carried out using the mobile phase cyclohexane/ethyl acetate 1:1.

¹H NMR (400 MHz, CDCl₃, δ/ppm): 8.23-8.18 (m, 2H), 8.02 (d, 1H), 7.56 (s, 1H), 7.40-7.27 (m, 5H), 7.08 (d, 1H), 5.45 (s, 2H), 3.68 (d, 2H), 1.52-1.46 (m, 1H), 0.92-1.85 (m, 4H).

LC/MS (Method 7, ESIpos): Rₜ=1.24 min; m/z=485 [M+H]⁺.

### Example 93

### 2-{3-[1-(Hydroxymethyl)cyclopropyl]benzyl}-6-{3-[4-(1,1,1-trifluoro-2-methylpropan-2-yl)phenyl]-1,2,4-oxadiazol-5-yl}pyridazin-3(2H)-one

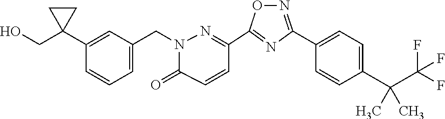 [SMILES: CC(C)(c1ccc(-c2noc(-c3ccc(=O)n(Cc4cccc(C5(CO)CC5)c4)n3)n2)cc1)C(F)(F)F]

Analogously to the process described in Example 44, 174 mg (0.261 mmol) of the compound from Example 41A and 313 μl (0.313 mmol) of a 1 M solution of tetra-n-butylammonium fluoride (TBAF) in THF gave 76 mg (57% of theory) of the title compound. Here, the chromatographic purification was carried out using the mobile phase cyclohexane/ethyl acetate 1:1, and the product obtained in this manner was then triturated with a little pentane, then filtered off with suction and dried under reduced pressure.

¹H NMR (400 MHz, CDCl₃, δ/ppm): 8.14 (d, 2H), 8.04 (d, 1H), 7.65 (d, 2H), 7.56 (s, 1H), 7.40-7.36 (m, 1H), 7.35-7.27 (m, 2H), 7.08 (d, 1H), 5.45 (s, 2H), 3.68 (d, 2H), 1.63 (s, 6H), 1.53-1.48 (m, 1H), 0.92-0.85 (m, 4H).

LC/MS (Method 7, ESIpos): Rₜ=1.29 min; m/z=511 [M+H]⁺.

### Example 94

### 1-(4-{[6-oxo-3-{3-[4-(trifluoromethoxy)phenyl]-1,2,4-oxadiazol-5-yl}pyridazin-1(6H)-yl]methyl}pyridin-2-yl)piperidine-4-carbonitrile

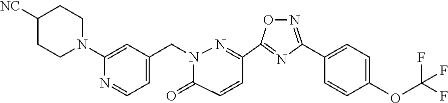 [SMILES: N#CC1CCN(c2cc(Cn3nc(-c4nc(-c5ccc(OC(F)(F)F)cc5)no4)ccc3=O)ccn2)CC1]

Analogously to the process described in Example 45, 140 mg (0.312 mmol) of the compound from Example 42A and 687 mg (6.23 mmol) of 4-cyanopiperidine gave 62 mg (38% of theory) of the title compound. In this case, the reaction time in the microwave oven was 45 min (instead of 3 h).

¹H NMR (400 MHz, CDCl₃, δ/ppm): 8.20 (d, 2H), 8.15 (d, 1H), 8.07 (d, 1H), 7.37 (d, 2H), 7.12 (d, 1H), 6.78 (s, 1H), 6.72 (d, 1H), 5.36 (s, 2H), 3.85 (ddd, 2H), 3.49 (ddd, 2H), 2.87 (tt, 1H), 2.04-1.96 (m, 2H), 1.96-1.86 (m, 2H).

LC/MS (Method 3, ESIpos): Rₜ=1.06 min; m/z=524 [M+H]⁺.

### Example 95

### 1-(4-{[6-oxo-3-{3-[4-(1,1,1-trifluoro-2-methylpropan-2-yl)phenyl]-1,2,4-oxadiazol-5-yl}pyridazin-1(6H)-yl]methyl}pyridin-2-yl)piperidine-4-carbonitrile

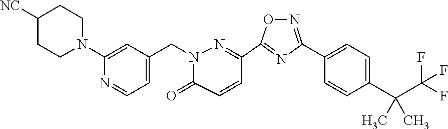 [SMILES: CC(C)(c1ccc(-c2noc(-c3ccc(=O)n(Cc4ccnc(N5CCC(C#N)CC5)c4)n3)n2)cc1)C(F)(F)F]

Analogously to the process described in Example 45, 119 mg (0.250 mmol) of the compound from Example 43A and 551 mg (5.00 mmol) of 4-cyanopiperidine gave 42 mg (31% of theory) of the title compound. In this case, the reaction time in the microwave oven was 1.5 h (instead of 3 h).

¹H NMR (400 MHz, CDCl₃, δ/ppm): 8.16-8.12 (m, 3H), 8.09 (d, 1H), 7.66 (d, 2H), 7.11 (d, 1H), 6.78 (s, 1H), 6.72 (d, 1H), 5.36 (s, 2H), 3.86 (ddd, 2H), 3.48 (ddd, 2H), 2.86 (tt, 1H), 2.05-1.96 (m, 2H), 1.96-1.86 (m, 2H), 1.63 (s, 6H).

LC/MS (Method 3, ESIpos): Rₜ=1.08 min; m/z=550 [M+H]⁺.

### Example 96

### 2-{[2-(4-Cyclopropylpiperazin-1-yl)pyridin-4-yl]methyl}-6-{3-[4-(trifluoromethoxy)phenyl]-1,2,4-oxadiazol-5-yl}pyridazin-3(2H)-one

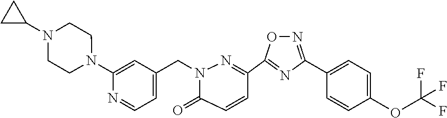 [SMILES: O=c1ccc(-c2nc(-c3ccc(OC(F)(F)F)cc3)no2)nn1Cc1ccnc(N2CCN(C3CC3)CC2)c1]

Analogously to the process described in Example 41, 162 mg (0.500 mmol) of the compound from Example 14A and 151 mg (0.600 mmol) of the compound from Example 5A gave 154 mg (57% of theory) of the title compound. In this case, the reaction time in the alkylation partial step was 1 h (instead of 2 h).

¹H NMR (400 MHz, CDCl₃, δ/ppm): 8.20 (d, 2H), 8.14 (d, 1H), 8.06 (d, 1H), 7.37 (d, 2H), 7.11 (d, 1H), 6.75 (s, 1H), 6.67 (d, 1H), 5.36 (s, 2H), 3.54-3.50 (m, 4H), 2.73-2.69 (m, 4H), 1.64-1.60 (m, 1H, obscured), 0.50-0.45 (m, 4H).

LC/MS (Method 7, ESIpos): Rₜ=0.84 min; m/z=540 [M+H]⁺.

### Example 97

### 2-{[2-(4-Cyclopropylpiperazin-1-yl)pyridin-4-yl]methyl}-6-{3-[4-(1,1,1-trifluoro-2-methylpropan-2-yl)phenyl]-1,2,4-oxadiazol-5-yl}pyridazin-3(2H)-one

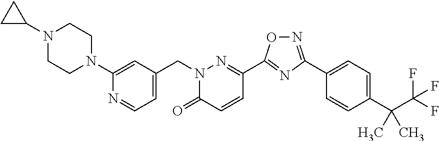 [SMILES: CC(C)(c1ccc(-c2noc(-c3ccc(=O)n(Cc4ccnc(N5CCN(C6CC6)CC5)c4)n3)n2)cc1)C(F)(F)F]

Analogously to the process described in Example 41, 88 mg (0.250 mmol) of the compound from Example 15A and 76 mg (0.300 mmol) of the compound from Example 5A gave 91 mg (64% of theory) of the title compound. In this case, the reaction time in the alkylation partial step was three days (instead of 2 h).

¹H NMR (400 MHz, CDCl₃, δ/ppm): 8.16-8.12 (m, 3H), 8.08 (d, 1H), 7.65 (d, 2H), 7.11 (d, 1H), 6.75 (s, 1H), 6.68 (d, 1H), 5.36 (s, 2H), 3.55-3.50 (m, 4H), 2.73-2.68 (m, 4H), 1.64-1.61 (m, 1H, obscured), 1.63 (s, 6H), 0.50-0.44 (m, 4H).

LC/MS (Method 3, ESIpos): Rₜ=0.91 min; m/z=566 [M+H]⁺.

### Example 98

### 2-[(6-Chloropyridin-3-yl)methyl]-6-{3-[4-(trifluoromethoxy)phenyl]-1,2,4-oxadiazol-5-yl}pyridazin-3(2H)-one

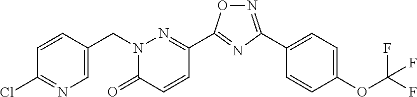 [SMILES: O=c1ccc(-c2nc(-c3ccc(OC(F)(F)F)cc3)no2)nn1Cc1ccc(Cl)nc1]

Analogously to the process described in Example 41, 120 mg (0.370 mmol) of the compound from Example 14A and 78 mg (0.481 mmol) of 2-chloro-5-(chloromethyl)pyridine gave 83 mg (50% of theory) of the title compound. In this case, the reaction time in the alkylation partial step was 4 h (instead of 2 h). The mobile phase used for the chromatographic purification was cyclohexane/ethyl acetate 7:3.

¹H NMR (400 MHz, CDCl₃, δ/ppm): 8.60 (d, 1H), 8.20 (d, 2H), 8.06 (d, 1H), 7.88 (dd, 1H), 7.35 (m, 3H), 7.11 (d, 1H), 5.45 (s, 2H).

LC/MS (Method 3, ESIpos): Rₜ=1.23 min; m/z=450 [M+H]⁺.

### Example 99

### 2-[(6-Chloropyridin-3-yl)methyl]-6-{3-[4-(1,1,1-trifluoro-2-methylpropan-2-yl)phenyl]-1,2,4-oxadiazol-5-yl}pyridazin-3(2H)-one

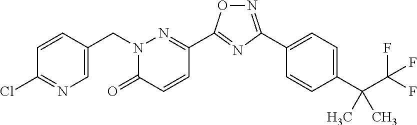 [SMILES: CC(C)(c1ccc(-c2noc(-c3ccc(=O)n(Cc4ccc(Cl)nc4)n3)n2)cc1)C(F)(F)F]

With ice cooling, 40 mg (0.975 mmol, 60% in mineral oil) of sodium hydride were added to a mixture of 263 mg (0.750 mmol) of the compound from Example 15A in 5 ml of DMF. After 15 min of stirring, 158 mg (0.975 mmol) of 2-chloro-5-(chloromethyl)pyridine were added, and the mixture was stirred at RT for 3 days. A further 20 mg (0.478 mmol) of sodium hydride were then added, and the mixture was stirred at 70° C. for 1.5 h. 50 ml of water were then added slowly, and the mixture was extracted twice with in each case 30 ml of ethyl acetate. The combined organic phases were washed once with 50 ml of saturated sodium chloride solution, dried over sodium sulphate, filtered and concentrated. The residue was purified by column chromatography (Biotage, silica gel, mobile phase cyclohexane/ethyl acetate 1:1). Drying under high vacuum gave 164 mg (46% of theory) of the title compound.

¹H NMR (400 MHz, CDCl₃, δ/ppm): 8.60 (d, 1H), 8.14 (d, 2H), 8.07 (d, 1H), 7.88 (dd, 1H), 7.66 (d, 2H), 7.33 (d, 1H), 7.10 (d, 1H), 5.45 (s, 2H), 1.63 (s, 6H).

LC/MS (Method 3, ESIpos): Rₜ=1.25 min; m/z=476 [M+H]⁺.

### Example 100

### 2-{[6-(Methylamino)pyridin-3-yl]methyl}-6-{3-[4-(trifluoromethoxy)phenyl]-1,2,4-oxadiazol-5-yl}pyridazin-3(2H)-one

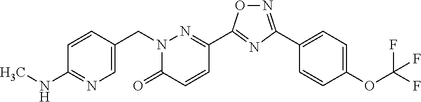 [SMILES: CNc1ccc(Cn2nc(-c3nc(-c4ccc(OC(F)(F)F)cc4)no3)ccc2=O)cn1]

Analogously to the process described in Example 59, 65 mg (0.144 mmol) of the compound from Example 98 and 1.8 ml (14.4 mmol) of a 33% strength solution of methylamine in ethanol were converted into 10 mg (16% of theory) of the title compound. Here, purification by preparative HPLC was carried out according to Method 13.

¹H NMR (400 MHz, CDCl₃, δ/ppm): 8.27 (d, 1H), 8.20 (d, 2H), 8.01 (d, 1H), 7.72 (dd, 1H), 7.37 (d, 2H), 7.06 (d, 1H), 6.39 (d, 1H), 5.33 (s, 2H), 5.13 (br. s, 1H), 2.91 (d, 3H).

LC/MS (Method 3, ESIpos): Rₜ=0.81 min; m/z=445 [M+H]⁺.

### Example 101

### 2-{[6-(Methylamino)pyridin-3-yl]methyl}-6-{3-[4-(1,1,1-trifluoro-2-methylpropan-2-yl)phenyl]-1,2,4-oxadiazol-5-yl}pyridazin-3(2H)-one

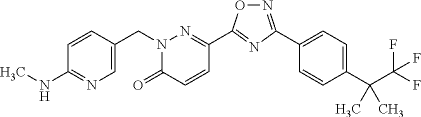 [SMILES: CNc1ccc(Cn2nc(-c3nc(-c4ccc(C(C)(C)C(F)(F)F)cc4)no3)ccc2=O)cn1]

Analogously to the process described in Example 59, 138 mg (0.290 mmol) of the compound from Example 99 and 3.6 ml (29.1 mmol) of a 33% strength solution of methylamine in ethanol were converted into 18 mg (13% of theory) of the title compound. Here, purification was carried out by two preparative HPLCs (first according to Method 13, then according to Method 19).

¹H NMR (400 MHz, CDCl₃, δ/ppm): 8.29 (d, 1H), 8.14 (d, 2H), 8.03 (d, 1H), 7.71 (dd, 1H), 7.65 (d, 2H), 7.06 (d, 1H), 6.38 (d, 1H), 5.33 (s, 2H), 4.89 (br. s, 1H), 2.91 (d, 3H), 1.63 (s, 6H).

LC/MS (Method 1, ESIpos): Rₜ=1.14 min; m/z=471 [M+H]⁺.

### B. ASSESSMENT OF PHARMACOLOGICAL EFFICACY

The pharmacological activity of the compounds according to the invention can be demonstrated by in vitro and in vivo studies, as known to the person skilled in the art. The usefulness of the substances according to the invention can be illustrated by way of example by in vitro (tumour) cell experiments and in vivo tumour models such as are described below. The connection between an inhibition of the HIF transcription activity and the inhibition of tumour growth is demonstrated by numerous studies described in the literature (cf. e.g. Warburg, 1956; Semenza, 2007).

### B-1. HIF-Luciferase Assay

HCT 116 cells were transfected in a stable manner with a plasmid which contained a luciferase reporter under the control of an HIF-responsive sequence. These cells were sown in microtitre plates [20000 cells/cavity in RPMI 1640 medium with 10% foetal calf serum (FCS) and 100 μg/ml of hygromycin]. Incubation was carried out overnight under standard conditions (5% CO₂, 21% O₂, 37° C., moistened). The following morning the cells were incubated with various concentrations of the test substances (0-10 μmol/l) in a hypoxia chamber (1% O₂). After 24 h, Bright Glo reagent (Promega, Wisconsin, USA) was added in accordance with the manufacturer's instructions, and after 5 min the luminescence was measured. Cells which were incubated under normoxia served as background controls.

The IC₅₀ values from this assay for representative working examples are given in the table below (in some cases as means of up to four individual determinations):

| Example No. | IC₅₀ [nmol/l] |
|---|---|
| 1 | 45 |
| 2 | 40 |
| 4 | 50 |
| 7 | 20 |
| 9 | 1.5 |
| 10 | 1 |
| 11 | 2 |
| 12 | 1.5 |
| 13 | 2 |
| 14 | 2 |
| 15 | 2 |
| 16 | 5 |
| 19 | 30 |
| 21 | 30 |
| 22 | 20 |
| 23 | 15 |
| 25 | 5 |
| 27 | 1 |
| 28 | 2 |
| 30 | 2 |
| 32 | 2 |
| 33 | 6 |
| 34 | 4 |
| 35 | 20 |
| 36 | 5 |
| 37 | 10 |
| 38 | 3 |
| 39 | 5 |
| 40 | 2.5 |
| 41 | 5 |
| 42 | 8 |
| 43 | 10 |
| 44 | 15 |
| 45 | 5 |
| 47 | 40 |
| 48 | 50 |
| 49 | 3 |
| 51 | 2 |
| 52 | 5 |
| 53 | 4 |
| 54 | 4 |
| 55 | 20 |
| 59 | 12.5 |
| 60 | 20 |
| 63 | 30 |
| 65 | 200 |
| 66 | 1.5 |
| 70 | 60 |
| 73 | 50 |
| 74 | 10 |
| 76 | 20 |
| 78 | 5 |
| 80 | 5 |
| 82 | 15 |
| 84 | 20 |
| 86 | 40 |
| 87 | 5 |
| 89 | 5 |
| 90 | 6 |
| 91 | 40 |
| 92 | 10 |
| 93 | 25 |
| 94 | 20 |
| 95 | 40 |
| 96 | 1.5 |
| 97 | 3 |
| 100 | 6 |
| 101 | 15 |

### B-2. Suppression of HIF Target Genes In Vitro:

Human bronchial carcinoma cells (H460 and A549 cell lines) were incubated for 16 h with variable concentrations of the test substances (1 nM to 10 μM) under normoxic conditions and under a 1% oxygen partial pressure (see HIF-luciferase assay). The total RNA was isolated from the cells and transcribed into cDNA and the mRNA expression of HIF target genes was analysed in real-time PCR. Active test substances already lower the mRNA expression of the HIF target genes compared with untreated cells under normoxic conditions, but above all under hypoxic conditions.

### B-3. Human Xenograft Tumour Model:

Human tumour xenograft models in immunodeficient mice were used to assess the substances. For this purpose, tumour cells were cultured in vitro and implanted subcutaneously, or tumour xenotransplant pieces were transplanted further subcutaneously. The animals were treated by oral, subcutaneous or intraperitoneal therapy after the tumour had been established. The activity of the test substances was analysed in monotherapy and in combination therapy with other pharmacological active substances. In addition, the tumour inhibitory potency of test substances on tumours of advanced size (approx. 100 mm²) was characterized. The state of health of the animals was checked daily, and the treatments were performed in accordance with animal welfare regulations. The tumour area was measured with slide gauges (length L, breadth B=shorter dimension). The tumour volume was calculated by the formula (L×B²)/2. The inhibition in tumour growth was determined at the end of the study as the T/C ratio of the tumour areas and tumour weights and as the TGI value (tumour growth inhibition, calculated from the formula [1−(T/C)]×100) (T=tumour size in the treated group; C=tumour size in the untreated control group).

The influence of the test substances on the tumour vessel architecture and the blood flow within the tumour was identified with the aid of computer microtomography and ultrasound microstudies on treated and untreated tumour-carrying mice.

### B-4. Determination of Pharmacokinetic Parameters Following Intravenous and Peroral Administration:

The substance to be examined was administered to animals (for example mice or rats) intravenously as a solution (for example in corresponding plasma with a small addition of DMSO or in a PEG/ethanol/water mixture), and peroral administration was effected as a solution (for example in Solutol/ethanol/water or PEG/ethanol/water mixtures) or as a suspension (e.g. in tylose), in each case via a gavage. After administration of the substance, blood was taken from the animals at fixed times. The blood was heparinized, then plasma was obtained therefrom by centrifugation. The substance was quantified analytically in the plasma via LC-MS/MS. From the plasma concentration/time plots determined in this way, using an internal standard and with the aid of a validated computer program, the pharmacokinetic parameters, such as AUC (area under the concentration/time curve), Cₘₐₓ (maximum plasma concentration), t_{1/2} (half life), V_{SS} (distribution volume) and CL (clearance), and the absolute and relative bioavailability F and Fᵣₑₗ (i.v./p.o. comparison or comparison of suspension to solution after p.o. administration), were calculated.

### C. WORKING EXAMPLES OF PHARMACEUTICAL COMPOSITIONS

The compounds according to the invention can be converted to pharmaceutical formulations as follows:

### Tablet:

### Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm.

### Production:

The mixture of compound according to the invention, lactose and starch is granulated with a 5% solution (w/w) of the PVP in water. The granules are dried and mixed with the magnesium stearate for 5 minutes. This mixture is pressed with a conventional tableting press (for tablet dimensions see above). The guide value used for the pressing is a pressing force of 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

A single dose of 100 mg of the compound according to the invention corresponds to 10 ml of oral suspension.

### Production:

The Rhodigel is suspended in ethanol and the compound according to the invention is added to the suspension. The water is added while stirring. The mixture is stirred for about 6 h until swelling of the Rhodigel is complete.

Solution which can be Administered Orally:

### Composition:

500 mg of the compound according to the invention, 2.5 g of polysorbate and 97 g of polyethylene glycol 400. A single dose of 100 mg of the compound according to the invention corresponds to 20 g of oral solution.

### Production:

The compound according to the invention is suspended in the mixture of polyethylene glycol and polysorbate while stirring. The stirring operation is continued until dissolution of the compound according to the invention is complete.

i.v. Solution:

The compound according to the invention is dissolved in a concentration below the saturation solubility in a physiologically acceptable solvent (e.g. isotonic saline, glucose solution 5% and/or PEG 400 solution 30%). The solution is subjected to sterile filtration and dispensed into sterile and pyrogen-free injection vessels.

### D. LITERATURE REFERENCES

Globocan 2002 Report IARC International Agency for Research on Cancer: Globocan 2002, http://www-dep.iarc.fr/globocan/downloads.htm
American Cancer Society, Cancer Facts and Figures 2005 American Cancer Society: Cancer Facts and Figures. 2007, http://www.cancer.org/docroot/STT/content/STT_{—}1x_CancerFacts_Figures_{—}2007.asp
Gibbs J B, 2000 Gibbs J B: Mechanism-based target identification and drug discovery in cancer research, Science 2000, 287 (5460), 1969-1973.
Semenza and Wang, 1992 Semenza G L, Wang G L: A nuclear factor induced by hypoxia via de novo protein synthesis binds to the human erythropoietin gene enhancer at a site required for transcriptional activation, Mol. Cell. Biol. 1992, 12 (12), 5447-5454.
Wang and Semenza, 1995 Wang G L, Semenza G L: Purification and characterization of hypoxia-inducible factor 1, J. Biol. Chem. 1995, 270 (3), 1230-1237.
Wang, Jiang et al., 1995 Wang G L, Jiang B H, Rue E A, Semenza G L: Hypoxia-inducible factor 1 is a basic-helix-loop-helix-PAS heterodimer regulated by cellular O₂ tension, PNAS 1995, 92 (12), 5510-5514.
Jiang, Rue et al., 1996 Jiang B H, Rue E, Wang G L, Roe R, Semenza G L: Dimerization, DNA binding, and transactivation properties of hypoxia-inducible factor 1, J. Biol. Chem. 1996, 271 (30), 17771-17778.
Makino, Cao et al., 2001 Makino Y, Cao R, Svensson K, Bertilsson G, Asman M, Tanaka H, Cao Y, Poellinger L: Nature 2001, 414 (6863), 550-554.
Jiang, Semenza et al., 1996 Jiang B H, Semenza G L, Bauer C, Marti H H: Hypoxia-inducible factor 1 levels vary exponentially over a physiologically relevant range of O₂ tension, Am. J. Physiol. 1996, 271, 1172-1180.
Maxwell, Wiesener et al., 1999 Maxwell P H, Wiesener M S, Chang G W, Clifford S C, Vaux E C, Cockman M E, Wykoff C C, Ratcliffe P J: The tumour suppressor protein VHL targets hypoxia-inducible factors for oxygen-dependent proteolysis, Nature 1999, 399 (6733), 271-275.
Hirota and Semenza, 2006 Hirota K, Semenza G L: Regulation of angiogenesis by hypoxia-inducible factor 1, Crit. Rev. Oncol. Hematol. 2006, 59 (1), 15-26.
Chen, Zhao et al., 2003 Chen J, Zhao S, Nakada K, Kuge Y, Tamaki N, Okada F, Wang J, Shindo M, Higashino F, Takeda K, Asaka M, Katoh H, Sugiyama T, Hosokawa M, Kobayashi M: Dominant-negative hypoxia-inducible factor-1 alpha reduces tumorigenicity of pancreatic cancer cells through the suppression of glucose metabolism, Am. J. Pathol. 2003, 162 (4), 1283-1291.
Stoeltzing, McCarty et al., 2004 Stoeltzing O, McCarty M F, Wey J S, Fan F, Liu W, Belcheva A, Bucana C D, Semenza G L, Ellis L M: Role of hypoxia-inducible factor-1 alpha in gastric cancer cell growth, angiogenesis, and vessel maturation, J. Natl. Cancer Inst. 2004, 96 (12), 946-956.
Li, Lin et al., 2005 Li L, Lin X, Staver M, Shoemaker A, Semizarov D, Fesik S W, Shen Y: Evaluating hypoxia-inducible factor-1 alpha as a cancer therapeutic target via inducible RNA interference in vivo, Cancer Res. 2005, 65 (16), 7249-7258.
Mizukami, Jo et al., 2005 Mizukami Y, Jo W S, Duerr E M, Gala M, Li J, Zhang X, Zimmer M A, Iliopoulos O, Zukerberg L R, Kohgo Y, Lynch M P, Rueda B R, Chung D C: Induction of interleukin-8 preserves the angiogenic response in HIF-1alpha-deficient colon cancer cells, Nat. Med. 2005, 11 (9), 992-997.
Li, Shi et al., 2006 Li J, Shi M, Cao Y, Yuan W, Pang T, Li B, Sun Z, Chen L, Zhao R C: Knockdown of hypoxia-inducible factor-1 alpha in breast carcinoma MCF-7 cells results in reduced tumor growth and increased sensitivity to methotrexate, Biochem. Biophys. Res. Commun. 2006, 342, 1341-1351.
Semenza, 2007 Semenza G L: Drug Discov. Today 2007, 12 (19-20), 853-859.
Weidemann and Johnson, 2008 Weidemann A, Johnson R S: Cell Death and Differentiation 2008, 15, 621-627.
Aiello et al., 1994 Aiello et al.: New Engl. J. Med. 1994, 331, 1480.
Peer et al., 1995 Peer et al.: Lab. Invest. 1995, 72, 638.
Lopez et al., 1996 Lopez et al.: Invest. Ophthalmol. Vis. Sci. 1996, 37, 855.
Warburg, 1956 Warburg O: Science 1956, 123 (3191), 309-314.
Girgis et al., 2012 Girgis C M et al.: Trends Endocrinol. Metab. 2012, in press (electronic pre-publication July 2012).

1. Compound of the formula (I) 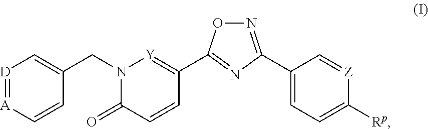
in which
either (i)
A represents C—R^{A}
and
D represents CH or N
or (ii)
A represents CH or N
and
D represents C—R^{D},
where R^{A} represents chlorine, cyano, nitro, amino, (C₁-C₄)-alkyl, (C₁-C₄)-alkoxy or mono-(C₁-C₄)-alkylamino,
where (C₁-C₄)-alkyl, (C₁-C₄)-alkoxy and mono-(C₁-C₄)-alkylamino may be substituted by hydroxy or up to three times by fluorine,
and
R^{D} represents (C₁-C₆)-alkyl, (C₃-C₆)-cycloalkyl, (C₁-C₄)-alkoxycarbonyl or a group of the formula —NR¹R² or —C(═O)—NR³R⁴,
where (C₁-C₆)-alkyl is substituted by hydroxy or (C₁-C₄)-alkylcarbonyloxy and may additionally be substituted up to three times by fluorine
and
(C₃-C₆)-cycloalkyl is substituted by hydroxy, hydroxy-(C₁-C₄)-alkyl or (C₁-C₄)-alkylcarbonyloxy,
and where
R¹ and R² are attached to one another and together with the nitrogen atom to which they are attached form a 4- to 6-membered heterocycle which may contain a further heteroatom from the group consisting of N(R⁵), O, S and S(O)₂ and which may be substituted up to two times by identical or different radicals selected from the group consisting of fluorine, cyano, (C₁-C₄)-alkyl hydroxy, methoxy and ethoxy,
where (C₁-C₄)-alkyl for its part may be substituted by hydroxy or up to three times by fluorine
and where
R⁵ represents (C₁-C₄)-alkyl which may be substituted up to three times by fluorine or represents (C₃-C₆)-cycloalkyl, (C₁-C₄)-alkylcarbonyl or (C₁-C₄)-alkoxycarbonyl,
and
R³ and R⁴ independently of one another represent hydrogen or (C₁-C₄)-alkyl which may be substituted by hydroxy, methoxy, ethoxy or phenyl or up to three times by fluorine
or
R³ and R⁴ are attached to one another and have the meanings of R¹ and R²,
Y represents CH or N,
Z represents C—R^{m} or N, where
R^{m} represents hydrogen, fluorine, chlorine, methyl or trifluoromethyl,
and
R^{p} represents halogen, cyano, pentafluorothio, (C₁-C₆)-alkyl, (C₁-C₆)-alkoxy, (C₁-C₆)-alkylthio, (C₁-C₆)-alkylsulphonyl, (C₃-C₆)-cycloalkyl or 4- to 6-membered heterocyclyl,
where (C₁-C₆)-alkyl, (C₁-C₆)-alkoxy, (C₁-C₆)-alkylthio and (C₁-C₆)-alkylsulphonyl for their part may be substituted by a radical selected from the group consisting of hydroxy, methoxy and ethoxy and up to six times by fluorine
and
(C₃-C₆)-cycloalkyl and 4- to 6-membered heterocyclyl for their part may be substituted up to two times by identical or different radicals selected from the group consisting of fluorine, (C₁-C₄)-alkyl, trifluoromethyl, hydroxy, methoxy and ethoxy,
and their salts, solvates and solvates of the salts,
except for the compounds
1-(4-chlorobenzyl)-5-[3-(4-methylphenyl)-1,2,4-oxadiazol-5-yl]pyridin-2(1H)-one,
5-[3-(4-chlorophenyl)-1,2,4-oxadiazol-5-yl]-1-(4-methylbenzyl)pyridin-2(1H)-one,
5-[3-(4-methoxyphenyl)-1,2,4-oxadiazol-5-yl]-1-(4-methylbenzyl)pyridin-2(1H)-one
and
2-(4-methoxybenzyl)-6-[3-(4-methylphenyl)-1,2,4-oxadiazol-5-yl]pyridazin-3(2H)-one.

2. Compound of the formula (I) according to claim 1 in which
either (i)
A represents C—R^{A}
and
D represents CH or N
or (ii)
A represents CH or N
and
D represents C—R^{D},
where R^{A} represents chlorine, cyano, amino, (C₁-C₄)-alkyl, (C₁-C₄)-alkoxy or mono-(C₁-C₄)-alkylamino,
where (C₁-C₄)-alkyl, (C₁-C₄)-alkoxy and mono-(C₁-C₄)-alkylamino may be substituted by hydroxy or up to three times by fluorine,
and
R^{D} represents (C₁-C₆)-alkyl, (C₃-C₆)-cycloalkyl or a group of the formula —NR¹R² or —C(═O)—NR³R⁴,
where (C₁-C₆)-alkyl is substituted by hydroxy or (C₁-C₄)-alkylcarbonyloxy and may additionally be substituted up to three times by fluorine
and
(C₃-C₆)-cycloalkyl is substituted by hydroxy, hydroxy-(C₁-C₄)-alkyl or (C₁-C₄)-alkylcarbonyloxy,
and where
R¹ and R² are attached to one another and together with the nitrogen atom to which they are attached form a 4- to 6-membered heterocycle which may contain a further heteroatom from the group consisting of N(R⁵) and O and which may be substituted up to two times by identical or different radicals selected from the group consisting of fluorine, cyano, hydroxy, methoxy and ethoxy,
where (C₁-C₄)-alkyl for its part may be substituted by hydroxy or up to three times by fluorine
and where
R⁵ represents (C₁-C₄)-alkyl which may be substituted up to three times by fluorine or represents (C₃-C₆)-cycloalkyl, (C₁-C₄)-alkylcarbonyl or (C₁-C₄)-alkoxycarbonyl,
and
R³ and R⁴ independently of one another represent hydrogen or (C₁-C₄)-alkyl which may be substituted by hydroxy, methoxy or ethoxy or up to three times by fluorine
or
R³ and R⁴ are attached to one another and have the meanings of R¹ and R²,
Y represents CH or N,
Z represents C—R^{m} or N, where
R^{m} represents hydrogen or fluorine,
and
R^{p} represents cyano, pentafluorothio, trifluoromethyl, (C₂-C₆)-alkyl, trifluoromethoxy, (C₂-C₆)-alkoxy, trifluoromethylthio, (C₂-C₆)-alkylthio, (C₃-C₆)-cycloalkyl or 4- to 6-membered heterocyclyl,
where (C₂-C₆)-alkyl, (C₂-C₆)-alkoxy and (C₂-C₆)-alkylthio for their part may be substituted by a radical selected from the group consisting of hydroxy, methoxy and ethoxy and up to six times by fluorine
and
(C₃-C₆)-cycloalkyl and 4- to 6-membered heterocyclyl for their part may be substituted up to two times by identical or different radicals selected from the group consisting of fluorine, (C₁-C₄)-alkyl, trifluoromethyl, hydroxy, methoxy and ethoxy,
and their salts, solvates and solvates of the salts.

3. Compound of the formula (I) according to claim 1 in which
either (i)
A represents C—R^{A}
and
D represents CH or N
or (ii)
A represents CH or N
and
D represents C—R^{D},
where R^{A} represents (C₁-C₄)-alkyl, amino or mono-(C₁-C₄)-alkylamino,
where (C₁-C₄)-alkyl and mono-(C₁-C₄)-alkylamino may be substituted by hydroxy or up to three times by fluorine,
and
R^{D} represents (C₁-C₄)-alkyl, cyclopropyl, cyclobutyl or a group of the formula —NR¹R² or —C(═O)—NR³R⁴,
where (C₁-C₄)-alkyl is substituted by hydroxy or acetoxy and may additionally be substituted up to three times by fluorine
and cyclopropyl and cyclobutyl are substituted by hydroxy, hydroxymethyl or acetoxy,
and where
R¹ and R² are attached to one another and together with the nitrogen atom to which they are attached form a 4- to 6-membered heterocycle which may contain a further heteroatom from the group consisting of N(R⁵) and O and which may be substituted by a radical selected from the group consisting of cyano, methyl, hydroxy and methoxy, where
R⁵ represents (C₁-C₄)-alkyl which may be substituted up to three times by fluorine or represents cyclopropyl, cyclobutyl or acetyl,
and
R³ and R⁴ independently of one another represent hydrogen or (C₁-C₄)-alkyl which may be substituted by hydroxy
or
R³ and R⁴ are attached to one another and have the meanings of R¹ and R²,
Y represents CH or N,
Z represents C—R^{m} or N, where
R^{m} represents hydrogen or fluorine,
and
R^{p} represents pentafluorothio, trimethylsilyl, (C₂-C₄)-alkyl, trifluoromethoxy, (C₂-C₄)-alkoxy, trifluoromethylthio, (C₂-C₄)-alkylthio, cyclopropyl or cyclobutyl,
where (C₂-C₄)-alkyl, (C₂-C₄)-alkoxy and (C₂-C₄)-alkylthio may be substituted by hydroxy or methoxy or up to three times by fluorine,
and
cyclopropyl and cyclobutyl may be substituted by a radical selected from the group consisting of fluorine, methyl, trifluoromethyl, hydroxy and methoxy,
and their salts, solvates and solvates of the salts.

4. Compound of the formula (I) according to claim 1, in which
either (i)
A represents C—R^{A}
and
D represents CH or N
or (ii)
A represents CH or N
and
D represents C—R^{D},
where R^{A} represents methyl, amino, methylamino or ethylamino,
and
R^{D} represents (C₁-C₄)-alkyl which is substituted by hydroxy or acetoxy or represents cyclopropyl which is substituted by hydroxy, hydroxymethyl or acetoxy or represents a group of the formula —NR¹R² or —C(═O)—NR³R⁴ where
R¹ and R² are attached to one another and together with the nitrogen atom to which they are attached form a 4- to 6-membered heterocycle which may contain a further heteroatom from the group consisting of N(R⁵) and O and which may be substituted by a radical selected from the group consisting of cyano, methyl and hydroxy, where
R⁵ represents methyl, ethyl, 2,2,2-trifluoroethyl, cyclopropyl or acetyl,
and
R³ and R⁴ independently of one another represent hydrogen or methyl
or
R³ and R⁴ are attached to one another and have the meanings of R¹ and R²,
Y represents CH or N,
Z represents CH
and
R^{p} represents trifluoromethoxy, trifluoromethylthio, 1,1,1-trifluoro-2-methylpropan-2-yl or 1-(trifluoromethyl)cyclopropan-1-yl,
and their salts, solvates and solvates of the salts.

5. Process for preparing a compound of the formula (I) as defined in claim 1, characterized in that a pyridinone- or pyridazinonecarboxylic acid of the formula (II) 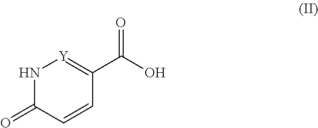
in which Y has the meaning given in claim 1,
is either [a]
alkylated in the presence of a base with a compound of the formula (III) 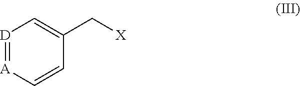
in which A and D have the meanings given in claim 1
and
X represents a customary leaving group such as, for example, chlorine, bromine, iodine, mesylate, triflate or tosylate
to give a compound of the formula (IV) 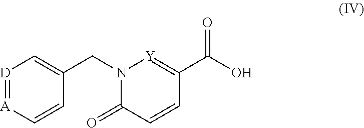
in which A, D and Y have the meanings given in claim 1,
and the carboxylic acid of the formula (IV) is then condensed with an N′-hydroxyamidine of the formula (V) 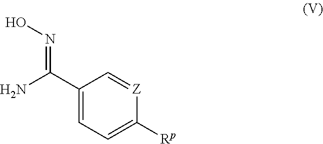
in which R^{p} and Z have the meanings given in claim 1
to give the 1,2,4-oxadiazole derivative of the formula (I) according to the invention 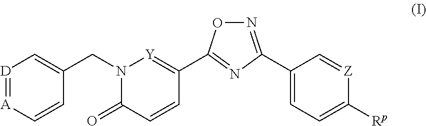
in which A, D, R^{p}, Y and Z have the meanings given in claim 1
or is [b]
initially reacted with an N′-hydroxyamidine of the formula (V) 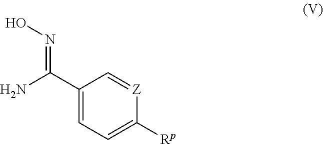
in which R^{p} and Z have the meanings given in claim 1
to give a 1,2,4-oxadiazole derivative of the formula (VI) 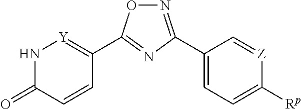
in which R^{p}, Y and Z have the meanings given in claim 1
and this is then alkylated in the presence of a base with a compound of the formula (III) 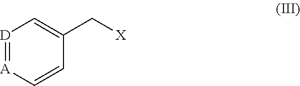
in which A and D have the meanings given in claim 1
and
X represents a customary leaving group such as, for example, chlorine, bromine, iodine, mesylate, triflate or tosylate
to give the compound of the formula (I) according to the invention 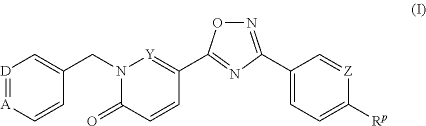
in which A, D, R^{p}, Y and Z have the meanings given in claim 1
and the resulting compound of the formula (I) is optionally separated into the enantiomers and/or diastereomers thereof and/or converted using appropriate (i) solvents and/or (ii) bases or acids to a solvate, salt and/or solvate of a salt thereof.

6. (canceled)

7. (canceled)

8. (canceled)

9. A method for the treatment of cancers or tumours comprising administering to a human or mammal in need thereof a therapeutically effective amount of a compound as defined in claim 1.

10. A method for the treatment and/or prevention of ischaemic cardiovascular diseases, heart failure, myocardial infarction, arrhythmia, stroke, pulmonary hypertension, fibrotic diseases of the kidney and lung, psoriasis, diabetic retinopathy, macular degeneration, rheumatic arthritis or Chuvash polycythaemia.

11. A pharmaceutical composition comprising a compound as defined in claim 1 in combination with one or more inert nontoxic pharmaceutically suitable excipients.

12. A pharmaceutical composition comprising a compound as defined in claim 1 in combination with one or more further active ingredients.

13. (canceled)

14. (canceled)

15. A method for the treatment of cancers or tumours comprising administering to a human or mammal in need thereof a therapeutically effective amount of a compound selected from the group consisting of:
1-(4-chlorobenzyl)-5-[3-(4-methylphenyl)-1,2,4-oxadiazol-5-yl]pyridin-2(1H)-one,
5-[3-(4-chlorophenyl)-1,2,4-oxadiazol-5-yl]-1-(4-methylbenzyl)pyridin-2(1H)-one,
5-[3-(4-methoxyphenyl)-1,2,4-oxadiazol-5-yl]-1-(4-methylbenzyl)pyridin-2(1H)-one
and
2-(4-methoxybenzyl)-6-[3-(4-methylphenyl)-1,2,4-oxadiazol-5-yl]pyridazin-3(2H)-one.

16. A method for the treatment and/or prevention of ischaemic cardiovascular diseases, heart failure, myocardial infarction, arrhythmia, stroke, pulmonary hypertension, fibrotic diseases of the kidney and lung, psoriasis, diabetic retinopathy, macular degeneration, rheumatic arthritis or Chuvash polycythaemia comprising administering to a human or mammal in need thereof a therapeutically effective amount of a compound selected from the group consisting of:
1-(4-chlorobenzyl)-5-[3-(4-methylphenyl)-1,2,4-oxadiazol-5-yl]pyridin-2(1H)-one,
5-[3-(4-chlorophenyl)-1,2,4-oxadiazol-5-yl]-1-(4-methylbenzyl)pyridin-2(1H)-one,
5-[3-(4-methoxyphenyl)-1,2,4-oxadiazol-5-yl]-1-(4-methylbenzyl)pyridin-2(1H)-one
and
2-(4-methoxybenzyl)-6-[3-(4-methylphenyl)-1,2,4-oxadiazol-5-yl]pyridazin-3(2H)-one.
